(19) United States
(12) Patent Application Publication
(10) Pub. No.: US 2007/0087959 A1
(43) Pub. Date: Apr. 19, 2007

(54) Method of inducing biomineralization method of inducing bone regeneration and methods related thereof

(75) Inventors: Charles Sfeir, Pittsburgh, PA (US); Phil Campbell, Pittsburgh, PA (US); Julie A. Jadlowiec, Pittsburgh, PA (US); Prashant Kumta, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: University of Pittsburgh of the Commonwealth System of Higher Education, Pittsburgh, PA (US); Carnegie Mellon University, Pittsburgh, PA (US)

(21) Appl. No.: 10/568,998

(22) PCT Filed: Aug. 19, 2004
(86) PCT No.: PCT/US04/27076

§ 371 Date: Dec. 18, 2006

Related U.S. Application Data
(60) Provisional application No. 60/496,245, filed on Aug. 19, 2003.

(51) Int. Cl. A61K 38/17; A61K 48/00; C07K 14/47

(52) U.S. Cl. 514/7; 514/44; 530/352

(57) ABSTRACT

A method of inducing biomineralization in a tissue, which method comprises administering to the tissue a source of Phosphophoryn (PP) in an amount sufficient to induce biomineralization; a method of treating tooth sensitivity or injured pulp tissue; a method of inducing differentiation of a cell into an osteogenic cell or odontogenic cell; a method of inducing bone or dentin regeneration; a method of inducing periodontal regeneration; a method of inducing differentiation of a cell into a cementoblast, osteoblast, or periodontal ligament cell; and a composition comprising a source of PP and a carrier.

### TECHNICAL FIELD OF THE INVENTION

The present invention pertains to a method of inducing biomineralization, a method of treating tooth sensitivity or injured pulp tissue, a method of inducing differentiation of a cell into an osteogenic cell or odontogenic cell, a method of inducing bone or dentin regeneration, a method of inducing periodontal regeneration, a method of inducing differentiation of a cell into a cementoblast, osteoblast, or a periodontal ligament cell, and a composition comprising a source of Phosphophoryn (PP) and a carrier.

### BACKGROUND OF THE INVENTION

Enamel, cementum and dentin are the three mineralized tissues of teeth. In human teeth, enamel covers the crown dentin, whereas cementum covers the root dentin. In turn, the dentin encloses the pulp of the tooth, which provides the dentin with vascular and neural support. Unlike enamel and cementum, the dentin is transversed by numerous tubules. The tubule walls are comprised of the calcified matrix of the dentin and the tubule space is filled with fluid (dentinal fluid) derived from pulp tissue fluid and serum. The matrix mineral is comprised mainly of the calcium phosphate salt, hydroxyapatite, which is poorly soluble at neutral and alkaline pH, and progressively more soluble as the pH becomes progressively more acidic.

Because of their rigid walls, the fluid that fills the narrow dentinal tubules enables cold, tactile, evaporative, and osmotic stimuli to be transmitted through the dentin to the pulp in the form of fluid movement. This movement of dentinal fluid is sensed as sharp pain of short duration. This pain is elicited when the odontoblasts that protrude into the pulpal ends of the tubules are disturbed and as a result, the mechano-receptors of the pulpal nerve fibers attached thereto are stimulated. The neural response is usually referred to as dentinal pain and the involved dentin as “hypersensitive” dentin.

Dentinal hypersensitivity, or tooth sensitivity, results when protective enamel or cementum covering dentin is lost. Cementum is easier to breach than enamel, because cementum is thinner and more easily eroded by acids. However, breach of cementum cannot happen until there is gingival recession and exposure of the root surface to the oral milieu. Individuals with breached cementum and suffering with dentinal hypersensitivity often experience pain when the exposed area of the tooth comes into contact with cold air, hot and cold liquids, foods that are sweet or acidic, or is touched with a metal object.

One way that loss of cementum occurs (and the same is true of enamel) is by the process of dental caries. Acids are produced as end-products of the bacterial degradation of fermentable carbohydrate and these acids dissolve hydroxyapatite, which, like dentin and enamel, is the main calcium phosphate mineral that comprises most of the mineral of the cementum. Another source is acidic foods, which, if ingested frequently and for prolonged periods of time, will cause tooth demineralization. These include fruit juices and many beverages, both alcoholic and non-alcoholic. Other acidic agents leading to chemical erosion include various oral personal care products. Amongst these are many of the commercially available mouthwashes and some toothpastes. Abrasive toothpastes and vigorous brushing can aid the erosion process. Another way in which dentinal tubules lose their protective cementum and enamel coverings is through procedures performed by the dentist or hygienist in the dental office. This includes cavity and crown preparation of teeth for fillings and other restorations. It also includes cementum removal during scaling and root planing by the periodontist or dental hygienist. (U.S. Pat. No. 6,482,395)

Many attempts have been made with limited success to treat tooth sensitivity (see, for example, U.S. Pat. Nos. 3,683,006; 5,139,768; 4,751,072; 4,631,185; and 6,524,558). Typically, such methods employ calcium salts and the like, and they are of limited efficacy. Accordingly, there is still a need for an improved method treating tooth sensitivity.

Bone and teeth are known to contain factors, which have the capacity to direct commitment of primordial mesenchymal cells towards cartilage and bone formation. Implantation of appropriately decalcified bone or dentin matrix into a soft tissue, such as a muscle pouch, induces bone formation through a process akin to endochondral ossification. Perivascular mesenchymal cells migrate to the implant and differentiate into cartilage, which then is replaced by true bone. (U.S. Pat. No. 4,935,497)

Chondrogenic/osteogenic-inducing factors, such as Bone Morphogenic Protein (BMP), have been used in methods of inducing differentiation of cells, e.g., stem cells, to cells of an osteogenic or odontogenic lineage that ultimately result in bone formation or bone regeneration in a tissue. However, superphysiological doses of BMP are needed to achieve a clinical response in vivo, which raises safety and manufacturing concerns. Thus, however, there still remains a need in the art for improved methods of inducing differentiation of a cell into an osteogenic cell or odontogenic cell, such that the method provides an improved method of inducing bone or dentin regeneration or bone formation.

Periodontitis occurs when inflammation or infection of the gums (gingivitis) is untreated or treatment is delayed. Infection and inflammation spreads from the gums (gingiva) to the ligaments and bone that support the teeth. Loss of support causes the teeth to become loose and eventually fall out. Periodontitis is the primary cause of tooth loss in adults. This disorder is uncommon in childhood but increases during adolescence.

Periodontitis affects the composition and integrity of periodontal structures at the dento-gingival junction, alveolar bone, cementum and periodontal ligament. It causes the destruction of connective tissue matrix and cells, loss of fibrous attachment and resorption of alveolar bone and often leads to tooth loss. The major goal of regeneration is to reverse the destructive effects of this disease. Successful periodontal regeneration has come to imply the formation of new cementum, new connective tissue attachment with functionally oriented periodontal ligament fibers, and coronal apposition of new supporting bone. Current concepts suggest that a population of progenitor cells within the periodontium constitute the cellular reservoir of new cells for repair and regeneration (Melcher et al., J Periodontol 47: 261-266 (1976)). This complex process involves migration, proliferation and selection of cells and their differentiation. It was also shown that cell-to-cell and cell-to-matrix interactions play an important role in the migration and local differentiation of the cells (Hynes et al., Cell 69: 11-25 (1992); and Hynes et al., Cell 68: 303-322 (1992)). However, the key factors, e.g., proteins, controlling these complex processes that lead to periodontal regeneration are largely unclear. Therefore, there exists a need in the art to determine the key factors involved in periodontal regeneration.

Guided tissue regeneration (GTR) has revolutionized the treatment of periodontal diseases, such as periodontitis. Numerous clinical and histological studies during the past decade have shown that periodontal tissue can be regenerated under specific conditions. GTR utilizes a resorbable or non-resorbable barrier to completely cover the periodontal defect, preventing the gingival epithelial and connective tissues from contacting the root surface. This barrier is placed in such a way that an empty space is created between the bony defect walls, the tooth surface and the barrier, allowing for clot formation, stabilization and tissue regeneration within that space. Although limited success has been achieved with GTR in treating periodontal diseases, there still exists a need in the art for improved methods of treating such diseases.

### BRIEF SUMMARY OF THE INVENTION

The present invention provides a method of inducing biomineralization in a tissue. The method comprises administering to the tissue a source of PP in an amount sufficient to induce biomineralization in the tissue.

The present invention also provides a method of treating tooth sensitivity or injured pulp tissue in a mammal. The method comprises administering to the mammal a source of PP in an amount sufficient to treat tooth sensitivity or injured pulp tissue.

Further provided by the present invention is a method of inducing differentiation of a cell into an osteogenic cell or odontogenic cell. The method comprises administering to the cell a source of PP in an amount sufficient to induce differentiation of the cell into an osteogenic cell or odontogenic cell.

A method of inducing bone regeneration in a tissue is also provided by the present invention. The method comprises administering to the tissue a source of PP in an amount sufficient to induce bone regeneration in the tissue.

Also, the present invention provides a method of inducing periodontal regeneration in a tissue. The method comprises administering to the tissue a source of PP in an amount sufficient to induce periodontal regeneration in the tissue.

Also, the present invention provides a method of treating periodontal diseases, such as peridontitis, in a patient. The method comprises administering to the tissue a source of PP in an amount sufficient to treat periodontal diseases in the patient.

A method of inducing differentiation of a cell into a cementoblast, osteoblast, or a periodontal ligament cell is further provided herein. The method comprises administering to the cell or a periodontal space a source of PP in an amount sufficient to induce differentiation of the cell into a cementoblast, osteoblast, or periodontal ligament cell.

The present invention further provides a composition comprising a source of PP and a carrier.

These and other objects and advantages of the invention, as well as additional inventive features, will be apparent from the description of the invention provided herein.

### BRIEF DESCRIPTION OF THE FIGURES

FIG. 1 represents the qPCR analysis of osteoblastic gene expression in hMSC, MC3T3.E1, and NIH3T3 cells. All three cell types were cultured in basal media or basal media containing recombinant human Bone Morphogenic Protein-2 (rhBMP-2) or recombinant Phosphophoryn (rPP). NIH3T3 cells were also genetically modified to produce transgenic PP. Total RNA was extracted after 2, 4 or 8 days in culture. Total RNA (10-30 ng) was subjected to qPCR analysis of Runx2, Osx, Bone/liver/kidney Alp, Ocn and Bsp genes. Results are expressed as fold over basal media control unless otherwise noted, FIG. 1A: Runx2 gene expression after 2 days: rPP increased Runx2 gene expression in hMSC (˜2.5-fold) and MC3T3.E1 cells (˜2.5-fold) and was equal to the level of rhBMP-2 for both cell types. Runx2 gene expression was not changed by rPP or transgenic PP (tPP) in NIH3T3. FIG. 1B: Osx gene expression after 4 days: Osx gene expression was not affected by rPP in MC3T3.E1 cells. rPP increased Osx gene expression (˜8-fold) in NIH3T3 cells only in the transgenic form. hMSC in basal media and rPP-treated did not express Osx. FIG. 1C: Total RNA signal of rhBMP-2-induced Osx. rhBMP-2-induced Osx gene expression in hMSCs over an 8 day time period. FIG. 1D: Alp gene expression after 8 days: rPP did not upregulate Alp gene expression for any of the cell types examined. FIG. 1E: Ocn gene expression after 8 days: rPP up-regulated Ocn gene expression in MC3T3.E1 (˜6-fold); tPP up-regulated Ocn gene expression in NIH3T3 to the level of rhBMP-2 (˜3-fold). Ocn was not up-regulated in hMSC by either rhBMP-2 or rPP alone. FIG. 1F: Bsp gene expression after 8 days. Neither hMSC nor NIH3T3 expressed Bsp for any treatment group. rhBMP-2 stimulated Bsp gene expression in MC3T3.E1. Neither rPP nor tPP up-regulated Bsp gene expression in any cell type examined after 8 days in culture. * Significantly higher than basal control, p<0.05; † Equal to rhBMP-2, p>0.05.

FIG. 2 represents the qPCR analysis of osteoblastic gene expression in hMSC cultured additionally with 100 nM Dex. FIG. 2A: Runx2 gene expression was increased (−10-fold) by Dex alone after 2 days in culture. Neither Dex+rhBMP-2 nor Dex+rPP increased Runx2 gene expression above Dex alone. FIG. 2B: hMSC expressed a low level of Osx after 4 days treatment with Dex. rhBMP-2 enhanced Osx gene expression (˜18-fold) over Dex alone. FIG. 2C: Dex alone did not increase Alp gene expression. Dex+rPP increased Alp gene expression slightly (˜2-fold) over basal control and Dex alone. FIG. 2D: Ocn gene expression was decreased by treatment with Dex for 8 days. rhBMP-2 and rPP did not further affect Ocn gene expression. FIG. 2E: hMSC treated with Dex for 8 days express detectable Bsp. There was no change in Bsp gene expression in Dex+rhBMP-2 or Dex+rPP vs. Dex alone. *Significant from basal control, p<0.05; † Significant from Dex alone, p>0.05.

FIG. 3 represents the qPCR analysis of Ocn in hMSC. Cells were cultured for 6 days in basal media or basal media containing rhBMP-2 or rPP in the absence of Dex. After 6 days, cells were then additionally supplemented with 10 nM Vitamin D₃ and cultured for an additional 48 hours. RNA was extracted and qPCR analysis was performed for Ocn. Cells treated with vitamin D₃ express increased levels of Ocn over basal media alone (12-fold). When rhBMP-2 was added, no significant change in Ocn gene expression was detected. rPP increased Ocn gene expression over basal media (˜36-fold) and vitamin D₃ alone (˜3-fold). *Significant from basal control, p<0.05; † Significant from vitamin D₃ alone, p>0.05.

FIG. 4 represents Ocn gene and protein analysis. Cells were cultured as before for 8 days. hMSC only were supplemented with 10 nM vitamin D₃ for the final 48 hours of culture (no serum added for OCN ELISA). A) qPCR analysis of Ocn gene expression. FIG. 4B: OCN protein release. Bars equal mean±SEM; n=3. *Significant from control, p<0.05

FIG. 5 represents Alkaline Phosphatase (ALP) activity after 14 days. hMSC only were cultured in media supplemented with 100 nM dex. ALP activity was calculated as U/mg total protein of the cell lysate. Bars equal mean±SEM; n=3. *Significant from control, p<0.05.

FIG. 6 represents an Alizarin red stain of hMSC. Cells were cultured as before for 28 days with 10 mM β-glycerophosphate and in the presence or absence of 100 nM dex.

FIG. 6A: Alizarin red stain for calcium. FIG. 6B: Quantification of alizarin red stain via extraction with 10% CPC in 10 mM phosphate buffer. Bars equal mean±SEM; n=3. *Significant from—dex control, † Significant from +dex control, p<0.05.

FIG. 7 represents (α_{ν}β₃ integrin blocking. hMSC were pre-treated with 10 μg/mL anti- α_{ν}β₃ for 1 hour and then supplemented with 50 μg/mL L-ascorbic acid phosphate with 100 ng/mL rhBMP-2 or 50 ng/mL rPP and cultured for 48 hours. qPCR analysis for Runx2 was performed. Gene expression is calculated as percent of unblocked control. Bars equal mean±SEM, n=3. *Significant from control, p<0.05

FIG. 8 depicts data that demonstrate activation of the MAP kinase pathway. hMSC and NIH3T3 were cultured with rPP for 10, 20, 30 and 60 minutes. Cell lysates were harvested and subjected to SDS-PAGE and probed for phosphor-p 38, phosphor-Erk1/2 and phosphor-Jnk by Western blotting.

FIG. 9 depicts staining for mineralization of NIH 3T3 cells. FIG. 9A: von Kossa stained control NIH 3T3 cells (Day 10) showing no mineralization. FIG. 9B: von Kossa stained transfected NIH 3T3 cells (Day 10) showing extensive mineralization.

FIG. 10 are von Kossa stainings. All three panels were stained at day 7 with von Kossa and counterstained with fuschin red. FIG. 10A: non-transfected cells (control). FIG. 10B: transfected cells showing multiple foci of mineral deposits. FIG. 10C: higher magnification of panel B (middle panel).

FIG. 11 is a simulated x-ray diffraction pattern for stoichiometric hydroxyapatite.

FIG. 12 is an X-ray diffraction pattern of the experimental sample isolated from the transfected cells.

FIG. 13 is a quantification of an alizarin red stain of NIH3T3 cells (white bars=control cells; black bars=PP-transfected). *significant from control, p<0.05; † significant from PP-transfected, AAP+Pᵢ, p<0.05.

FIG. 14 represents a graph of the mean alkaline phosphatase activity (U/mg total protein) for PP-transfected cells (black bars) and non-transfected cells (white bars), which have been administered either nothing (2 bars on the left) or AAP+Pi+rhBMP2 (2 bars on right), *significant from PP-transfected (control) and non-transfected (AAP+Pi+rhBMP-2), p<0.05,

FIG. 15 represents a graph of the mean fold over non-transfected cells of alkaline phosphatase gene expression in NIH3T3 cells for non-transfected (white bars) and PP-transfected (black bars) cells, which have been administered either nothing (2 bars on left) or AAP+Pi+rhBMP-2 (2 bars on right), *significant from control (basal), p<0.05; † significant from non-transfected (AAP+Pi+rhBMP-2), p<0.05.

FIG. 16 is a listing of all the sequences discussed herein.

FIG. 17 depicts X-ray crystallography data of NIH3T3 cells, which were not transfected with PP DNA. @ indicates NaCl.

FIG. 18 depicts X-ray crystallography data of MC3T3 cells, which were not transfected with PP DNA. No crystalline phase is shown.

FIG. 19 depicts X-ray crystallography data of NIH3T3 cells, which were transfected with PP DNA. * indicates HA; @ indicates NaCl; and b indicates brushite).

FIG. 20 depicts X-ray crystallography data of NIH3T3 cells, which were transfected with PP DNA. * indicates HA and @ indicates NaCl.

FIG. 21 depicts X-ray crystallography data of MC3T3 cells, which were transfected with PP DNA. * indicates HA and @ indicates NaCl.

FIG. 22 is a Western blot of phosphorylated Smad1. hMSC were cultured with rPP for 1, 20, 60, and 120 minutes. Cell lysates were harvested and subjected to SDS-PAGE and probed for phosphor-Smad1 by Western blotting.

FIG. 23 are fluorescence microscopy images of MDPC-23 and NIH3T3 cells transfected with ILK-GFP plasmid under the control of CMV promoter. Panel A: MDPC-23 control, where the cells were transfected with ILK-GFP but no rPP was added to the cell medium. The fluorescence is diffuse with only and few clusters of fluorescence. Panels B and C: NIH3T3 cells. Panels D and E are MDPC-23 cells that were all treated in similar fasion with rPP. Clustering can be seen, which is indicative of the formation of focal adhesion sites in response to rPP. These data indicate that ILK is activated once PP interacts with the integrin receptors.

### DETAILED DESCRIPTION OF THE INVENTION

PP

PP is a cleavage product of Dentin Matrix Protein 3 (DMP3), also known as Dentin Sialophosphoprotein (DSPP), comprising only Exon 5 of DMP3 and constitutes about 50% of the dentin extracellular matrix protein content. PP has also been localized to bone tissue, albeit in lesser quantities. The PP protein is highly anionic and its amino acid sequence consists mainly of aspartic acid-serine-serine (DSS) repeats. Approximately 85-90% of the serines are phosphorylated. Without being bound to any particular theory, the highly phosphorylated state of PP allows it to have high affinity for Ca²⁺ ions. PP also has an arginine-glycine-aspartic acid (RGD) sequence at the N-terminus. The coding sequence of the human PP gene and the amino acid sequence of the encoded gene product, i.e., the encoded protein, are publicly available at the National Center for Biotechnology Information (NCBI) website as GenBank Accession No. NM_{—}014208 (SEQ ID NO: 8) and NP_{—}055023 (SEQ ID NO: 7), respectively. The coding sequence of the Mus musculus PP gene and the amino acid sequence of the encoded gene product, i.e., the encoded protein, are set forth below as SEQ ID NO: 3 and 1, respectively.

Sources of PP: PP Proteins and PP Expression Vectors

Aspects of the present invention involve the administration of a “source” of PP in vivo or in vitro. The source of PP can be any source of PP, such as a PP protein, which can be provided to the desired target within a suitable formulation, or the source of PP can be an expression vector encoding a PP protein, which provides PP to the desired target by transfection, infection, or transduction of cells with the vector such that the cells produce the PP protein. For example, in one embodiment, the source of PP is a protein or peptide having, comprising, consisting essentially of, or consisting of the amino acid sequence of SEQ ID NO: 1, a fragment thereof, or a derivative of either of the foregoing. The fragment of the PP protein can be any suitable or desired fragment of SEQ ID NO: 1, preferably one that is functionally equivalent to the PP protein, i.e., retaining the same or similar function(s) of the PP protein, such as the ability to induce biomineralization, treat tooth sensitivity or injured pulp tissue, induce differentiation of a cell into an osteogenic cell or odontogenic cell, and/or induce bone or dentin mineralization, generation or regeneration as herein described. Preferably, the fragment of the PP protein has, comprises, consists essentially of, or consists of the amino acid sequence of SEQ ID NO: 2, which contains multiple DSS or aspartic acid-serine (DS) repeats. Without being bound by any particular theory, it is believed that the capacity of PP to induce mineralization (e.g., biomineralization) is attributed to the presence of such DSS repeats ([DSS]ₙ) in the protein and that the phosphorylation of the serines of the DSS repeats may also be of importance to the biomineralization. Thus, while not required for the practice of the inventive methods, it is preferred that a source of PP includes, or encodes a protein, comprising such DSS repeats. In this regard, the source of PP can alternatively be a DMP3 protein (or fragment thereof) of which Exon 5 is the PP protein, which contains multiple DSS repeats. Alternatively, the source of PP can be a derivative of the PP protein or fragment thereof. The term “derivative” as used herein refers to any functionally equivalent derivative of the PP protein or fragment thereof that has, comprises, consists essentially of, or consists of an amino acid sequence that is highly identical to that of the PP protein or fragment thereof and retains the same or similar function(s) of the PP protein or fragment thereof, i.e., the ability to induce biomineralization, treat tooth sensitivity or injured pulp tissue, induce differentiation of a cell into an osteogenic cell or odontogenic cell, and/or induce bone or dentin mineralization, generation or regeneration. Preferably, the amino acid sequence of the derivative is at least 75% identical to the amino acid sequence of the PP protein or the fragment thereof. More preferably, the amino acid sequence of the derivative is at least 85% identical to that of the PP protein or fragment thereof. Most preferably, the amino acid sequence is at least 95% identical to the amino acid sequence of the PP protein or fragment thereof.

The PP protein, fragment thereof, or derivative of either of the foregoing can be can comprise naturally occurring amino acids or non-naturally occurring amino acids, and can, furthermore, be modified, e.g., glycosylated, amidated, carboxylated, phosphorylated, esterified, N-acylated, or converted into an acid addition salt and/or optionally dimerized or polymerized. Moreover, the PP protein, fragment thereof, or derivative of either of the foregoing can be modified to create derivatives by forming covalent or non-covalent complexes with other moieties in accordance with methods known in the art. Covalently-bound complexes can be prepared by linking the chemical moieties to functional groups on the side chains of amino acids comprising the protein, fragment thereof, or derivative of either of the foregoing, or at the N- or C-terminus. Also, the protein, fragment thereof, or derivative of either of the foregoing can be made recombinantly or can be synthesized on a peptide synthesizer. Both methods of obtaining the protein, fragment thereof, or derivative of either of the foregoing are known in the art. See, for instance, Sambrook et al., 2001, supra; Modern Techniques of Peptide and Amino Acid Analysis, John Wiley & Sons (1981); and Bodansky, Principles of Peptide Synthesis, Springer Verlag (1984)).

Alternatively, the source of PP can be a nucleic acid molecule encoding a PP protein (SEQ ID NOs: 1 or 2), a fragment thereof, or a derivative of either of the foregoing. The term “nucleic acid molecule” as used herein is defined as a polymer of nucleic acids (e.g., DNA or RNA), (i.e., a polynucleotide), which can be single-stranded or double-stranded, synthesized or obtained from natural sources, and which can contain natural, non-natural or altered nucleotides and can contain natural, non-natural or altered internucleotide linkages. A variety of techniques used to synthesize the nucleic acid molecules of the present inventive methods are known in the art. See, for example, Sambrook et al., 1989, supra; and Lemaitre et al., Proc. Natl. Acad. Sci. USA 84: 648-652 (1987). Preferably, the nucleic acid molecule encoding the PP protein (SEQ ID NOs: 1 or 2), fragment thereof, or derivative of either of the foregoing has no insertions, deletions, inversions, and/or substitutions present. However, it may be suitable in some instances for the nucleic acid source of PP to comprise one or more insertions, deletions, and/or substitutions. For example, nucleic acid molecules having one or more insertions, deletion, and/or substitutions may have enhanced activity as compared to the nucleic acid molecule not having one or more insertions, deletions and/or substitutions. The nucleic acid molecule encoding the PP protein, fragment thereof, or derivative of either of the foregoing desirably has, comprises, consists essentially of, or consists of the nucleotide sequence of SEQ ID NO: 3 or SEQ ID NO: 4.

Where the source of PP is a nucleic acid molecule encoding a PP protein, it is desirable for such coding sequence to be operably linked to a native or non-native regulatory sequence. If more than one nucleotide sequence is included in the nucleic acid molecule, each sequence can be operably linked to its own regulatory sequence or to the same regulatory sequence (e.g., separated by internal IRES sites). The nucleic acid molecule (e.g., encoding PP) and the regulatory sequence are “operably linked” when they are functionally linked in such a way as to place the expression of the coding sequence under the influence or control of the regulatory sequence. Thus, a regulatory sequence is be operably linked to a nucleic acid molecule if the regulatory sequence effects transcription of that nucleic acid molecule and the resulting transcript is translated into the PP protein or polypeptide or other desired factor.

Within the expression construct, the “regulatory sequence” is typically a promoter sequence or promoter-enhancer combination, which facilitates the efficient transcription and translation of the nucleic acid to which it is operably linked (e.g., the source of PP or other factors). The regulatory sequence can, for example, be a mammalian or viral promoter, such as a constitutive or inducible promoter. Exemplary viral promoters, which function constitutively in eukaryotic cells include, for example, promoters from the simian virus, papilloma virus, adenovirus, human immunodeficiency virus, Rous sarcoma virus, cytomegalovirus, Moloney leukemia virus and other retroviruses, and Herpes simplex virus. Other constitutive promoters are known to those of ordinary skill in the art. The promoters useful as regulatory sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art and can be used in the context of the invention, when desired. The selection of promoters, e.g., strong, weak, inducible, tissue-specific and developmental-specific, is within the skill in the art. Similarly, the combining of a nucleic acid molecule as described above with a promoter is also within the skill in the art.

In addition to the regulatory sequence in operable linkage with the source of PP (or source of other desired factor(s)), the expression construct also can include other genetic elements. For example, a nucleic acid sequence encoding a signal peptide, such as GGATGGAGCTGTATCATCCTCTTCTTGGTAGCAACAGCTACA (SEQ ID NO: 11), which directs newly synthesized proteins to the endoplasmic reticulum (Kabat et al., Sequences of proteins of immunological Interest, Washington D.C.: U.S. Department of Health and Human Services, 1987) can be present in the construct. A nucleic acid sequence encoding a marker protein, such as green fluorescent protein or luciferase also can be present in the construct. Such marker proteins are useful in vector construction and determining vector transfection or transformation efficiency, as well as vector migration. Marker proteins also can be used to determine points of injection in order to efficiently space injections of a vector composition to provide a widespread area of treatment, if desired. Alternatively, a nucleic acid sequence encoding a selection factor, such as a negative election agent, which also is useful in vector construction protocols, can be part of the adenoviral vector. Where included. a preferred negative selection agent is an HSV tk gene cassette (Zjilstra et al., Nature, 342: 435 (1989); Mansour et al., Nature, 336: 348 (1988); Johnson et al., Science, 245: 1234 (1989): Adair et al., PNAS, 86: 4574 (1989); and Capecchi, M., Science, 244: 1288 (1989), incorporated herein by reference). Other negative selection genes will be apparent to those of ordinary skill in the art.

Where the source of PP (or other desired factor) is a genetic expression construct, it can be included within any suitable vector for use in transformation or transfection of any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)). Suitable vectors include plasmids, phagemids, cosmids, viruses, and other vehicles derived from viral or bacterial sources. Preferably, the vector is a plasmid or a viral vector. The viral vector can be any viral vector, such as an adenoviral vector, an adeno-associated viral vector, a retroviral vector, an SV40-type viral vector, a polyoma viral vector, an Epstein Barr viral vector, a papillomaviral vector, a herpes viral vector, a vaccinia viral vector, or a polio viral vector. Methods of constructing such vectors and employing them for expressing nucleic acids in vivo and in vitro are known in the art.

The source of PP can be associated with a substrate (incorporated into a substrate or deposited onto a substrate). The term “substrate” as used herein refers to any delivery system that allows sustained release, e.g., slow release, of the associated drug or agent. In one embodiment, the PP proteins or nucleic acid are incorporated into a matrix, such as a biomimetic extracellular matrix, which is a synthetic matrix that is intended to mimic a natural extracellular matrix in its structure and/or function. The matrix can be a polymer that is either a natural polymer or a synthetic polymer, or combinations thereof. The polymer can be a water-swellable polymer, optionally containing water within its matrix. The source of PP is mixed with the polymer precursors prior to or during polymerization of the polymer or its precursor, or prior to or during cross-linking of the polymers. In one embodiment, the source of PP is added to a solution containing a polymer and the polymer subsequently is cross-linked by standard methods. Including the PP in the cross-linking reaction and not in the polymer polymerization reaction ensures complete polymerization of the polymer, while incorporating the PP substantially homogenously in the matrix. The source of PP also can be loaded into a pre-formed-swellable polymer matrix. Examples of and methods for post-loading a compound into a hydrogels may be found in PCT Publication Nos. WO 01/91848 and WO 02/02182. Alternatively, the source of PP can be loaded onto/into a substrate by means of a printing device, e.g., an ink jet printer, or can be loaded by other computer-assisted manufacturing techniques (see, for instance, Cooley et al., Proceedings, SPIE Conference on Microfluidics and BioMEMS, pages 1-12 (October 2001)).

The source of PP can be a source of PP that is non-covalently associated with or within a natural or synthetic polymer matrix product. For example, the source of PP can be a formulation of a PP protein non-covalently linked to a fibrin matrix product or the source of PP can be an expression vector comprising a nucleic acid molecule encoding a PP protein or active fragments (SEQ ID NOs: 1 or 2) and a nucleic acid molecule encoding a fibrin matrix product, such that upon expression, the PP protein is non-covalently associated with the fibrin matrix product. Such expression vectors are particularly useful for administration to an osseous defect or a dentinal lesion. Additional natural or synthetic polymer matrices to which the source of PP can be non-covalently linked include, for instance, demineralized bone matrix preparations, injectable calcium phosphate cements, calcium sulfates, tricalcium phosphates, amorphous calcium phosphates, nanocrystalline and crystalline calcium phosphates, as well as calcium phosphate gels. Calcium phosphates can be synthesized in different crystallographic variations which exhibit different chemical and physical properties depending on the Ca/P ratio. Different modifications of calcium phosphates can be used for linking PP, which include brushite, monetite, hydroxyapatite, tricalcium phosphate, octacalcium phosphate, and carbonate substituted hydroxyapatite. Other substrates could include hyaluronic acid, gelatin, alginate, fibrin glue products, collagens, collagen-calcium-phosphate combinations, cyclodextrin, Poly-L-Lysine, and polymers (e.g., poly (lactide-co-glycolides), polylactides, polyglycolides, polyanhydrides, polyphosphazenes, polycarbonates, polyurethane, polycaprolactone, and other biodegradable polymers) and all such modifications. The carriers could be in the form of bulk solid, thin, film, fibers or as a gel surface. Other additional natural or synthetic polymer matrices are set forth below.

In addition to incorporating a source of PP into a natural or synthetic polymer, other sources of the substrates could include addition of crystalline, nanocrystalline or amorphous calcium phosphates (brushite, monetite, tricalcium phosphate, hydroxyapatite, octacalcium phosphate, carbonate substituted hydroxyapatite) into the natural or synthetic biodegradable polymeric formulations. PP could also be introduced into a calcium phosphate gel with varying Ca/P ratios.

One of ordinary skill in the art will readily appreciate that the source of PP can be modified in any number of ways, such that the therapeutic efficacy of the agent is increased through the modification. For instance, the source of PP could be conjugated either directly or indirectly through a linker to a targeting moiety. The practice of conjugating molecules, agents, or compounds to targeting moieties is known in the art. See, for instance, Wadwa et al., J Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the agent to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used herein, refers to any agent or molecule that bridges the source of PP to the targeting moiety. One of ordinary skill in the art recognizes that sites on the source of PP, which are not necessary for the function of the source, are ideal sites for attaching a linker and/or a targeting moiety, provided that the linker and/or targeting moiety, once attached to source of PP, does not interfere with the function of the source, i.e., the ability to induce biomineralization, induce differentiation of a cell into an osteogenic cell, induce bone/dentin regeneration, or treat tooth sensitivity or pulp capping procedure.

Method of Inducing Biomineralization

In one aspect, the present invention provides a method of inducing biomineralization in a tissue or cell culture. The method comprises administering to the tissue, or to cells in culture, a source of PP in an amount sufficient to induce biomineralization in the tissue or cell culture. As used herein, the term “administering” refers to both indirect and direct administration. In this regard, the phrases “administering to the cell” and “administering to the tissue” means that the administered agent (e.g., the source of PP) can be administered directly to the cell or tissue, or the agent can be administered to a juxtaposed, or even non-juxtaposed region, so long as the agent is eventually localized to the cell or the tissue, or is eventually localized to a space that is effective for achieving the desired result, e.g., differentiation of a cell into a cementoblast, osteoblast, or periodontal ligament cell, regeneration of bone, periodontal, or dentin tissue, induction of biomineralization, and treatment of tooth sensitivity.

The term “biomineralization” as used herein herein refers to the process of forming mineralized structures, such as may be found in the body of a living organism or in mineral deposits in cell culture. In biomineralization, typically crystals are produced by a heterogeneous nucleation mechanism. The deposition of mineral crystals in bone, dentin, cartilage and the like is orchestrated by cells and by mineral-matrix interactions. The affinity of extracellular matrix constituents for ions may control the formation of initial mineral deposits (nucleation) and may regulate the size, morphology, and orientation of resulting crystals (crystal growth). Mineralized structures include, for example, bone, teeth, and cartilage. The minerals comprising the structure can be any mineral, such as a calcium phosphate, e.g., hydroxyapatite, apatite, tri-calcium phosphate, calcium carbonate, brushite, monetite, octacalcium phosphate and the like. The extent to which biomineralization is induced can be assayed by alizarin red or Von Kossa staining or by other suitable technique.

By “inducing,” and words stemming therefrom, as used herein, is meant promoting or stimulating. The term “inducing” and the like does not necessarily imply a 100% or complete induction. Rather, there are varying degrees of induction of which one of ordinary skill in the art recognizes as having a potential benefit or therapeutic effect. Preferably, the present inventive methods cause an induction of biomineralization, differentiation, and/or bone regeneration to an extent that is at least 20% greater than the extent of induction achieved in the absence of administration of a source of PP. More preferably, the present inventive methods achieve induction to an extent that is at least 50% greater. Most preferably, the present inventive methods achieve induction to an extent that is at least 75% greater than the extent of induction achieved in the absence of administration of a source of PP.

While, as mentioned, the inventive method of inducing biomineralization can be used in vitro (e.g., to cells in culture), it also has therapeutic utility when employed in vivo. For example, induction of biomineralization in vivo in accordance with the present invention can assist in healing injured or fractured bone or dentin tissue or facilitate incorporation of a bone or dentin graft into a patient. Also, induction of biomineralization in vivo in accordance with the present invention can treat tooth sensitivity. For such applications, the source of PP is typically applied in the region of the junction between the graft and host bone or dentin structure, to the surface of a tissue, e.g., dentin, to the site of the fracture as appropriate, or to an osseous defect or dentinal lesion. Application or delivery of the source of PP need not be limited to such sites, however. In other embodiments, the inventive method can facilitate strengthening of bone or dentin within a patient, for example, by increasing mineralization within existing bone or dentin tissue. In this embodiment, the inventive method can enhance the strength of bone and/or dentin tissue, which can mitigate the effects of bone and/or dentin degenerative diseases or disorders (e.g., resulting from tooth decay, osteoporosis, or demineralization resulting from chemotherapy).

When employed in vitro, the method can result in mineralization in tissue culture cells or in cultured tissues (e.g., bone grafts, artificial matrices), cultured explanted organs, or cultured limb structures or primordia (including portions thereof, such as a limb bud, an arm or leg or portion thereof). In such embodiments, the inventive method can be employed in tissue regrowth and engineering. For example, the inventive method can increase mineralization in a bone or dentin graft (e.g., an explant or an artificial graft), which can be stored or later implanted into a patient. In other applications, the inventive method can be employed in an explanted limb primordia or structure to assist in growth, re-growth, or reconstruction of the limb in vitro, which then can be attached or re-attached to a patient.

Method of Inducing Differentiation

In another embodiment, the invention provides a method of inducing osteogenic or odontogenic differentiation of a cell. The method comprises administering to the cell a source of PP in an amount sufficient to induce differentiation of the cell into a cell of osteogenic lineage, such as an osteoblast or a preosteoblast, or of odontogenic lineage, such as an odontoblast or a pre-odontoblast.

The cell to be differentiated can be any cell type amenable to differentiation into an osteoblastic or odontoblastic lineage. Typically, the cell will be a fibroblastic cell type (e.g., a fibroblast cell line such as NIH3T3 cells) or a cell of mesenchymal lineage, such as pre-osteblast or pre-odontoblast. A most preferred cell type for differentiation in accordance with the inventive method include stem cells, such as mesenchymal stem cells and embryonic stem cells. The isolation of such cells (e.g., from bone marrow or the stromal fraction of other tissue types) is known in the art.

Differentiation of the cells in accordance with the inventive method is accomplished when the cells exhibit the expression of osteoblast-marker genes. In this sense, the invention can facilitate the expression of such genes, and the production of the gene products, within cells. Examples of such osteoblast-gene markers include bone sialoprotein, Runx2, bone/liver/kidney alkaline phosphatase, osteocalcin, (Latent Membrane Protein-1 (LMP-1), Latent Membrane Protein-3 (LMP-3) and Osterix. The expression of such genes in cells subject to treatment in accordance with the inventive method can be assessed by any standard method, e.g., northern hybridization, reverse transcription-polymerase chain reaction (RT-PCR), quantitative polymerase chain reaction (PCR) or measurements of the proteins (e.g., via Western blotting or immunohistochemistry). Alternatively, differentiation can be assessed by assessing the ability of the treated cells to deposit minerals, e.g., calcium, relative to untreated cells.

The method of inducing differentiation can be accomplished in vitro or in vivo. For in vitro use, the invention can facilitate the generation of a population of differentiated or partially differentiated osteoblasts or preosteoblasts. Such cells can be used in tissue engineering, for example by seeding grafts or tissue scaffolds for implantation into a patient. Indeed, such cells can be employed to grow, generate, or regenerate bone or dentin structures in vitro (e.g., bone or dentin grafts) that can be stored for later use or implanted into patients as needed. Alternatively, differentiated or partially differentiated osteoblasts or preosteoblasts generated in accordance with the inventive method can be implanted into patients, e.g., in the region of bony structures or teeth. In such embodiments, the differentiated or partially differentiated osteoblasts or preosteoblasts can facilitate bone or dentin healing, strengthening, generation, or re-generation in vivo.

Alternatively, the invention can be employed in vivo, to facilitate the development of differentiated or partially differentiated osteoblasts or preosteoblasts in vivo, which also can result in bone or dentin healing or generation within a patient. In accordance with this aspect, the source of PP is applied to cells in vivo in an amount and at a location sufficient to induce differentiation of cells in the location to an osteogenic lineage. In situ, the cells will facilitate the generation or regeneration of bone or dentin. Such in vivo methods can be used for the treatment of bone defects.

Method of Srengthening or Generating Bone or Dentin

Either through increasing mineralization or inducing osteogenic or odontogenic differentiation, the present invention provides a method of strengthening bone or dentin or inducing bone or dentin generation or regeneration. The method comprises administering to the tissue a source of PP in an amount sufficient to strengthen bone or dentin, or to induce bone or dentin generation or regeneration in the tissue. Alternatively, the method can involve transferring to the region of such tissue differentiated or partially differentiated osteoblasts or preosteoblasts created in accordance with the inventive method, as described above. Within the tissue, the osteoblasts or preosteoblasts differentiated in accordance with the inventive method will strengthen (e.g., by increasing mineralization), build, generate, or regenerate bone or dentin structures in vivo. Such a method can, for example, be employed to treat fractures within patients, or to treat conditions associated with weakened bone or dentin, such as osteoporosis or tooth sensitivity.

When used dentally, the inventive method can result in the generation, strengthening, and/or regeneration of dentin. An advantage of this method is that the invention can facilitate the strengthening and creation of dentin bridges in damaged teeth, which can retard and, in some patients, reverse the process of tooth decay. Indeed, in some patients, treatment with a source of PP can alleviate or repair dental damage so as to avoid traditional approaches such as root canal therapy. In addition, the inventive method, by inducing mineralization in teeth, can treat tooth sensitivity. The term “tooth sensitivity” as used herein refers to the condition in which the dentin structure is exposed by either the enamel of a tooth has worn away or is absent, or following periodontal disease and periodontal treatment that could lead to the loss or removal of the cementum layer exposing the dentin structure such that elements including cold temperature, hot temperature, extreme sweetness and the like, are sensed by the nerves of the tooth, resulting in a dental hypersensitivity of the animal to such elements. In this regard, the present invention also provides a method of treating tooth sensitivity or injured pulp tissue in a mammal. The method comprises administering to the mammal a source of PP in an amount sufficient to treat tooth sensitivity or injured pulp tissue. Desirably, the source of PP is administered to the teeth of such mammal, or to oral epithelial or dental pulp tissue within such mammal. The term “injured pulp tissue” as used herein refers to any pulp tissue that has been injured by any means, such as by a disease process, e.g., a carious lesion or genetic disease, or by a mechanical cutting or drilling process, e.g., such as those using rotary or hand instruments during dental care procedures.

Method of Strengthening or Generating Periodontal Tissue

The present invention provides a method of inducing differentiation of a cell into a cementoblast, osteoblast, or periodontal ligament cell. The method comprises administering to the cell or the periodontal space a source of PP in an amount sufficient to induce differentiation of the cell into a cementoblast, osteoblast, or periodontal ligament cell.

The cell to be differentiated can be any cell type amenable to differentiation into a cementoblastic, osteoblastic, or periodontal ligament lineage. The periodontal ligament (PDL) consists of two mineralized tissues, cementum and alveolar bone with an interposed fibrous, cellular and vascular soft connective tissue. PDL cell population is heterogeneous, consisting of two major lineages, fibroblastic and mineralizing tissues further divided into osteoblastic and cementoblastic subsets (Dubree et al., Abstract 1075 of the Pathogenesis Program of the IADR/AADR/CADR 82^{nd} General Session, Mar. 10-13, 2004). Typically, the cell to be differentiated will be a fibroblastic cell or a cell of a mesenchymal lineage. A preferred cell type for differentiation in accordance with the inventive method includes an undifferentiated cell, such as a stem cell (e.g., a mesenchymal stem cell, an embryonic stem cell, etc.). Such undifferentiated cells can be found, for example, in the periodontal ligament or bone marrow. Methods of isolating such undifferentiated cells are known in the art. Also, such cells are commercially available from the American Type Culture Collection (ATCC).

Differentiation of the cells in accordance with the present inventive method is accomplished when the cells exhibit expression of appropriate marker genes, which are expressed on cementoblasts, osteoblasts, or periodontal ligament cells. In this regard, the invention can provide a method of facilitating the expression of certain genes and the production of the proteins encoded by the genes, within the cells. Such marker genes are known in the art. For example, marker genes of cementoblasts include, for example, bone sialoprotein (BSP) and osteocalcin, whereas marker genes expressed by periodontal ligament cells include, for instance, Bone Morphogenic Protein-6 (BMP-6) and alkaline phosphatase (AP). Examples of marker genes for osteoblasts are any of the osteoblast-gene markers, as described herein. The expression of the genes can be assayed by any standard method, as described herein. Alternatively, differentiation can be determined by assessing the ability of the PP-treated cells to deposit minerals, e.g., calcium, relative to PP-untreated cells.

In the inventive method of inducing differentiation of a cell into a cementoblast, osteoblast, or periodontal ligament cell, the source of PP can be administered to a periodontal space, which is the region between the gum or bone and the tooth, or to the cell. Without being bound to any particular theory, the differentiation of the cell into a cementoblast, osteoblast, or periodontal ligament cell provides for the formation, repair, strengthening, or regeneration of periodontal tissue, which comprises cementum, bone, and periodontal ligament tissue. In this regard, the present invention also provides a method of inducing periodontal tissue formation, repair, strengthening, or regeneration in a tissue, as well as a method of inducing the formation, repair, strengthening, or regeneration of cementum, bone, and/or periodontal ligament tissue. The method comprises administering to the tissue a source of PP in an amount sufficient to induce periodontal tissue formation, repair, strengthening, or regeneration in the tissue.

The present inventive methods of inducing differentiation of a cell into a cementoblast, osteoblast, or periodontal ligament cell and of regenerating periodontal tissue can be carried out either in vivo, in vitro, or ex vivo. For example, the methods can comprise direct administration of a source of PP to a mammal. Alternatively, the methods can comprise contacting in vitro autologous cells from a mammal to be treated with a nucleic acid molecule encoding PP and subsequently transferring the treated cells back into the mammal. Such methods can be carried out to effectively treat a periodontal disease, such as periodontitis, which is a dental disorder that results from progression of gingivitis, involving inflammation and infection of the ligaments and bones that support the teeth. The methods also can be carried out to effectively facilitate or achieve guided tissue repair, which is a procedure that enables bone and tissue to re-grow around an endangered tooth or if the tooth is lost, to increase the amount of bone for implant placement.

Use of PP with Other Osteogenic Factors

While the source of PP can be employed alone to stimulate biomineralization, stimulate osteogenic differentiation, or to induce bone or dentin generation or regeneration, the source of PP also can be employed in connection with sources of other osteogenic factors. In this sense, the source of PP can act additively or, in some embodiments, even synergistically with one or more other osteogenic factors or growth factors to stimulate mineralization in cells and tissues, induce differentiation of cells towards osteogenic lineages and/or generate, regenerate, or strengthen bone and/or dentin structures in vitro or in vivo. Such other osteogenic factors or growth factors can be or comprise, for example: a BMP (e.g., such as the BMP having the amino acid sequence of SEQ ID NO: 9), a transforming growth factor (TGF), a latent TGF binding protein (LTBP), LMP-1, LMP-3, a heparin-binding neurotrophic factor (HBNF), growth and differentiation factor-5 (GDF-5), a parathyroid hormone (PTH), a fibroblast growth factor (FGF), an epidermal growth factor (EGF), a platelet-derived growth factor (PDGF), an insulin-like growth factor, a growth factor receptor, a cytokine, RunX, Osterix (Osx), a chemotactic factor, a granulocyte/macrophage colony stimulating factor (GMCSF), a LIM mineralization protein (LMP), a leukemia inhibitory factor (LIF), a hedgehog protein, an Insulin Growth Factor (IGF), a Vascular Endothelial Growth Factor (VEGF), and midkine (MK). Preferably, the other osteogenic factor is a BMP, a PDGF, an IGF, a VEGF, RunX, Osx, or an FGF. More preferably, the second osteogenic factor for use in the context of the present invention is a BMP, such as BMP-2, BMP-3, BMP-4, BMP-5, BMP-6, BMP-7, BMP-8, etc.

As with the source of PP, the “source” of such other osteogenic factor(s) can be the factors themselves (e.g., as protein preparations in a suitable composition) or genetic vectors encoding such factors. The source of PP and the source(s) of the other osteogenic factor(s) can be incorporated into the same formulation or matrix or administered simultaneously or sequentially, by the same route or a different route. With respect to the present inventive methods, the source of PP is preferably employed in combination with the source(s) of the other osteogenic factor(s). More preferably, the source of PP is employed in combination with a source of BMP.

Use of a source of PP in conjunction with a source of another osteogenic factor, particularly BMP, can circumvent many of the safety and manufacturing concerns pertaining to BMP. Less BMP may be required in such applications to elicit a response when PP also is employed, thus improving patient outcome.

Compositions for Administration of Sources of PP

With respect to the present inventive methods, the source of PP and/or source of other osteogenic factor(s) can be formed as a composition. In this regard, the present invention provides a composition comprising, consisting essentially of, or consisting of a source of PP and/or source of other osteogenic factor(s) and a carrier. Preferably, the composition comprises a PP protein having, comprising, consisting essentially of, or consisting of the amino acid sequence of SEQ ID NO: 1, a fragment thereof, or a derivative of either of the foregoing. More preferably, the composition comprises a fragment of a PP protein that has, comprises, consists essentially of, or consists of the amino acid sequence of SEQ ID NO: 2. Alternatively, the composition desirably comprises a nucleic acid molecule encoding a PP protein (e.g., encoding SEQ ID NOs: 1 or 2), a fragment thereof, or a derivative of either of the foregoing. The nucleic acid molecule can be in the form of an expression vector. More preferably, the nucleic acid molecule comprises, consists essentially of, or consists of the nucleotide sequence of SEQ ID NO: 3 or 4.

The composition can be or comprise any suitable form, depending on the nature of the source of PP (e.g., DNA or protein) and the type and location of cells to which the source of PP is applied. For example, where the source of PP is a nucleic acid, the composition can include lipid complexes, calcium phosphates gels, particles, or thin films, cyclodextrin, polyethyleneimines, etc., as are commonly used as transfection agents. Such transfectant agents also can be combined with a range of natural or synthetic polymers, such as discussed herein. Where the source of PP is a protein, the composition can include calcium phosphate gels, particles, or thin films, hydrogels, creams, etc., as are commonly employed for formulating and delivering proteins to cells. The invention includes such compositions and also methods of delivering a source of PP to cells or tissues using such compositions.

The composition of the present invention can be a pharmaceutical composition. Pharmaceutical compositions containing the source of PP and/or source of other osteogenic factor(s) can comprise more than one active ingredient, such as more than one source of PP, e.g., a PP protein and a fragment thereof and/or source of other osteogenic factor(s). The pharmaceutical composition can alternatively comprise a source of PP in combination with another pharmaceutically active agent or drug, e.g. another osteogenic factor, such as a source of BMP or other osteogenic factor(s). Preferably, the composition of the present invention comprises, consists essentially of, or consists of a source of PP and a source of another osteogenic factor. Desirably, the other osteogeneic factor is a BMP.

The composition comprising the source of PP preferably comprises a carrier. The carrier can be any suitable carrier. Preferably, the carrier is a pharmaceutically acceptable carrier. Also preferred is that the carrier is a biodegradable polymer, a biocompatible ceramic or a combination thereof. With respect to pharmaceutical compositions, the carrier can be any of those conventionally used and is limited only by chemico-physical considerations, such as solubility and lack of reactivity with the active compound(s), and by the route of administration. It will be appreciated by one of ordinary skill in the art that, in addition to the following described pharmaceutical compositions, the sources of PP and/or source of other osteogenic factor(s) of the present inventive methods can be formulated as inclusion complexes, such as cyclodextrin inclusion complexes, or liposomes. The carriers could also be biodegradable polymers and biocompatible ceramics or composites of both.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular source of PP and/or source of other osteogenic factor(s), as well as by the particular method used to administer the source of PP and/or source of other osteogenic factor(s). Accordingly, there are a variety of suitable formulations of the pharmaceutical composition of the present inventive methods. The following formulations for oral, aerosol, parenteral, subcutaneous, intravenous, intramuscular, interperitoneal, rectal, and vaginal administration are exemplary and are in no way limiting. One skilled in the art will appreciate that these routes of administering the agent or composition comprising the agent are known, and, although more than one route can be used to administer a particular agent, a particular route can provide a more immediate and more effective response than another route.

Injectable formulations can be used in accordance with the present invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630(1986)).

Topical formulations are well-known to those of skill in the art. Such formulations are particularly suitable in the context of the present invention for application to the skin or mucosa, particularly of the oral cavity. Formulations suitable for oral administration can consist of (a) liquid solutions, such as an effective amount of the source of PP dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. With respect to the present invention, the source of PP, or composition comprising the same, is preferably formulated into a toothpaste, a lozenge or hard candy, an oral rinse, a chewing gum, a dissolvable tablet or capsule, a dissolvable film, a gel, a natural biodegradable polymer, a synthetic biodegradable polymer, or a non-biodegradable polymer. The PP in a gel, cream, solution or other forms could be applied on the surface of the exposed dentin using a brush to reduce sensitivity.

Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphates containing varying Ca/P compositions, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The source of PP and/or source of other osteogenic factor(s), alone or in combination with another source which could be biocompatible calcium phosphate ceramics and gels of varying Ca/P ratios, biodegradable polymers and composites of calcium phosphate ceramics and gels and biodegradable polymers, and/or with other suitable components, can be made into aerosol formulations, which can be sprayed onto a region of bone (e.g., during an operation) or onto teeth or oral epithelial tissue. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer. The PP and carrier can also be cast into a thin film that can be placed into a region of bone or onto teeth or oral epithelial tissue.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The source of PP and/or source of other osteogenic factor(s) can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, a suspending agent, such as pectin, carbomers, methylcellulose, hydroxypropylmethylcellulose, carboxymethylcellulose, emulsifying agents and/or other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

Alternatively, the source of PP can be modified into a depot form, such that the manner in which the source is released into the body to which it is administered is controlled with respect to time and location within the body (see, for example, U.S. Pat. No. 4,450,150). Depot forms of sources of PP and/or source(s) of other osteogenic factor(s) can be, for example, an implantable composition comprising the source of PP and/or source of other osteogenic factor(s) and a porous material, such as a polymer or a ceramic gel or a polymer-ceramic composite gel, wherein the source is encapsulated by or diffused throughout the porous material. The depot is then implanted into the desired location within the body and the source of PP and/or source of other osteogenic factor(s) are released from the implant at a predetermined rate by diffusing through the porous material.

Polymer matrices of use as a tissue engineering substrate as described herein typically are “bioerodible” or “biodegradable” unless a permanent matrix is desirable. The terms “bioerodible” or “biodegradable” as used herein refer to materials, which are enzymatically or chemically degraded in vivo into simpler chemical species. Either natural or synthetic polymers can be used to form the matrix, although synthetic biodegradable polymers may be preferred for reproducibility and controlled release kinetics. U.S. Pat. Nos. 6,171,610, 6,309,635 and 6,348,069, which are incorporated herein by reference for their teachings regarding the art of tissue engineering, disclose a variety of matrices for use in tissue engineering. U.S. Pat. No. 6,171,610 discloses use of hydroxyapatite in tissue engineering. The hydroxyapatite prepared by the methods described herein is useful in such an application. In any case, the hydroxyapatite prepared by the methods described herein, for example complexed with a biomaterial such as plasmid DNA, may be associated with any suitable matrix, including without limitation those described herein. Biodegradable or bioerodible polymers can be used in conjunction with biocompatible calcium phosphate ceramics or gels. Different variants of calcium phosphate depending on the Ca/P ratio and the substitution of carbonate species for hydroxyl and the phosphate groups can be used either alone or in combination with the biodegradable polymers. Similarly, ceramic gels can be used either alone or in combination with the biodegradable polymers.

Natural polymers include, but are not limited to, fibrin, collagen, glycosaminoglycans (GAGs), such as chitin, chitosan and hyaluronic acid and polysaccharides, such as starch, ι-, κ- or λ-carrageenan, alginate, heparin, glycogen, agarose, and cellulose. In one embodiment, as shown for example below, a solution containing fibrinogen and nanocrystalline hydroxyapatite particles which are complexed with a transforming nucleic acid and are then cross-linked by the action of thrombin. Other natural polymers containing the nanocrystalline calcium phosphate particles of different Ca/P ratio, or complexes of nanocrystalline calcium phosphate particles with a biomaterial, are prepared in an equivalent manner, by mixing the hydroxyapatite or calcium phosphate complex with a polymer and then complexing the polymer with a cross-linker, or by any effective manner. The ceramic depending on the Ca/P ratio can thus be chemically equivalent to that of hydroxypatite, brushite, monetite, tricalcium phosphate (TCP), octacalcium phosphate (OCP), and carbonate substituted hydroxyapatite or calcium phosphate. In addition, partial substitution of the Calcium with divalent ions such as biocompatible Mg²⁺ can be introduced to induce stabilization of certain metastable phases such as TCP to facilitate better solubility or stability under physiological conditions. All of the above variants of the ceramic in the form of nanoparticles or nanocrystalline particles or amorphous nanosized particles or gels can be chemically complexed with the polymer or physically dispersed in the polymer to yield a casted sheet of fine particle suspension.

Synthetic polymers include, but are not limited to polylactide (PLA), polylactide-co-glycolide (PLGA), polycaprolactone (PCL), polyglycolic acid (PGA), polyurethanes, polycaprolactone, polymethyl methacrylate (PMMA), polyamino acids, such as poly-L-lysine, polyethyleneimine, poly-anhydrides, polypropylene-fumarate, polycarbonates, polyamides, polyanhydrides, polyortho esters, polyacetals, polycyanoacrylates and degradable polyurethanes. Useful non-erodible polymers include without limitation, polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, teflon.®, and nylon. Structurally, the polymers may have any useful form, including without limitation, hydrogels, dendrimers, polymeric micellular structures and combinations thereof. Synthetic polymers can be cross-linked or otherwise combined with natural polymers. The polymers can be cast into gels or as films. In addition, the polymers can be combined with the various ceramic compositions mentioned above.

For certain tissue engineering applications, attachment of the cells to the polymer is enhanced by coating the polymers with compounds such as basement membrane components, agar, agarose, gelatin, gum arabic, collagens types I, II, III, IV, and V, fibronectin, laminin, glycosaminoglycans, polyvinyl alcohol, mixtures thereof, and other hydrophilic and peptide attachment materials known to those skilled in the art of cell culture. It also may be desirable to create additional structure using devices provided for support, such as struts, or the like. These can be biodegradable or non-degradable polymers which are inserted to form a more defined shape than is obtained using the cell-matrices.

Another preferred formulation is a matrix material for tissue engineering, such as a demineralized bone graft or an artificial matrix comprising polymers or extracellular matric proteins. For example, a PP protein could be incorporated alone or in combination with other extracellular matrix proteins and/or other osteogenic proteins within such a matrix. Indeed, inclusion of PP into such matrices can circumvent many of the safety and manufacturing concerns pertaining to BMP. Less BMP may be required in such applications to elicit a response when PP also is incorporated into the matrix, thus improving patient outcome.

With respect to the present inventive methods, the mammal to be treated can be any mammal, including, but not limited to, mammals of the order Rodentia, such as mice, the order Logomorpha, such as rabbits, the order Carnivora, including Felines (cats) and Canines (dogs), the order Artiodactyla, including Bovines (cows) and Swines (pigs), the order Perssodactyla, including Equines (horses), the order Primates, Ceboids, or Simoids (monkeys) or of the order Anthropoids (humans and apes). An especially preferred mammal is the human. To some extent, the particular formulation and dosage of the source of PP and/or other osteogenic factor(s) and carriers including the biodegradable polymers and biocompatible ceramics will be informed by the species of mammal to be treated.

For purposes of therapeutic applications of the present inventive methods, the amount or dose of the source of PP and/or source of other osteogenic factor(s) and carriers administered should be sufficient to effect a therapeutic response in the animal over a reasonable time frame. The dose will be determined by the efficacy of the particular agent and the condition of the animal (e.g., human), as well as the body weight of the animal (e.g., human) to be treated. Many assays for determining an administered dose are known in the art. For purposes of the present inventive methods of inducing differentiation of a cell into an osteogenic cell or odontogenic cell and method of inducing bone or dentin regeneration, an assay, which comprises testing the induction of differentiation of a cell into an osteogenic cell upon administration of a given dose of a source of PP to a mammal among a set of mammals of which each is given a different dose of the source, can be used to determine a starting dose to be administered to a mammal. The extent to which differentiation is induced upon administration of a certain dose can be assayed by a gene reporter assay (see, for instance, the Examples set forth below). For purposes of the present inventive method of inducing biomineralization and method of treating tooth sensitivity, an assay, which comprises testing the induction of biomineralization upon administration of a given dose of a source of PP to a mammal among a set of mammals of which each is given a different dose of the source, can be used to determine a starting dose to be administered to a mammal. The extent to which biomineralization is induced upon administration of a certain dose can be assayed by a alizarin red staining or Von Kossa staining of cells expressing PP as described in the Examples set forth below.

The dose also will be determined by the existence, nature and extent of any adverse side effects that might accompany the administration of a particular source of PP. Ultimately, the attending physician will decide the dosage of the source of PP with which to treat each individual patient, taking into consideration a variety of factors, such as age, body weight, general health, diet, sex, inhibitor to be administered, route of administration, and the severity of the condition being treated.

### EXAMPLES

The following further illustrates the invention but, of course, should not be construed as in any way limiting its scope.

Abbreviations

For convenience, the following abbreviations are used herein: PP, Phosphophoryn; NCBI, National Center for Biotechnology Information; ECM, extracellular matrix; BMP, Bone Morphogenic Protein; DMP, Dentin Matrix Protein 3; DSS, aspartic acid-serine-serine; DS, aspartic acid-serine; RGD, arginine-glycine-aspartic acid; ATCC, American Type Tissue Collection; HLB, hydrophile-lipophile balance, PCR, polymerase chain reaction; RT-PCR, reverse transcription-polymerase chain reaction; LMP-1, Latent Membrane Protein-1; LMP-3, Latent Membrane Protein-3; TGF, Transforming Growth Factor; HBNF, heparin binding neutrophic factor; LTBP, Latent TGF Binding Protein; GDF-5, Growth and Differentiation Factor-5; PTH, Parathyroid Hormone; FGF, Fibroblast Growth Factor; EGF, Epidermal Growth Factor; PDGF, Platelet Derived Growth Factor; GMCSF, Granulocyte/Macrophage Colony Stimulating Factor; LMP, Osx, Osterix; LIM Mineralization Protein; LIF, Leukemia Inhibitory Factor; MK, Midkine; IGF, Insulin Growth Factor; VEGF, Vascular Endothelial Growth Factor; GAG, glycoasaminoglycan; PLA, polylactide; PLGA polylactice-co-glycolide; PGA, polyglycolic acid; PMMA, polycaprolactone, polymethyl methacrylate; qPCR, quantitative realtime polymerase chain reaction; Amp, ampicillin; IPTG, isopropyl β-D-thiogalactopyrasnoside; GST, glutathione S-transferase; tPP, transgenic Phosphophoryn; rhBMP-2, recombinant human Bone Morphogenic Protein-2; hMSC, human adult mesenchymal stem cells; rPP, recombinant PP; ELISA, enzyme-linked immunosorbent assay; SEM; standard error mean; MAP; Mitogenic Activated Protein; SDS-PAGE, sodium dodecyl sulfate-polyacrylamide gel electrophoresis; AAP, alkaline phosphatase; Pi, inorganic phosphate; RGD, arginine-glycine-aspartate; HA, hydroxyapatite; FTIR, Fourier transform infrared; EDAX, energy dispersive X-ray.

Materials

The following materials were used herein.

Vectors for production of recombinant PP (pGEX) and stable transfection (pShooter-ER) were obtained from Amersham (Piscataway, N.J.) and Invitrogen (Carlsbad, Calif.) respectively. BL21 cells were obtained from Invitrogen. LB, ampicillin and IPTG were obtained from Sigma (St. Louis, Mo.). Thrombin, Glutathione Sepharose 4B and ρ-aminobenzamindine Sepharose 4 Fast Flow was obtained from Amersham. FuGene Transfection Reagent was obtained from Roche (Palo Alto, Calif.). Human adult mesenchymal stem cells (hMSC) were obtained from BioWhittaker, Inc. (Walkersville, Md.). Human mesenchymal stem cell medium (MSCM), mesenchymal cell growth supplement, L-glutamine, penicillin and streptomycin were obtained from BioWhittaker, Inc. and added to the medium according to the manufacturer's specifications to prepare complete MSCM. MC3T3-E1 (clone 4) and NIH3T3 cells were obtained from ATCC (Manassas, Va.). DMEM, FBS, penicillin/streptomycin and trypsin-EDTA were obtained from GibCo BRL (Carlsbad, Calif.). 1, 25-(OH)₂ vitamin D₃ was obtained from Biomol (Plymouth Meeting, Mass.). Antibody to α_{ν}β₃ integrin (anti-α_{ν}β₃) was obtained from Chemicon (Temecula, Calif.). OCN ELISA kit was obtained from Zymed Laboratories (San Fransisco, Calif.). PBS, ALP assay kits, alizarin red-S and cetylpyridinium chloride (CPC) were obtained from Sigma Diagnostics, Inc. Total protein assay kits were obtained from Bio-Rad (Hercules, Calif.). RNeasy Kit and DNase I were obtained from Qiagen (Valencia, Calif.). RiboGreen Kit was obtained from Molecular Probes (Eugene, Oreg.). All quantitative real time PCR reagents, primers and probes were purchased from Applied Biosystems (Foster City, Calif.). Protease inhibitors were purchased from Pierce Biotechnology (Rockford, Ill.). Anti-phospho-p 38, anti-phospho-Erk1/2 and anti-phospho-Jnk were purchased from Cell Signaling Inc. (Beverly, Mass.). Western Lightning Chemiluminescence reagents were purchased from Perkin-Elmer (Boston, Mass.).

Cell Culture

The following describes how cells used herein were cultured.

hMSC, MC3T3-E1 and NIH3T3 were plated in 35 mm culture wells and grown in basal media to ˜70% confluence. Basal media for stem cells was complete MSCM and for MC3T3-E1 and NIH3T3, DMEM supplemented with 10% FBS and 1% penicillin/streptomycin. Cells were then treated with either rhBMP-2 (MC3T3-E1 and NIH3T3: 50 ng/mL; hMSC: 100 ng/mL) or 50 ng/mL rPP in basal media. The 50 ng/mL rPP concentration was determined based on a pilot experiment using a dose curve of 50, 100 and 250 ng/mL of rPP for MC3T3 and NIH3T3 cells. Cells were cultured for 2, 4 and 8 days prior to RNA extraction. Media was renewed every second day. Where noted, hMSC were also cultured in the presence of 100 nM dexamethasone (dex) as the negative control. We chose these time points to analyze the gene expression based on recommendations from the literature (Frank et al., J Cell Biochem 85, 737-746 (2002); Jaiswal et al., J Cell Biochem 64, 295-312 (1997)).

Statistical Analyses

For qPCR assays, the coefficient of variation (COV) was calculated from three assay replicates. For all treatment groups and target genes analyzed, the COV did not exceed 3%. All experiments were performed at least twice and one representative experiment is reported as the mean of three treatment triplicates±standard error of the mean (SEM). One-way analysis of variance (ANOVA) followed by Fisher's LSD multiple comparison post hoc test using SYSTAT 9 software (Richmond, Calif.) was performed to determine significance among treatment groups. A p-value <0.05 was considered statistically significant.

### Example 1

This example demonstrates the generation of recombinant PP and a transfected cell line.

Isolated mouse genomic PP was used as a template to amplify by PCR exon 5. The primers used were designed with Sal I and Xba I at the 5′ ends of the gene specific sequence (bold letters). Five random bases 5′ to the restriction site were inserted to allow Sal I and Xba I digestions. The primers used were: Forward: 5′ CTAATGTCGACATGGAGAGTGGCAGCCGTGGAGA 3′ (SEQ ID NO: 12); Reverse: 5′ GCATTCTAGATTAAAGCACCCGCCATTCAAATCG 3′ (SEQ ID NO: 13). The thermocycling conditions were as follows: Three cycles of 94° C. for 70 sec (denaturation), 52° C. for 70 sec (annealing), 72° C. for 2 minutes (extension) followed by 30 cycles of 94° C. for 70 sec (denaturation), 62° C. for 70 sec (annealing), 72° C. for 2 minutes (extension). The obtained PCR fragment was inserted into the pGEX-4T-3 vector and transformed into the bacterial host BL21. Cells were cultured in LB+Amp media for 4 hr at 30° C. Protein expression was induced by 1 mM IPTG for 3 hr. The bacterial lysate was cleared by centrifugation and applied directly to Glutathione Sepharose 4B. After washing with PBS, GST-bound protein was eluted with thrombin. Thrombin was removed from eluates with ρ-aminobenzamindine immobilized on Sepharose 4 Fast Flow matrix. The purified protein was electrophoresed on a 12% polyacrylamide gel and stained with Stains All to verify the molecular mass. A strong band was visible at ˜55 Kda that corresponds to the correct mass of PP. The amino acid composition of the purified protein was determined and was found to match the cloned sequence. Recombinant PP (rPP) was stored at −80° C. until use. PP PCR product was also inserted into the pShooter-ER vector and transfected into NIH3T3 using FuGene 6 Transfection Reagent. Stably transfected cells were selected using G418. Expression of the PP gene was verified by RT-PCR and PP protein secretion by dot blot analysis. The anti-PP was a generous gift from Dr. Arthur Veis at Northwestern University (Veis et al., Microsc Res Tech 59, 342-351 (2002)). PP produced by stably-transfected NIH3T3 is denoted tPP.

This example demonstrated that a recombinant PP protein was made. This example further demonstrated that cells stably transfected with PP DNA expressed and secreted the PP protein.

### Example 2

This example demonstrates that Phosphophoryn up-regulates osteoblast marker genes.

Total RNA was extracted using the RNeasy Kit with DNase I treatment according to the manufacturer's protocol. RNA content was determined using the RiboGreen RNA Quantification Kit. Conventional RNA quantification using 260/280 absorbance readings proved to be too imprecise to match the specificity of quantitative real-time PCR. Total RNA content was photometrically analyzed with a Tecan Spectrafluor platereader (Research Triangle Park, N.C.) with excitation at 485 nm and emission at 595 nm. RNA concentrations were calculated based on a standard curve of control ribosomal RNA.

Cells were harvested from the culture treatments at the time points described above. After extraction and quantification of RNA, quantitative realtime PCR (qPCR) analysis was carried out using Taqman® One-step RT-PCR Master Mix. Total RNA (10-30 ng) was added per 50 μL reaction with sequence specific primers (50-200 nM) and Taqman® probes (100 nM). Sequences for all target gene primers and probes are shown in Table 1. 18S primers and probes were designed by and purchased from Applied Biosystems. qPCR assays were carried out in triplicate on an ABI Prism 7000 Sequence Detection System. Thermocycling conditions were as follows: 48° C. for thirty minutes (reverse transcription), 95° C. for 10 minutes (initial denaturation) followed by 40 cycles at 95° C. for 15 seconds (denaturation) and 60° C. for 45 seconds (annealing and extension). The threshold was set above the non-template control background and within the linear phase of target gene amplification to calculate the cycle number at which the transcript was detected (denoted C_{T}).

TABLE 1

| | | | | |
|---|---|---|---|---|
| | Accession | | | |
| Gene | Number | Forward primer | Reverse primer | Taqman ® probe |

| | | | | |
|---|---|---|---|---|
| Human Runx2 | NM 004348 | AACCCACGAATGCACTATCCA | CGGACATACCGAGGGACATG | CCTTTACTTACACCCCGCCAGTCACCTC |
| Human Osx | AF 477981 | CCCCACCTCTTGCAACCA | CCTTCTAGCTGCCCACTATTTCC | CCAGCATGTCTTGCCCCAAGATGTCTA |
| Human Alp | XM 001826 | CCGTGGCAACTCTATCTTTGG | GCCATACAGGATGGCAGTGA | CATGCTGAGTGACACAGACAAGAAGCCC |
| Human Ocn | NM 000711 | AGCAAAGGTGCAGCCTTTGT | GCGCCTGGGTCTCTTCACT | CCTCGCTGCCCTCCTGCTTGG |
| Human Bsp | NM 004967 | AACGAAGAAAGCGAAGCAGAA | TCTGCCTCTGTGCTGTTGGT | AAAACGAACAAGGCATAAACGGCACCA |
| Mouse Runx2 | NM 009820 | AAATGCCTCCGCTGTTATGAA | GCTCCGGCCCACAAATCT | AACCAAGTAGCCAGGTTCAACGATCT |
| Mouse Osx | NM_130458 | CCCTTCTCAAGCACCAATGG | AGGGTGGGTAGTCATTTGCATAG | CAGGCAGTCCTCCGGCCCC |
| Mouse Alp | XM 124424 | CCGATGGCACACCTGCTT | GAGGCATACGCCATCACATG | CGGCGTCCATGAGCAGAACTACATTCC |
| Mouse Ocn | NM 007541 | CCGGGAGCAGTGTGAGCTTA | AGGCGGTCTTCAAGCCATACT | CCCTGCTTGTGACGAGCTATCAG |
| Mouse Bsp | L 20232 | ACCCCAAGCACAGACTTTTGA | CTTTCTGCATCTCCAGCCTTCT | TTAGCGGCACTCCAACTGCCC |

Gene expression values were calculated based on the comparative δδC_{T} method (separate tubes) detailed in Applied Biosystems User Bulletin #2 (Lee et al., Biochem Biophys Res Commun 309, 689-694 (2003). For each primers/probe set, validation experiments demonstrated that efficiencies of target and reference gene amplification were approximately equal; the absolute value of the slope of log input amount vs. C_{T}<0.1. Target genes were normalized to the reference housekeeping gene, 18S. Fold differences were calculated for each treatment group using normalized C_{T} values for the negative control at the appropriate time point as the calibrator. If no baseline expression of target gene was detectable, then total amount of RNA was calculated.

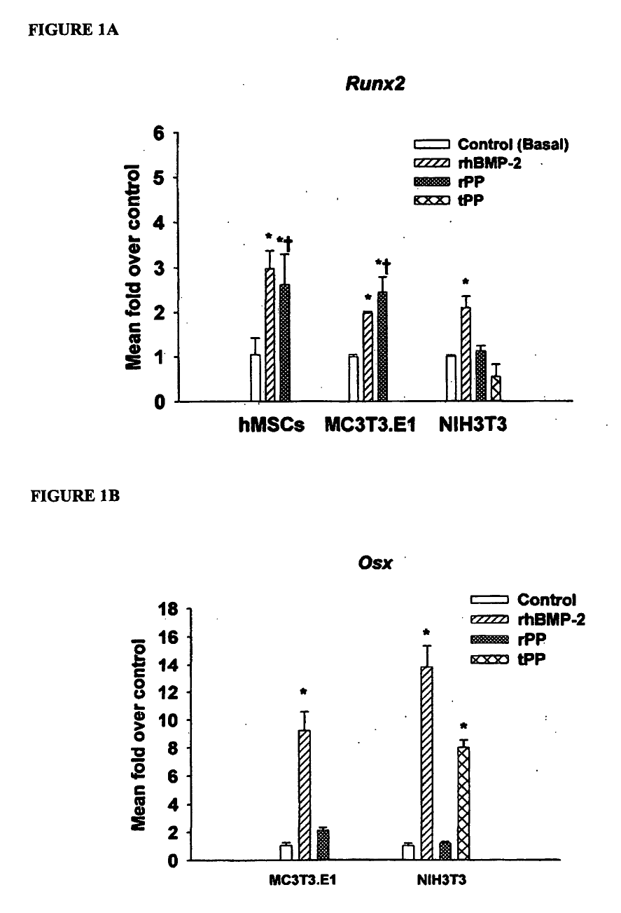

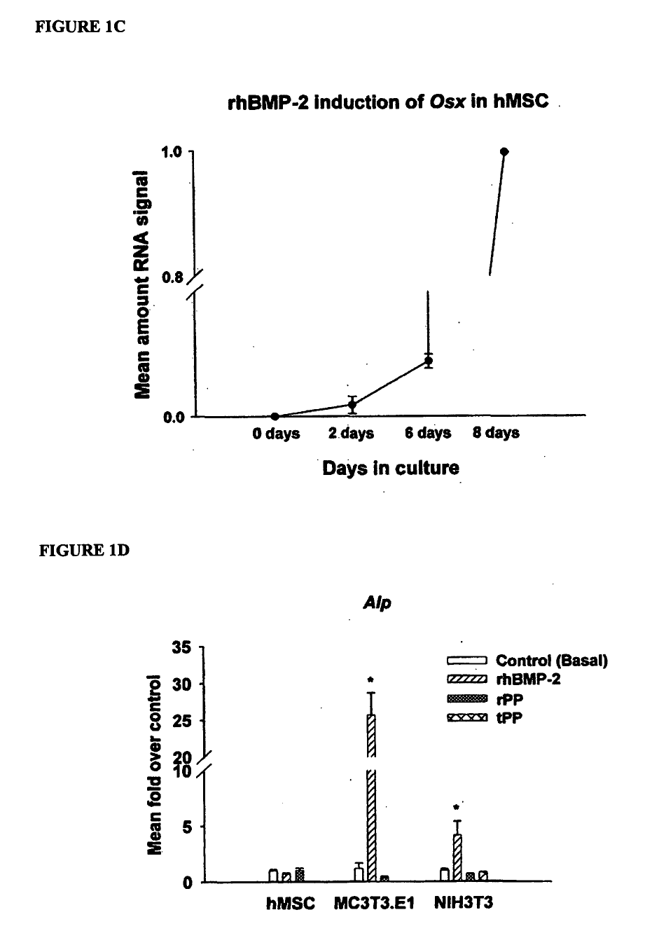

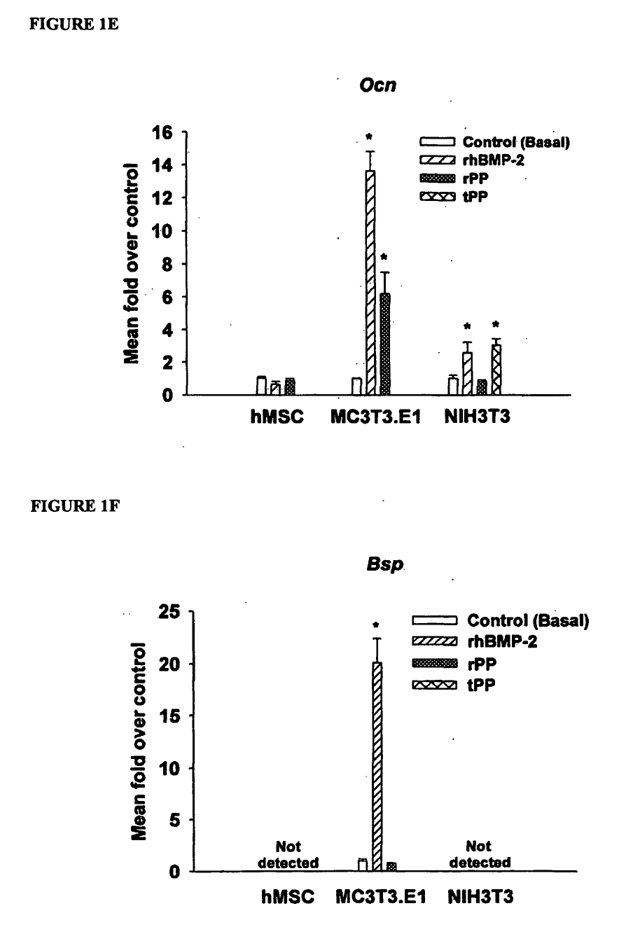

To determine the role of PP in osteoblast gene expression, qPCR was used to measure expression of Runx2, Osx, Alp, Ocn and Bsp in hMSC, MC3T3.E1 and NIH3T3 cells in response to rPP. NIH3T3 cells were also genetically modified to produce transgenic PP (tPP). Our positive control was cells treated with rhBMP-2 and our negative control was basal medium. Runx2 gene expression qPCR analysis was performed on RNA harvested after 2 days in culture. Runx2 gene expression was increased over basal medium control in both hMSC (˜2.5-fold) and MC3T3.E1 (˜2.5-fold) (FIG. 1a). Runx2 gene expression was not enhanced in NIH3T3 cells by either rPP or tPP. Further, in hMSC and MC3T3.E1, Runx2 gene expression induced by rPP was equal to that induced by rhBMP-2.

Osx gene expression analysis was performed on RNA extracted after 4 days in culture. rhBMP-2 increased Osx gene expression ˜9-fold in MC3T3.E1; whereas rPP did not affect Osx gene expression in this cell line (FIG. 1b). Although Osx was up-regulated in NIH3T3 by tPP ˜8-fold over basal media control, it did not exceed the level of induction by rhBMP-2 (˜13-fold). rPP did not affect Osx gene expression in NIH3T3. Both MC3T3.E1 and NIH3T3 express basal levels of Osx that permits a “fold over control” calculation for these two cell lines. However, hMSC do not express a basal expression of the Osx message. By qPCR analysis, Osx gene expression was below the threshold of detection. Therefore, induction of Osx gene expression by rhBMP-2 is shown as a graph of total amount RNA signal vs. days in culture (FIG. 1c). Over a period of 8 days in media containing rhBMP-2, Osx gene expression steadily increases. Upon treatment with rPP, Osx gene expression was not detectable for a period of up to 8 days in culture.

Alp, Ocn and Bsp gene expression analysis was performed on RNA from all cell lines extracted after 8 days in culture. Alp gene expression was up-regulated in both MC3T3.E1 and NIH3T3 by rhBMP-2 (˜25-fold and ˜4-fold respectively) but not by rPP or tPP (FIG. 1d). Although hMSC express a basal level of Alp message, no change was detectable for either rhBMP-2 or rPP in hMSC over a period of 8 days. Ocn gene expression was enhanced by rPP in both MC3T3.E1 (˜6-fold) and NIH3T3 (˜3-fold) (FIG. 1e). Up-regulation of Ocn by tPP in NIH3T3 was equal to that of rhBMP-2. hMSC express a low level of Ocn message, however, no change was detected with treatment of either rhBMP-2 or rPP alone over a period of 8 days. Bsp gene expression was not affected by rPP in MC3T3.E1; whereas, our positive control, rhBMP-2 increased Bsp gene expression −20-fold over basal media control (FIG. 1f). NIH3T3 and hMSC cells did not express Bsp message for any treatment group over an 8 day culture period. hMSC are traditionally cultured in medium containing Dex to guide the cells toward osteoblastic lineage (Wang et al., Proc Natl Acad Sci USA 95, 14821-14826 (1998)). Dex was not initially included in the cultures to avoid possible synergistic action between Dex and rhBMP-2 or rPP that may mask any slight changes in gene expression (Lukashev et al., Trends Cell Biol 8, 437-441 (1998); Adams et al., Development 117, 1183-1198 (1993)). However, Runx2 was the only gene for which we detected a change in expression stimulated by either rhBMP-2 or rPP in the absence of Dex in hMSC. Therefore, qPCR analysis of the above marker genes was performed on another set of cultures (hMSC only) containing 100 nM Dex.

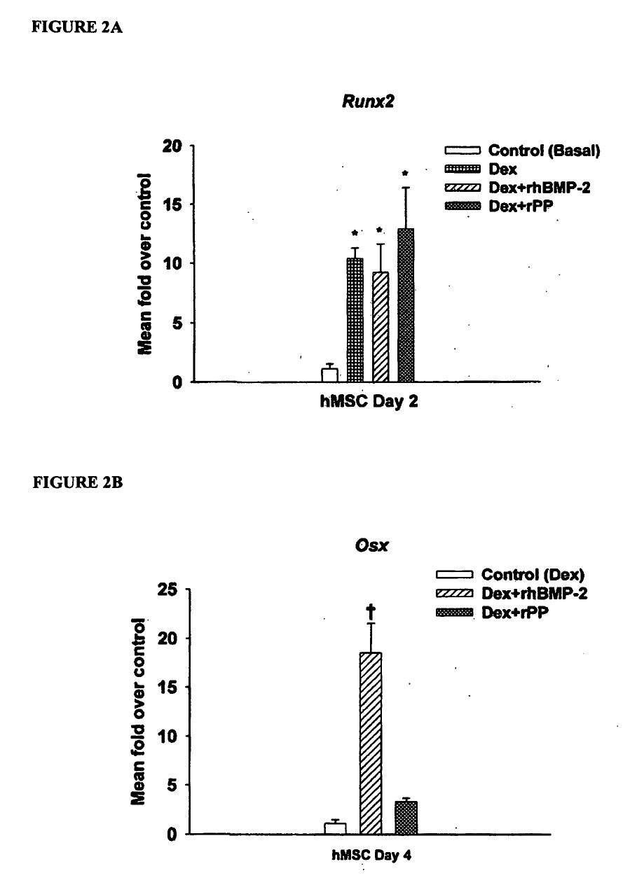

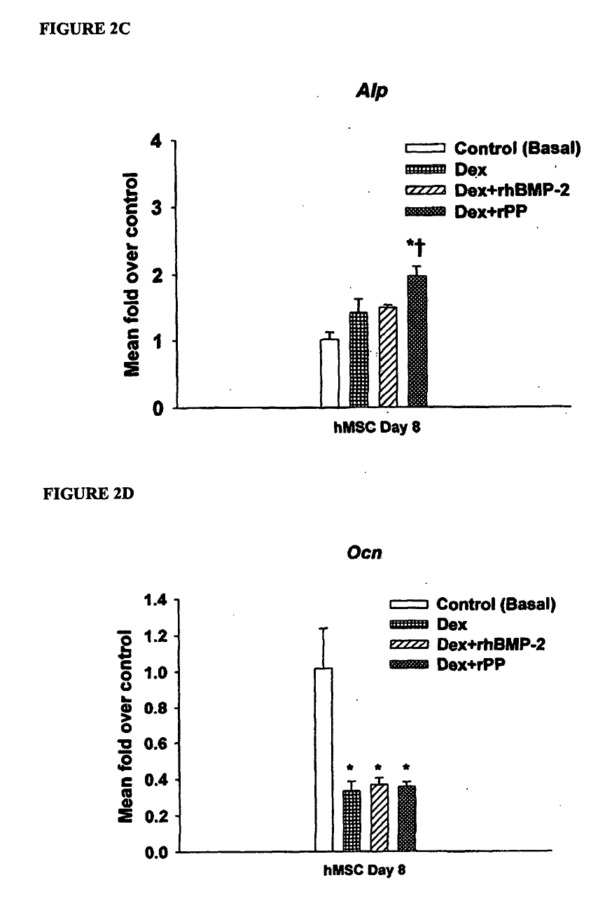

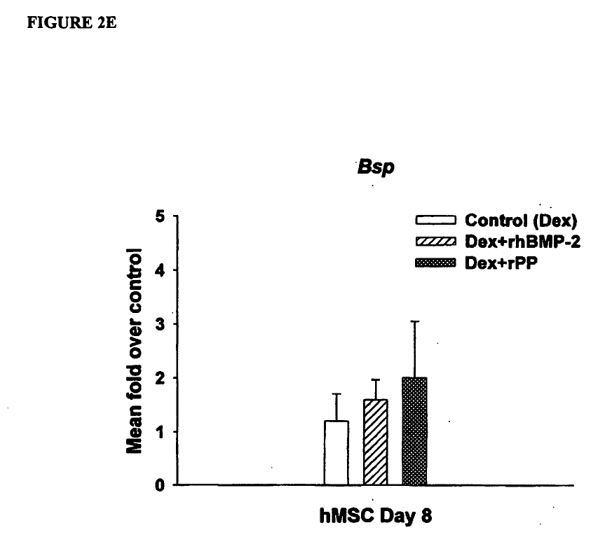

Addition of Dex to the basal media significantly increased Runx2 expression ˜10-fold compared to basal media alone after 2 days in culture (FIG. 2a). However, treatment of rhBMP-2 and rPP with Dex did not further enhance Runx2 gene expression over Dex alone. It is possible that the cells have reached their peak Runx2 expression due to treatment with Dex and further stimulation by rhBMP-2 or rPP does not result in higher levels of Runx2 message but rather may feedback to inhibit further Runx2 gene expression. Osx showed a different profile than Runx2. Whereas, hMSC do not express basal levels of Osx, treatment with Dex for 4 days resulted in extremely low, but nonetheless detectable Osx message. Additional treatment with rhBMP-2 further increased Osx gene expression in hMSC ˜18-fold over Dex alone (FIG. 2b). However, rPP had no additional effect on Osx expression over Dex alone-treated hMSC. After 8 days in culture, Alp gene expression was not enhanced by treatment with Dex, nor with additional supplementation with rhBMP-2 (FIG. 2c). However, the combination of Dex and rPP in the media resulted in slightly elevated Alp gene expression (˜2-fold) over both basal media control and Dex alone. Ocn gene expression was adversely affected by treatment with Dex after 8 days in culture demonstrating ˜60% reduction in Ocn message compared to basal media control. Previous studies by others have shown that in hMSC, osteogenic media containing Dex reduced expression of both Ocn message and protein induced by Vitamin D₃ (Wang et al. (1998), supra). In another report however, rat bone marrow-derived stem cells demonstrated increased Ocn message in response to a combination of Dex and rhBMP-2 and required vitamin Da (Adams et al. (1993), supra). No detectable change in Ocn gene expression by additional treatment with either rhBMP-2 or rPP was found. Following treatment with Dex for 8 days, Bsp gene expression was low, but detectable as was the case with Osx. Additional treatment with either rhBMP-2 or rPP did not enhance further Bsp expression. Bsp is usually expressed at later stages of differentiation, peaking just before matrix mineralization (Byers et al., J Bone Miner Res 17, 1931-1944 (2002)). No change in Bsp expression was observed even after 21 days in culture, in the absence and presence of Dex.

As reported by others, vitamin D₃ is essential for expression of both Ocn message and protein in hMSC (Wang et al. (1998), supra; Adams et al. (1993), supra). However, other reports have shown induction of Ocn gene expression by Dex and FGF-2 in human bone marrow-derived stem cells (Byers et al. (2002), supra) and OCN protein in rat bone marrow-derived stem cells by rhBMP-2 alone without inclusion of vitamin D₃ in the culture media (Lukashev et al., (1998), supra). As shown herein, Dex alone and in combination with either rhBMP-2 or rPP actually decreased Ocn gene expression in hMSC. In the absence of Dex, there is no difference in Ocn gene expression among basal media, rhBMP-2- and rPP-supplemented media. Despite conflicting reports on the use of Dex and vitamin D₃ alone, in combination or with other growth factors, vitamin D₃ was included in the culture media in the absence of Dex to determine the effect of rPP on Ocn gene expression in hMSC.

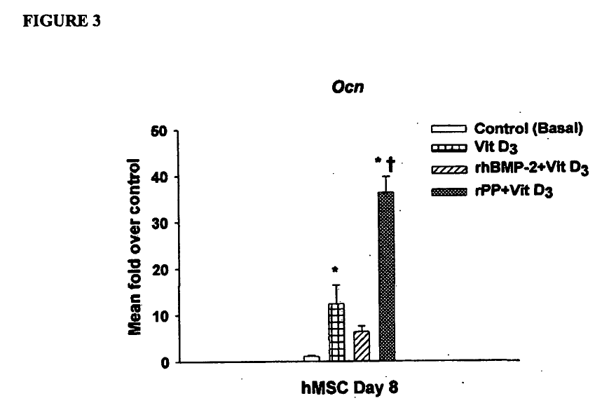

When vitamin D₃ was added to the basal media of hMSCs, Ocn gene expression increased ˜12-fold over cells cultured in basal media alone after 8 days in culture (FIG. 3). When cells were additionally supplemented with rhBMP-2, no change in Ocn expression was detected, as has been reported by others (Ducy et al., Cell 89, 747-754 (1997). However, when treated with rPP in the presence of vitamin D₃, hMSC expressed higher levels of Ocn gene expression than either basal media (˜36-fold) or Vitamin D₃ alone (˜3-fold).

This example demonstrated that PP plays a role in progression of mesenchymal stem cells and osteoprecursors towards a more mature cell.

### Example 3

This example demonstrates PP induced Ocn gene expression and protein production.

hMSC were cultured as before for 8 days in basal media or basal media plus 100 ng/mL rhBMP-2 or 50 ng/mL rPP and supplemented with 10 nM 1,25-(OH)₂ vitamin D₃ for the final 48 hours of culture (Jaiswal et al., J Cell Biochem 64, 295-312 (1997). MC3T3-E1 and NIH3T3 were cultured similarly but did not require vitamin D₃ for induction of Ocn. Total RNA was extracted as described above and analyzed via qPCR for Ocn gene expression. For OCN ELISA, cells were cultured in rhBMP-2 or rPP-containing medium for 8 days. For the final 48 hours of culture, cells were cultured in media without serum added. Conditioned media was collected and stored at −80 C until use. OCN ELISA was performed according to the manufacturer's instructions. OCN concentration (ng/mL) was calculated from a standard curve and normalized to total protein of the cell lysate as determined by the Bio-Rad Protein assay.

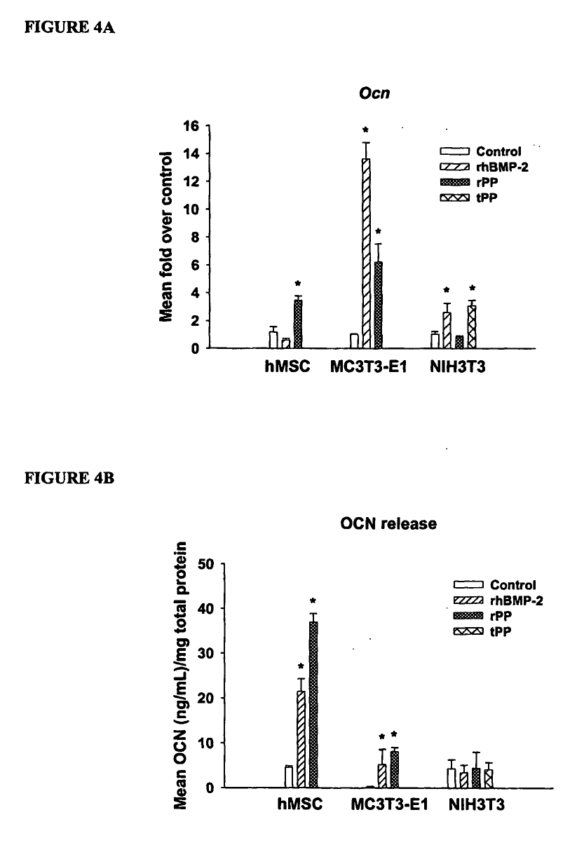

PP up-regulated Ocn gene expression over control in all three cell types (p<0.05) (hMSC: 4-fold, MC3T3-E1: 6-fold and NIH3T3: 3-fold) (FIG. 4A). PP increased OCN protein release ˜10-fold above negative controls (p<0.05) for both hMSC (40 versus 4 ng/mL) and MC3T3-E1 (10 versus 1 ng/mL) (FIG. 4B). There was no change in OCN protein release in NIH3T3 due to PP treatment.

This example demonstrated that PP increased the activity of ALP, a common marker of osteogenic lineage progression.

### Example 4

This example demonstrates Phosphophoryn increased ALP activity in hMSC.

Cells were cultured in the above media, supplemented with 10 mM β-glycerophosphate for all treatment groups. hMSC were additionally supplemented with 100 nM dex. Cells were harvested by trypsinization and centrifugation after 7, 14, and 28 days in culture. MC3T3-E1 and NIH3T3 were cultured similarly without dex. Cell pellets were resuspended in 500 μL of lysis buffer. Cell lysates were frozen at −80° C. for at least 2 hours prior to ALP activity assays. Five microliters of thawed cell lysates were incubated with 200 μL ALP 10 reagent from the Sigma Diagnostics Kit for 30 minutes at 37° C. An initial absorbance reading (time 0) was taken at 405 nm prior to thirty minute incubation at 37° C. and following (time 30). ALP activity was calculated according the manufacturer's instructions. ALP activity was normalized to total protein of the cell lysate.

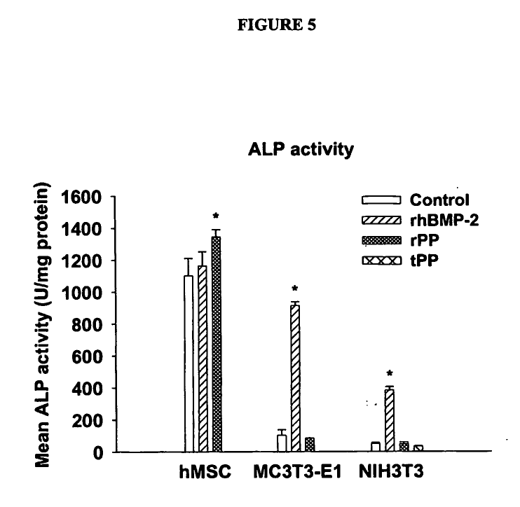

To further examine the function of PP in osteogenic lineage progression, ALP activity, which is a common phenotypic marker for osteogenesis, was examined. ALP activity was measured over a time course of 7 to 28 days. FIG. 5 shows the highest increase in ALP activity in hMSC at day 14. In contrast, rPP-treated groups did not increase ALP activity in MC3T3-E1 and NIH3T3, as well as the stably-transfected NIH3T3 over the time course examined. The positive control, rhBMP-2, increased ALP activity over basal media control for all three cell types.

### Example 5

This example demonstrates Phosphophoryn increased calcium deposition in hMSC.

hMSC were cultured as before in 100 nM dex and 10 mM β-glycerophosphate-containing media for 28 days. Cells were fixed in 70% ice-cold ethanol for 1 hour and rinsed with ddH₂0. Cells were stained with 40 mM alizarin red-S, pH 4.2 for 10 minutes with gentle agitation. Cells were rinsed five times with ddH₂0 and then rinsed for 15 minutes with 1×PBS and gentle agitation. Alizarin red was extracted from fixed cells by treatment with 500 μL 10% CPC for 20 minutes with gentle agitation. Absorbance of extracted alizarin red in CPC solution was measured at 570 nm. Amount of alizarin red (in μg) was determined according to an alizarin red standard curve and normalized to total protein of the cell lysate.

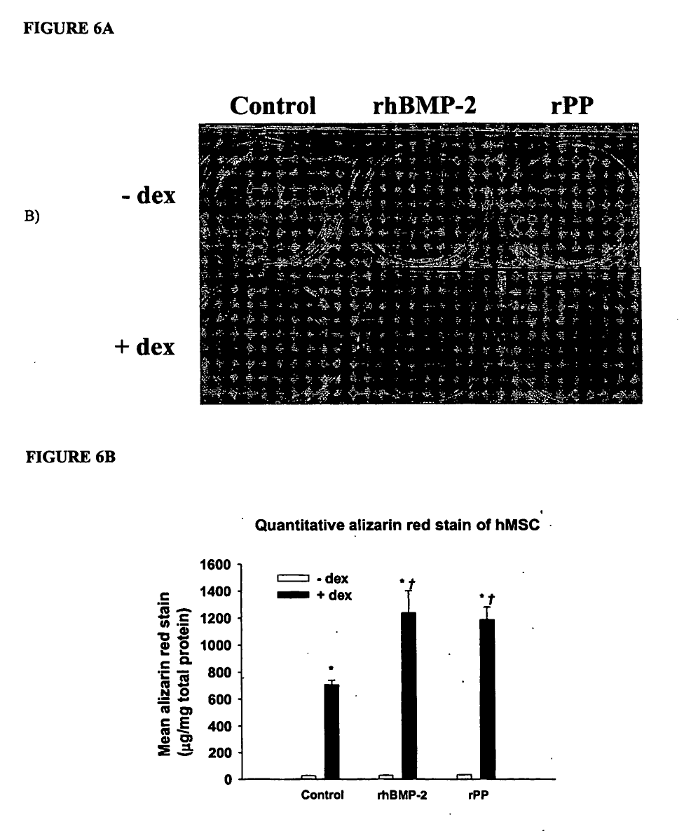

In hMSC, when added in combination with 100 nM dex and 10 mM β-glycerophosphate, both rhBMP-2 and rPP demonstrated increased alizarin red staining after 28 days in culture (FIG. 6A). Without dex, no alizarin red is detected. Upon quantification of alizarin red stain with 10% CPC, dex alone, dex+rhBMP-2 and dex+rPP exhibited increased alizarin red staining over cultures that did not contain dex (FIG. 6B). Further, rhBMP-2 and rPP demonstrated increased alizarin red staining in cultures supplemented with dex, compared to hMSC cultured with dex alone. This data reinforces the above-posed question as to the role of PP in signaling and/or mineral deposition. Therefore, the signaling mechanism of PP via integrin/RGD interactions was investigated.

This example demonstrated that treatment of cells with PP caused an increase in calcium deposition.

### Example 6

This example demonstrates that PP regulates gene expression via the α_{ν}β₃ integrin.

hMSC were seeded into 35 mm culture wells and allowed to reach ˜70% confluence in basal media. Cells were washed twice with 1X sterile PBS and treated with 15 or 25 μg/mL anti-α_{ν}β₃ diluted in serum-free basal media in a total volume of 500 μL. Cells were incubated with rocking for one hour at 37° C. Cells were then washed twice with 1X sterile PBS and incubated in media containing 50 μg/mL L-ascorbic acid phosphate and either 100 ng/mL rhBMP-2 or 50 ng/mL rPP for 48 hours. Total RNA was extracted and Runx2 gene expression analyzed via qPCR.

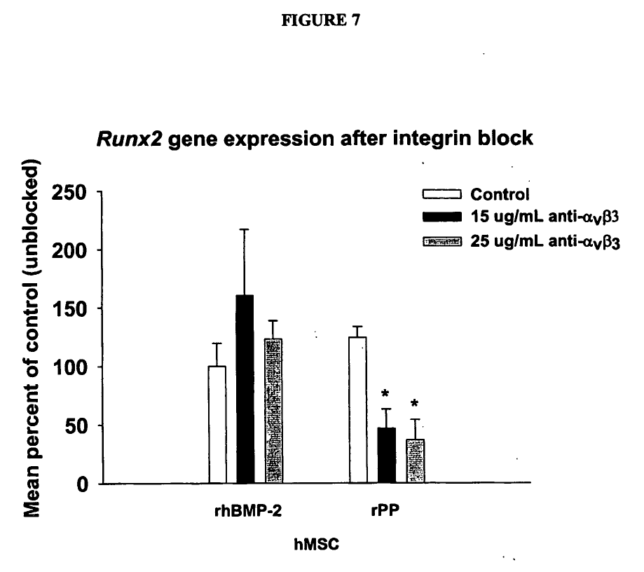

PP has an RGD domain; therefore, it was hypothesized that PP may be functioning in osteoblastic gene expression via binding to the α_{ν}β₃ integrin on the cell surface. As demonstrated above, rPP stimulated Runx2 gene expression in hMSC. Therefore, it was decided to test the integrin interaction hypothesis on hMSC using a blocking antibody to the α_{ν}β₃ integrin. Upon addition of the integrin blocking antibody, Runx2 gene expression due to rPP was decreased by ˜60% compared to un-inhibited control (FIG. 7). Runx2 gene expression was not inhibited by anti-α₈₄ β₃ in the rhBMP-2-treated hMSC as BMP-2 acts via it own specific receptors (Type I and II).

This example demonstrated that PP functions to up-regulate bone specific gene markers via binding to the α_{ν}β₃ integrin and triggering intracellular signaling pathways.

### Example 7

This example demonstrates that PP regulates gene expression via the MAPK pathways.

hMSC and NIH3T3 were cultured in triplicates as specified above except that the cells were cultured overnight in serum-free media prior to rPP treatment. Conditions for treatment (time) were based on recommendations from the literature (Higuchi et al., Bone Miner Res 17, 1785-1794 (2002); Lee et al., Oncogene 21, 7156-7163 (2002); Xiao et al., J Biol Chem 277, 36181-36187 (2002); and Lai et al., J Biol Chem 276, 14443-14450 (2001)). rPP (250 ng/ml) was added for 10, 20, 30 and 60 minutes and the cells were lysed on ice in RIPA buffer (150 mM NaCl, 1% Igepal CA-630, 0.5% sodium deoxycholate, 0.1% SDS, 50 mM Tris, pH 8.0) in the presence of protease inhibitors. The lysed cells were then pooled and protein concentration was determined. The lysates were stored at −80° C. at least 2 hours prior to use. Total protein (30 μg) were loaded onto 10% SDS gels and subjected to SDS-PAGE. Gels were blotted onto PVDF membranes and probed with anti-phosphorylated p38 (1:250), anti-phosphorylated Erk1/2 (1:250) and anti-phosphorylated Jnk (1:250) by Western blotting. Bands were detected by chemiluminescence of HRP and exposure to X-OMAT Kodak film was performed.

It should be noted that 50 ng/mL rPP was the optimal dose for gene expression experiments, although treatment of cells with 250 ng/mL rPP yielded the optimum induction of MAPK components, in comparison to the lower concentration of 50 and 100 ng/mL of rPP. For the gene expression experiments, 50 ng/mL rPP was probably sufficient due to the time of rPP-treatment and the high sensitivity of qPCR assay. Whereas for the western blotting technique, a shorter treatment time seemed to require a higher dose.

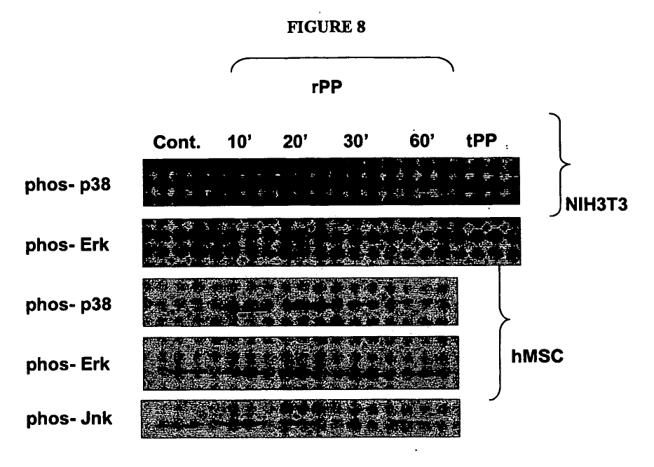

To further study the signaling pathway following PP interaction with the α_{ν}β₃ integrin receptor, the involvement of the MAPK pathway and more specifically the activation of p38, ERK1/2 and Jnk was investigated. As shown in FIG. 8, the p38 activation was apparent within 10 minutes of exposure to rPP in both NIH3T3 and hMSC cells. The ERK1/2 pathway seems to be only active in NIH3T3 cells whereas the hMSC cells were not activated compared to the control cells. When Jnk was assessed for its activation, it was evident that there was a positive response at 10 minutes. These data clearly show that rPP is signaling via the MAPK pathway demonstrated by the phosphorylation of p38, ERK1/2 and Jnk. The gene activation data shown in this paper by quantitative PCR is likely due to the activation of the MAPK pathway and the translocation of its components to the nucleus where they activate transcription of target genes.

This example demonstrated that PP signals through the MAPK pathway, thereby potentiating growth factor activity.

### Example 8

This example demonstrates that expression of PP induce mineral deposition.

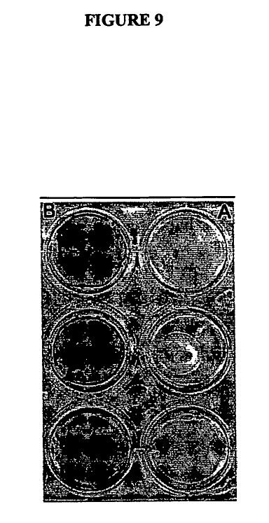

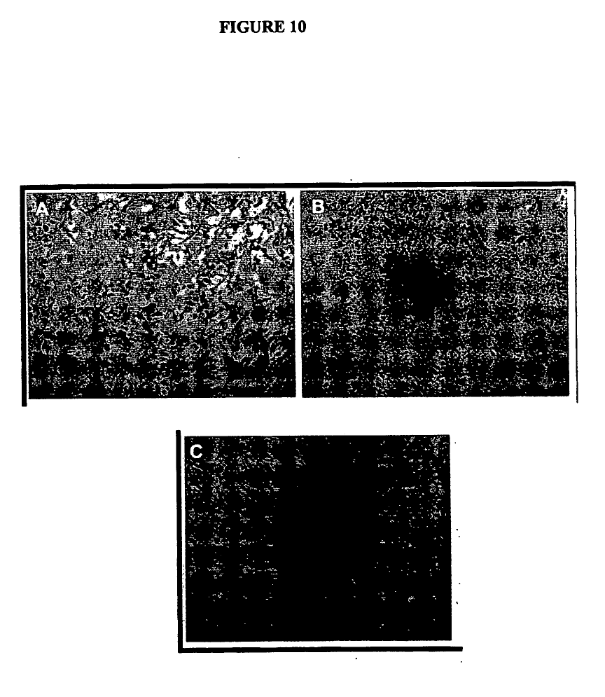

Cells (NIH3T3) were grown for 7 and 10 days in DMEM supplemented with 10% FBS, 1% penicillin/streptomycin (Gibco BRL), 50 mM L-ascorbic acid and 4 mM inorganic phosphate (Sigma). At days 7 and 10 von kossa staining for mineralization was performed. Control (non-transfected) NIH 3T3 cells showed no mineral deposits (FIG. 9A). Cells expressing rPP showed extensive mineral deposition (FIG. 9B). Microscopy images (FIGS. 10A, B and C) show von kossa stained control NIH3T3 cells (panel A) and transfected NIH3T3 cells (panels B and C). Panels B and C show mineral deposits and positive for von kossa staining whereas the control cells are negative for von kossa staining.

This example demonstrated that PP treatment of cells caused mineral deposition.

### Example 9

This example demonstrates the mineral formed by PP.

X-ray diffraction analysis was performed on NIH3T3 cells stably transfected with PP and cultured for 10 days. The cells were rinsed with water and then air dried. The dried cells/matrix/mineral was then collected and analyzed by x-ray diffraction using a X'PERT-Pro Philips x-ray diffractometor.

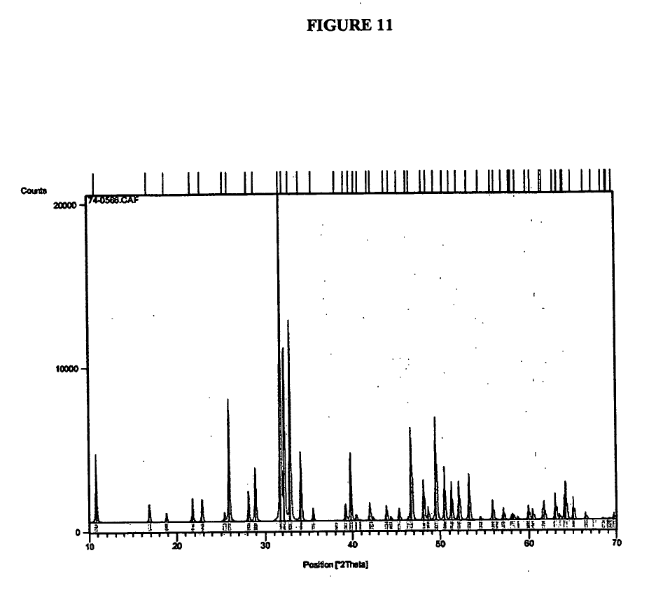

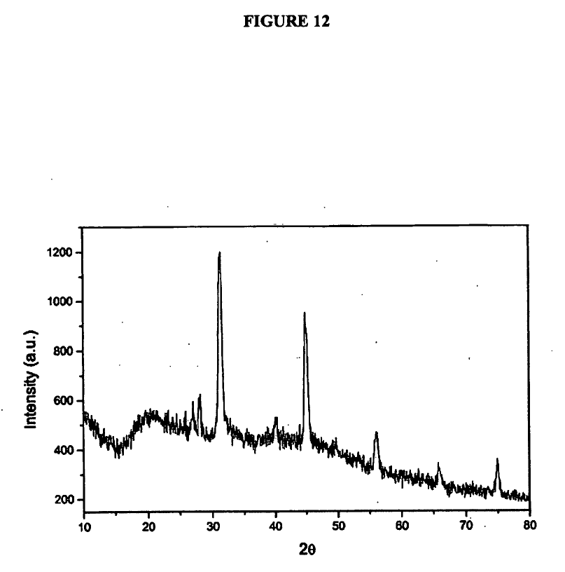

FIG. 11 displays the simulated x-ray pattern for stoichiometric Hydroxyapatite (HA). FIG. 12 shows the x-ray diffraction obtained from the experimental samples isolated from the PP transfected cells. As shown, the most intense peak at 20 angle of 31° matches very well with the most intense peak of the simulated hydroxyapatite pattern shown in FIG. 11. Moreover, the other peaks also appear to be closely correlated with the simulated HA pattern, which suggests that the mineral formed has its origin related to the apatite family. Such patterns with closely matching peaks are indicative of either a defective or anisotropic crystallographic orientation of the mineralized crystal.

This example demonstrated that PP caused the formation of hydroxyapatite, the main component of the mineral matrix of the tubule walls of dentin.

### Example 10

This example demonstrates that PP synergizes with BMP in biomineralization and osteoblastic differentiation.

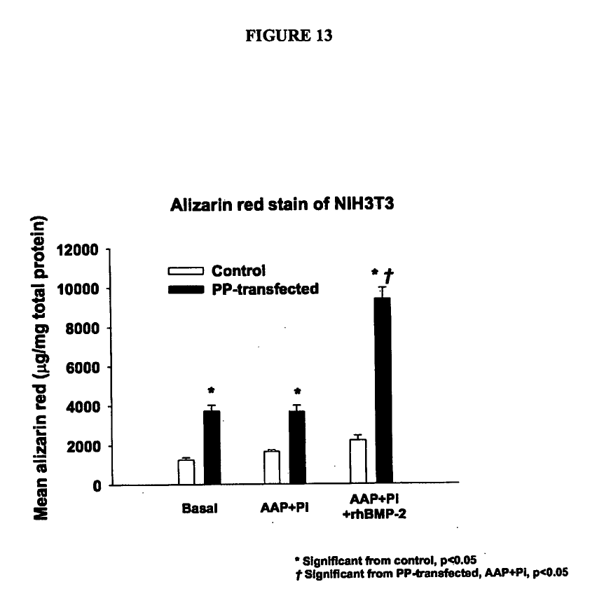

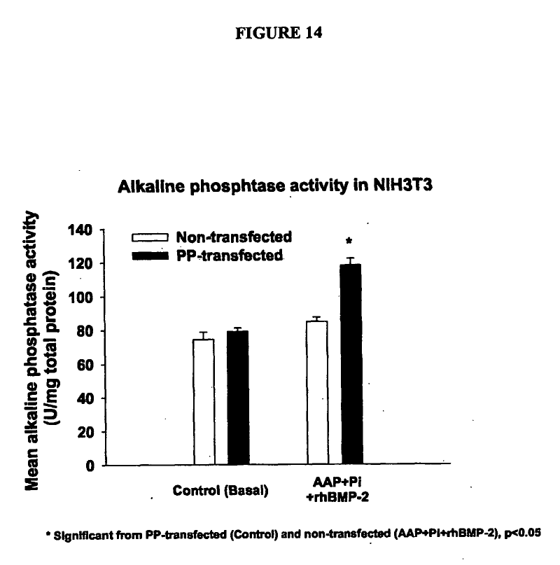

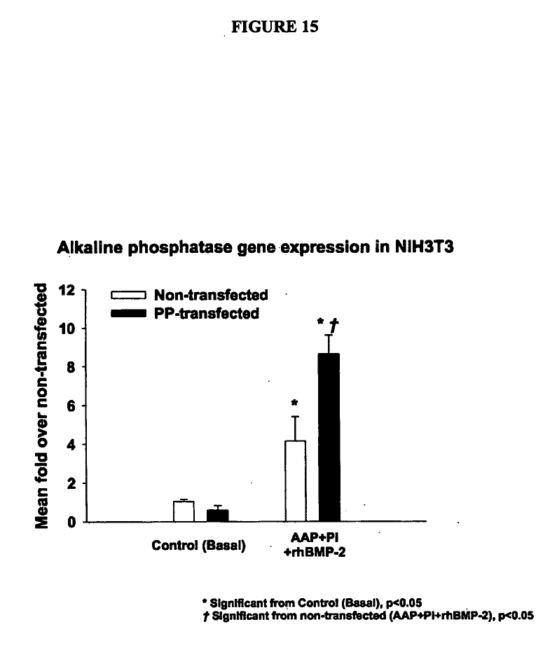

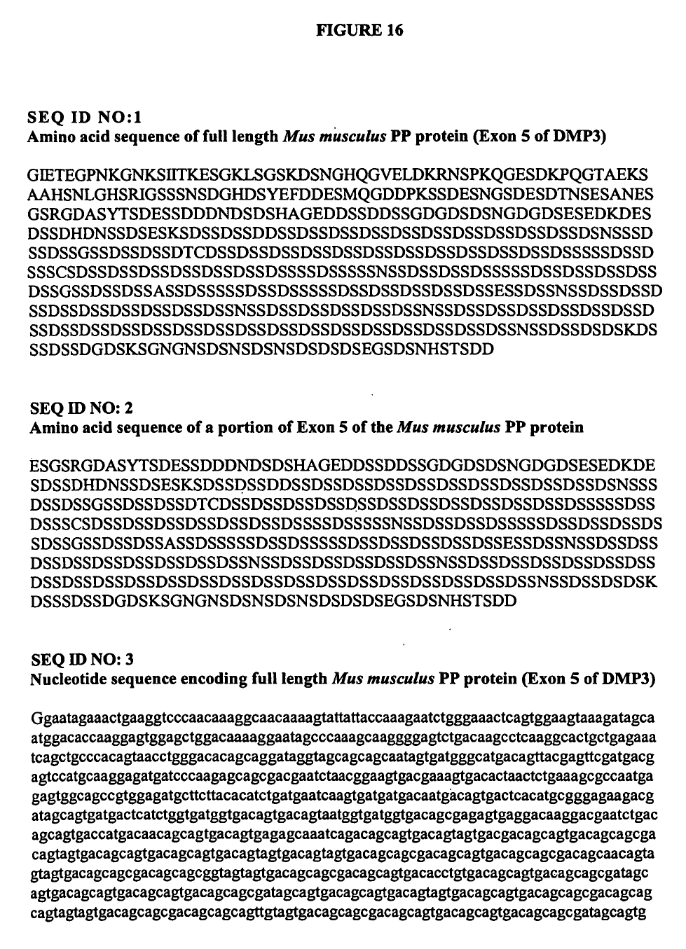

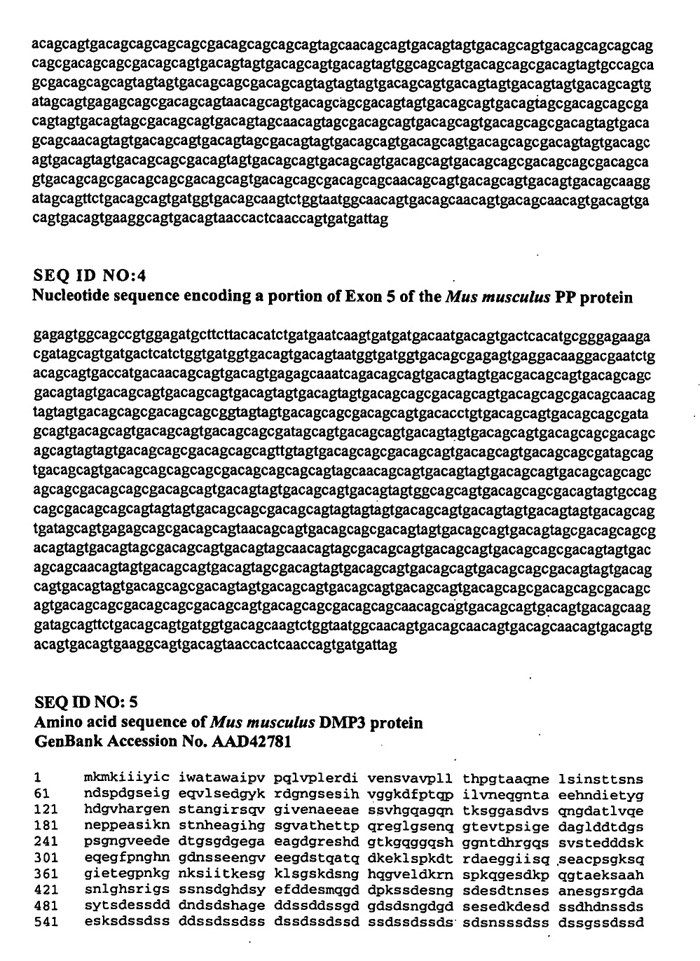

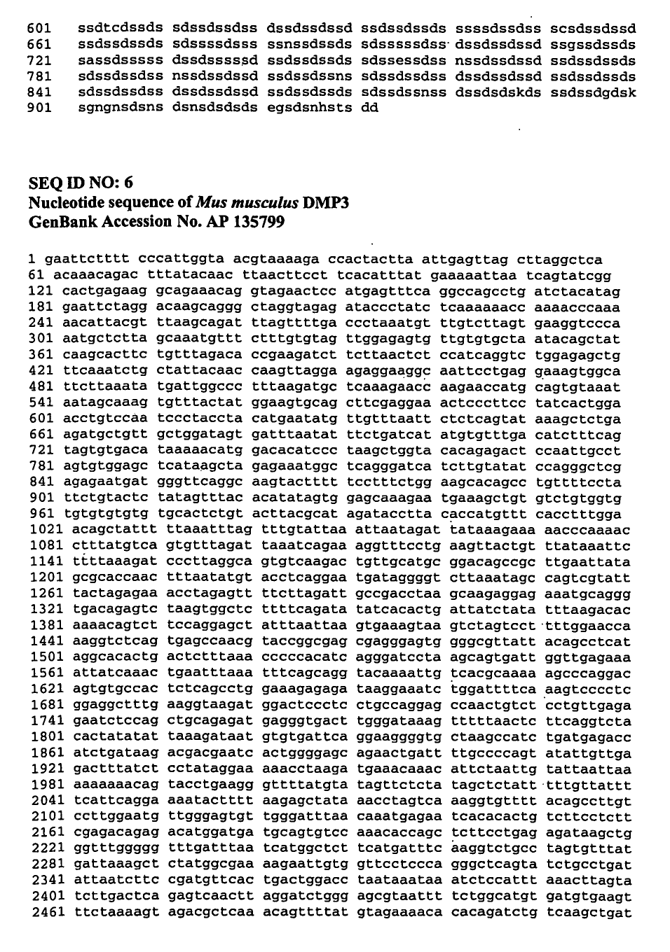

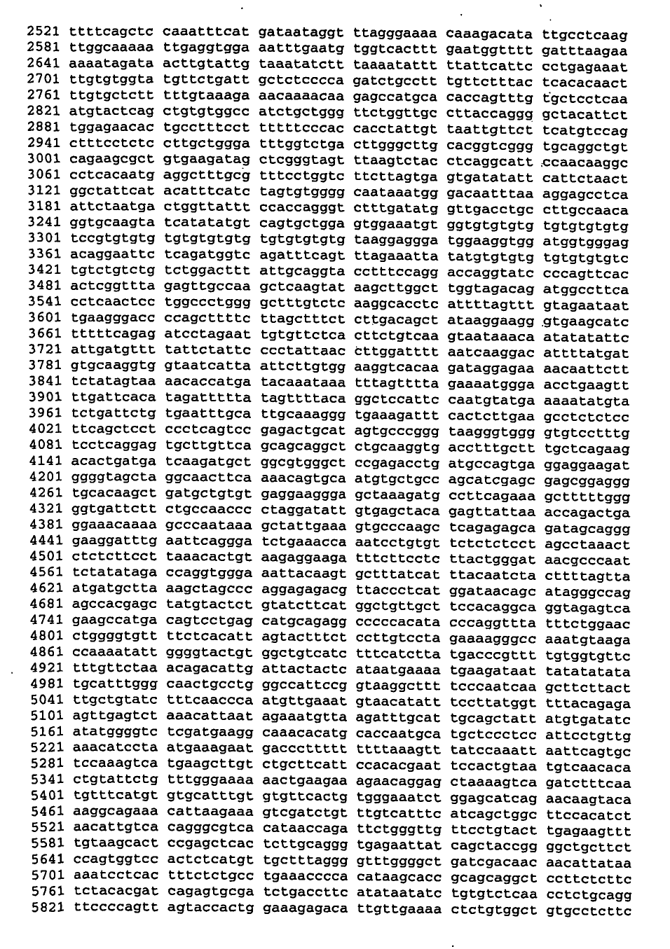

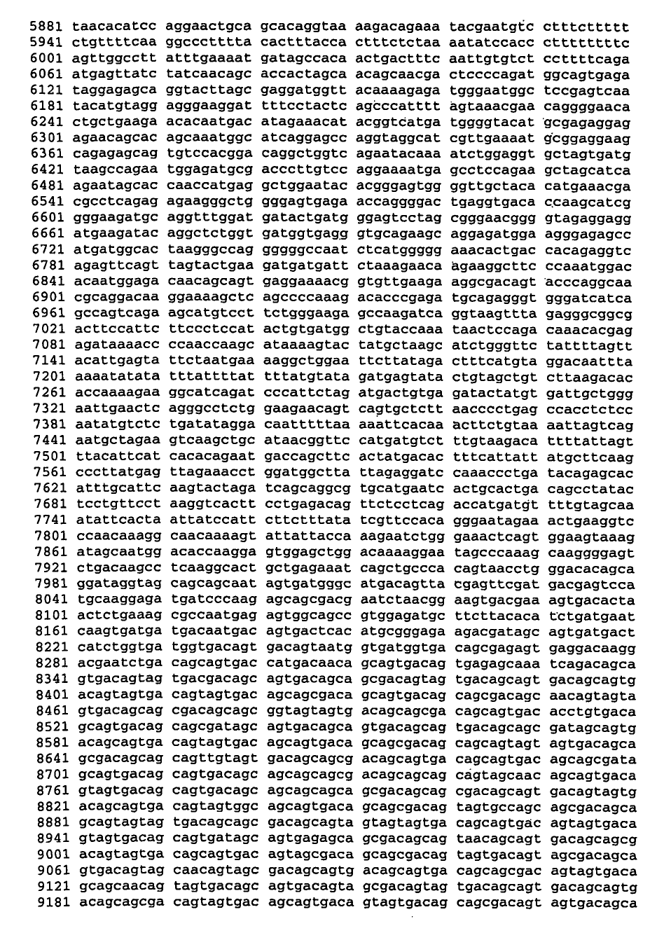

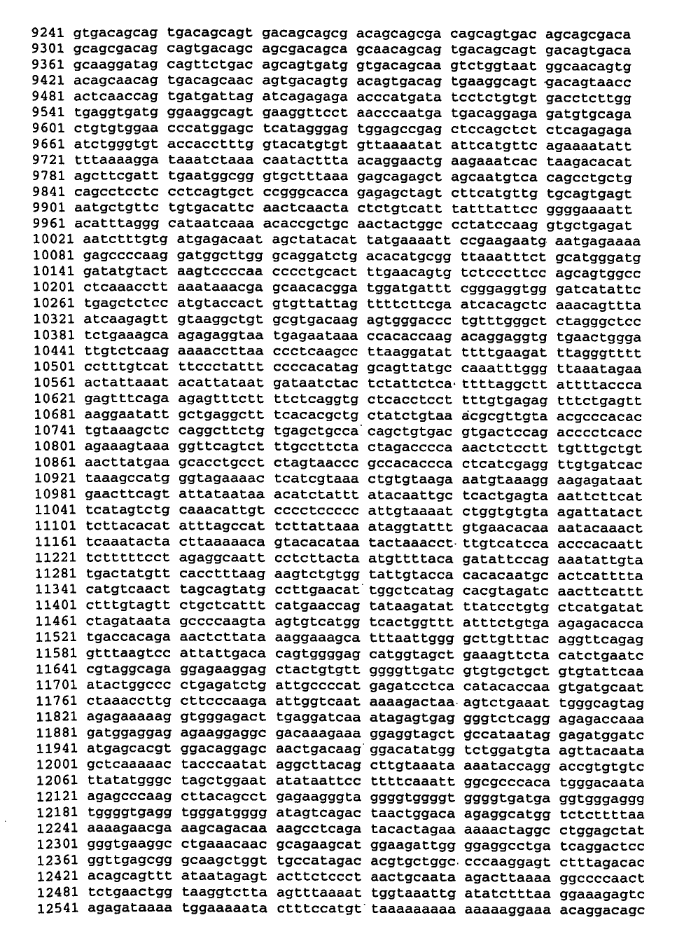

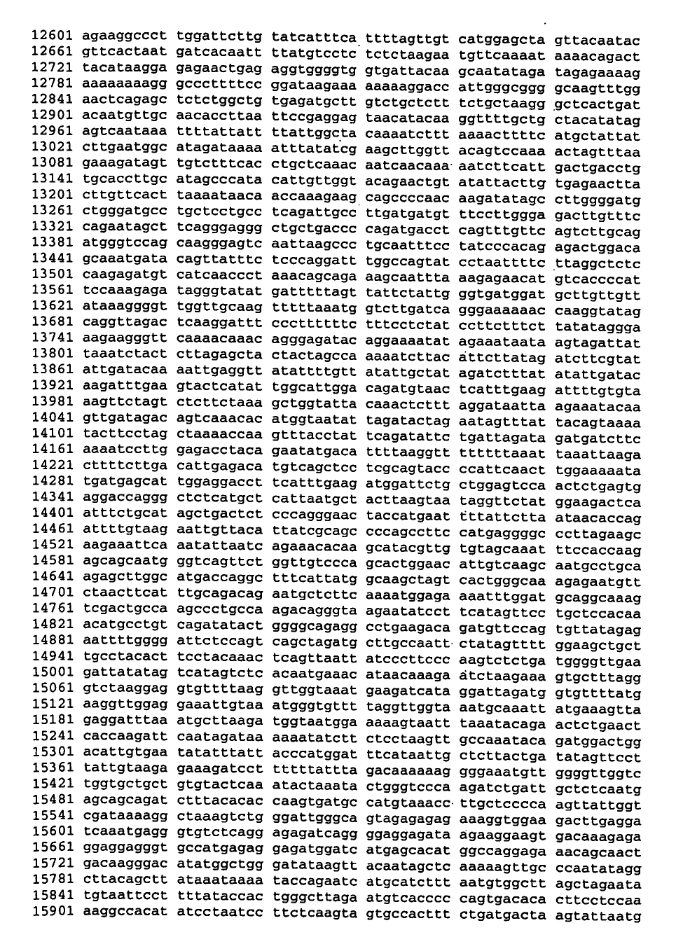

PP and BMP-2 when added in combination to NIH3T3 cells in culture deposit significantly more calcium than either PP or BMP-2 treated cells alone, as demonstrated by alizarin red staining (FIG. 13). Moreover, alkaline phosphatase activity is enhanced in PP-transfected NIH3T3 cells when additionally supplemented with BMP-2 (FIGS. 14 and 15). Values are significant over individual additive values of PP and BMP-2 alone.

This example demonstrated that PP synergistically acted with BMP-2 to deposit calcium.

### Example 11

This example demonstrates that cells expressing PP cause biomineralization.

Several samples of NIH3T3 and MC3T3 (wild type) and cells stably transfected with the PP gene (NIH3T3-PP and MC3T3-PP) were cultured for 21 days and assessed for their matrix mineralization using primarily X-ray diffraction, Fourier transform infrared spectroscopy (FTIR), electron microscopy and energy dispersive X-ray analysis (EDAX). The 21 day cultured cells were washed with de-ionized water diluted with ammonia to prevent dissolution of any of the mineralized phases.

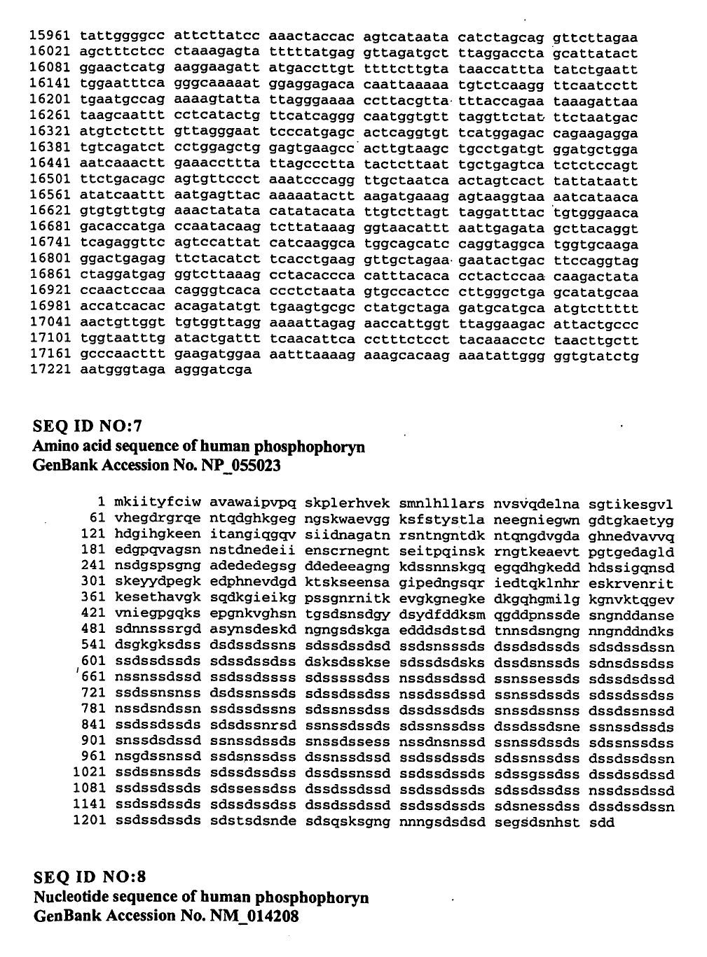

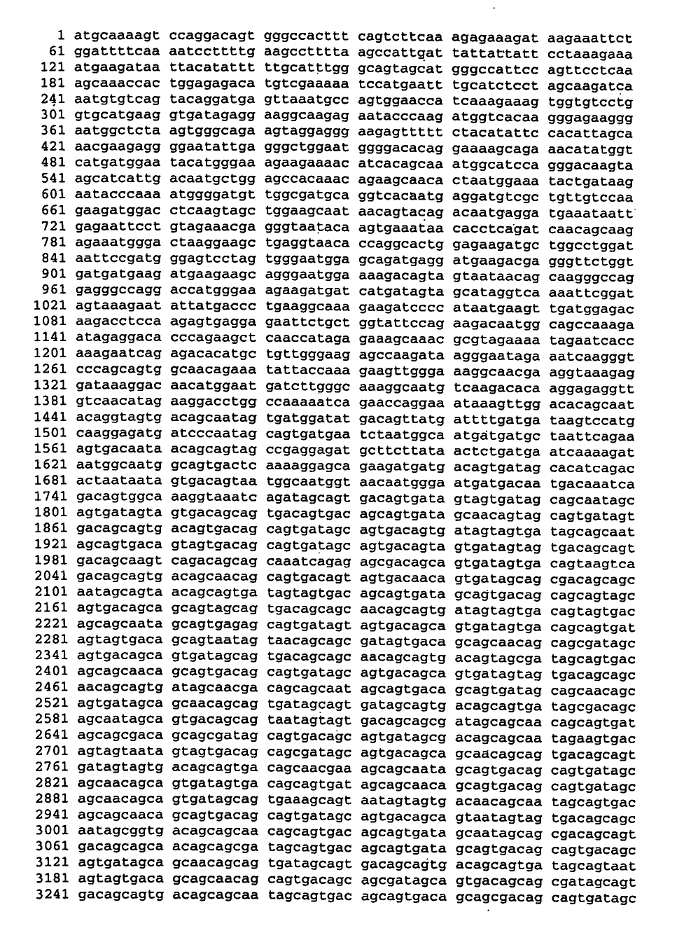

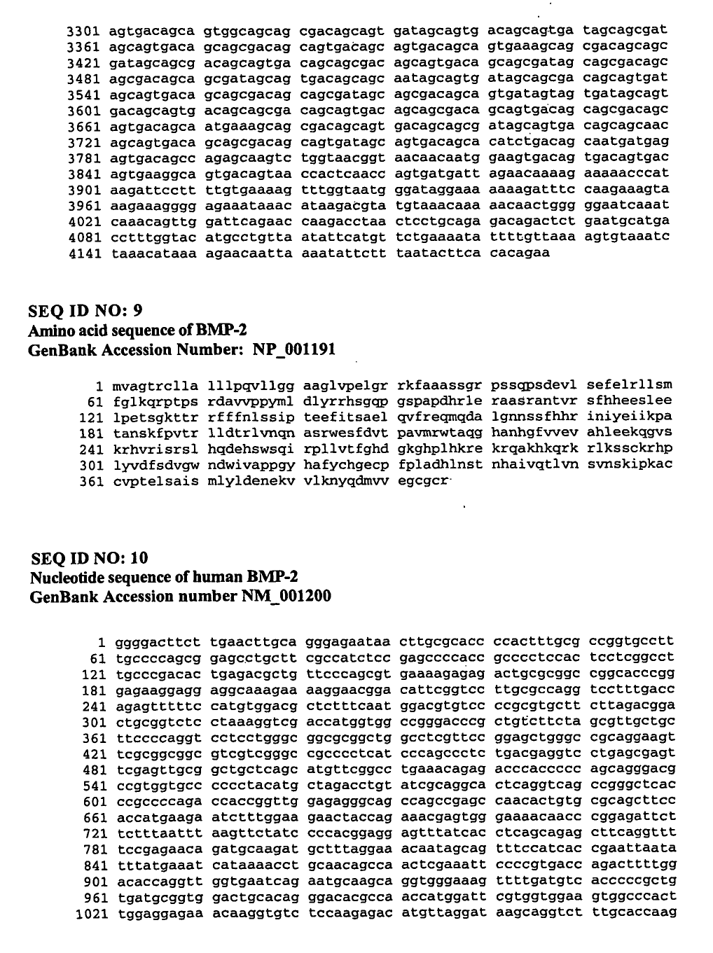

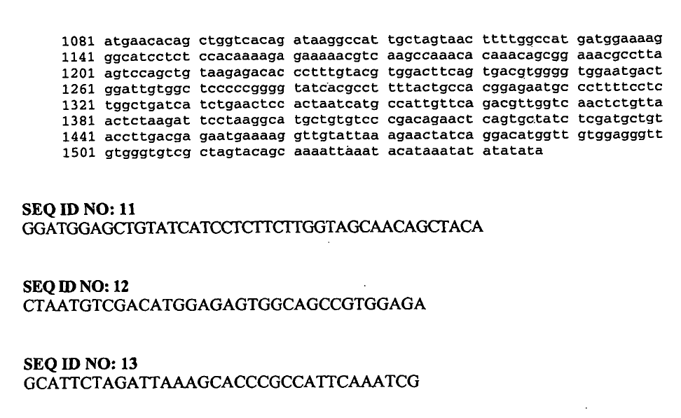

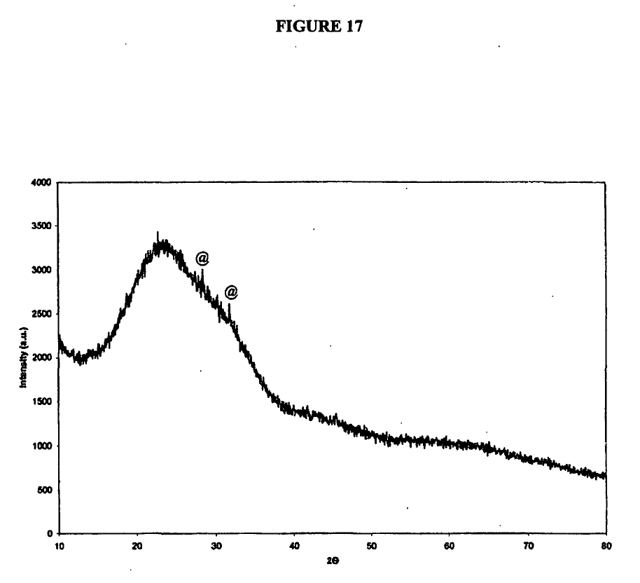

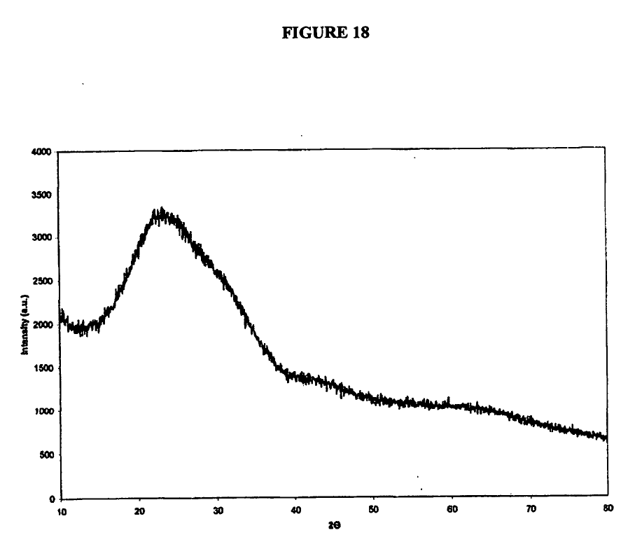

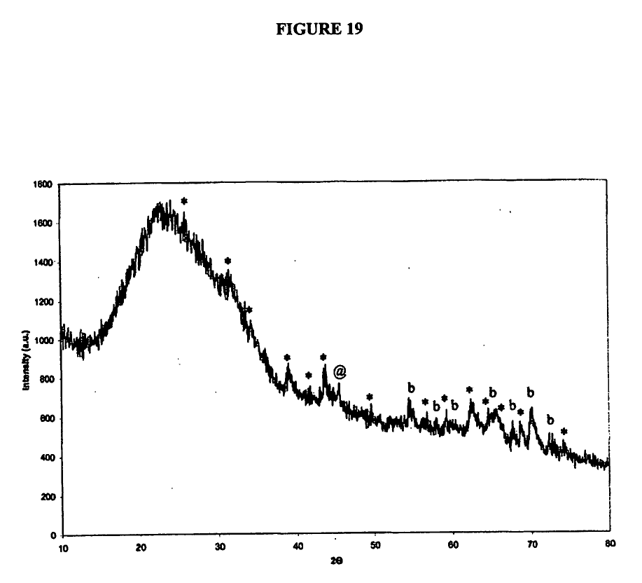

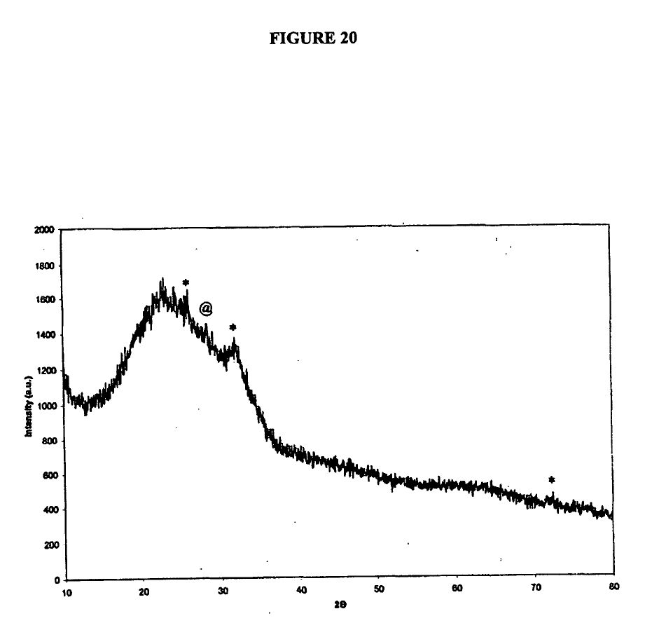

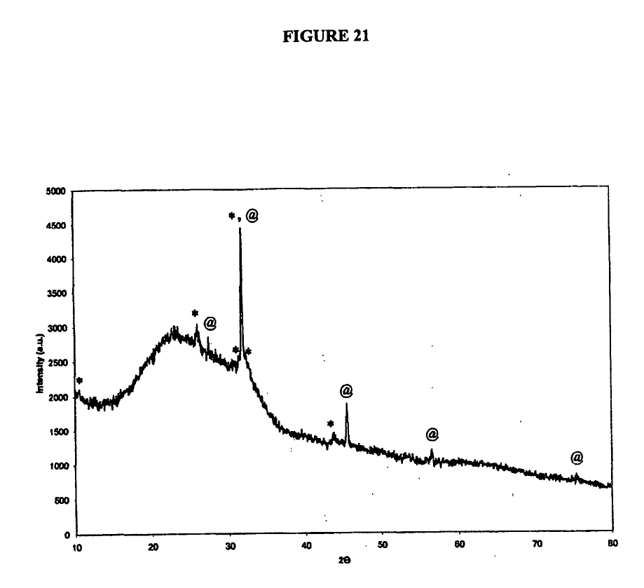

X-ray analysis allows the detection of calcium phosphate phases which indicates the possibility of hydroxyapatite (HA) and/or brushite formation. The X-ray analysis of cells, which were not genetically modified to secrete PP, demonstrated a lack of calcium phosphate mineral (FIGS. 17 and 18). This was expected, since NIH3T3 are fibroblastic cells, which do not normally mineralize. In contrast, the x-ray diffraction pattern of NIH3T3 cells expressing PP (NIH3T3-PP cells; FIG. 19) indicated the presence of brushite and hydroxyapatite in the extracellular space. The formation of brushite likely occurred due to the drop in pH of the media from about 7 to about 6. To test this theory, the buffering agent, HEPES, was added to the media in an effort to maintain a more stable and neutral pH. In the presence of HEPES, only HA was detected by x-ray diffraction (FIG. 20), thereby confirming the theory that the previous formation of brushite (FIG. 19) was due to the lowered pH of the medium. Furthermore, the X-ray diffraction of MC3T3 cells expressing PP also indicated that PP caused HA formation (FIG. 21).

### Example 12

This example demonstrates that PP induces the activation of Smad signaling.

The activation of the Smad pathway was investigated by treating human mesenchymal stem cells with rPP and assessing the phosphorylation state, which correlates with the activation state of Smad, by standard Western blot analysis. A phospho-Smad1 antibody (Santa Cruz Biotechnology, Cat# sc-12353), which recognized the dual phosphorylated serines corresponding to amino acids 463 and 465 at the C-terminal end of Smad1, was used to determine the phosphorylation state of Smad1 following the addition of rPP.

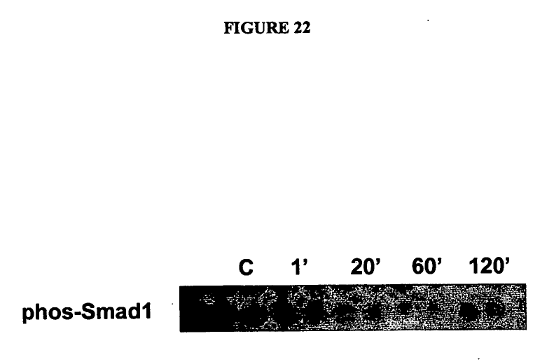

As shown in FIG. 22, Smad1 dual phosphorylation was apparent within 1 minute of exposure to rPP in hMSC cells. Interestingly, the addition of bone morphogentic proteins (BMP) also causes the phosphorylation of Smad at these sites. Reports in the literature have described that the Smad1 could also be phosphorylated at the linker region through the ERK MAP kinase and this phosphorylation at the linker region was shown to inhibit the BMP activity (Kretzschmar et al., Nature 389: 618-622 (1997); Massague et al., Genes Dev. 17: 2993-2997 (2003)).

This example showed that the activation of Smad1 could be induced by PP.

### Example 13

This example demonstrates that PP-/integrin-mediated signaling involves ILK.

Upon cell adhesion to the extracellular matrix, cells recruit a highly selective group of membrane and cytoplasmic proteins to the cell-extracellular matrix contact sites, where they connect the extracellular matrix to the actin cytoskeleton and regulate cell shape change, migration and signal transduction (Hynes et al., Cell 69: 11-25 (1992); Calderwood et al., J Biol. Chem. 275: 22607-22610 (2000); Jockusch et al., Ann. Rev. Cell. Dev. Biol. 11: 379-416 (1995); Yamada et al., Curr. Opn. Cell. Biol. 7:681-689 (1995); and Zamir et al., J. Cell. Sci. 112 (Pt 11): 1655-1669 (1999). Integrin Linked Kinase (ILK) has been shown to be involved in the regulation of a number of integrin-mediated processes that include cell adhesion, cell shape changes, gene expression, and ECM deposition (Wu et al., J. Biol. Chem. 273: 528-536 (1998). Since PP has been shown herein to be involved in integrin signaling, it was hypothesized that ILK is involved in this pathway.

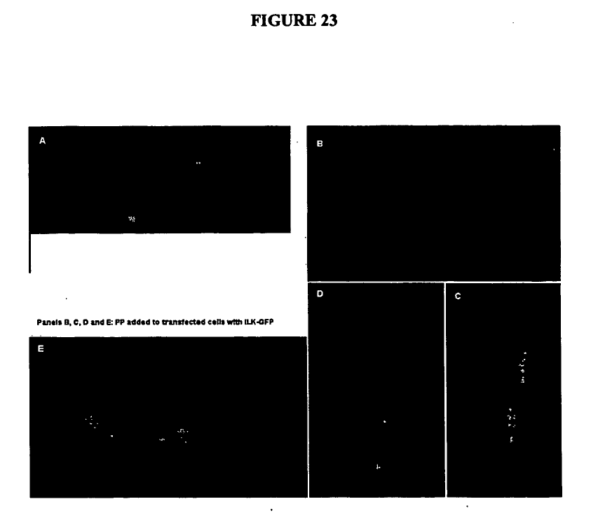

To determine whether or not ILK is involved in the PP-integrin signaling pathway, the following was performed: A clone coding for a GFP-ILK fusion protein was provided by Dr. Carry Wu from the University of Pittsburgh. DNA fragments encoding wild-type ILK was cloned into the EcoRI/SalI sites of the pEGFP-C2 vector (Clontech) as described by Zhang Y. et al. (Zhang et al., J. Cell. Sci. 115: 4777-4786 (2002)). Tissue culture wells were coated with Poly-L-Lysine, a nonspecific adhesion-promoting polypeptide Ishida et al., Biochem Biophys Res Commun 300: 201-208 (2003); and Chen et al., J Biol Chem 269: 26602-26605 (1994)), which does not seem to activate integrin receptors. MDPC-23 cells were then plated in serum free media and transfected with the ILK-GFP construct using LipoFectamine Plus (Life Technologies). In one well, PP (250 ng/ml of media) was added, whereas the control did not receive any PP. Following a 6 hour incubation, pictures were acquired using a fluorescence microscope. As shown in FIG. 23, the addition of PP caused a clustering of fluorescence, which indicated that the ILK-GFP protein was involved in rPP-integrin signaling. It further indicated that ILK-GFP was involved in the formation of focal adhesion sites.

This example further confirms that PP signals with integrins and that the PP/integrin-mediated signaling involves ILK.

### Example 14

This example demonstrates the synthesis of nanostructured hydroxyapatite (HA), brushite, tri-calcium phosphate (TCP), and amorphous calcium phosphates corresponding to HA and TCP compositions, including generation of supports containing natural and synthetic polymers and PP.

Materials used for HA synthesis: Calcium chloride (Aldrich, 99% ACS grade), tri-sodium phosphate, 96% Aldrich, De-ionized water (NANO Pure, 18.2 MΩ-cm), NaOH (97% min. anhydrous pellet, ACS, Alfa Aesar).

Approach: In a typical synthesis, HA was synthesized using commercially obtained anhydrous CaCl₂ (99% A.C.S., Aldrich) and Na₃PO₄ (96%, Aldrich) as starting precursors. The reaction was conducted using de-ionized water (NANO pure, MΩ-cm 18.2) according to the following reaction:
10CaCl₂+6Na₃PO₄+xNaOH→Ca_{9+x/2}(PO₄)₆(OH)₂+(18+x)NaCl+(1−x/2)CaCl₂(x=0,1,2)

The method consisted of the following steps: Stoichiometric amount of Na₃PO₄ (0.12M) was first dissolved in water and various amounts (x=0,1, 2) of NaOH (97% min. anhydrous pellet, ACS, Alfa Aesar) were added to the solution before reacting with stoichiometric amounts of CaCl₂ (0.2M) to control the stoichiometry and thermal stability of HA. The addition of stoichiometric amounts of HA, x=2 resulted in stoichiometric HA. The reaction was allowed to proceed for 24 h in air. After 24 h, the HA powders were centrifuged and washed five times with de-ionized water, in order to remove all of the NaCl formed as a by-product during the reaction. The collected HA powder was then dried at 80° C. for 24 h. The synthesized HA powders comprised of crystallites in the size range of 10-20 nm. The methodology was similar to that described in U.S. Patent Publication No. 2003-0219466, filed Mar. 19, 2003.

Generation of polymer-HA composites containing PP: The synthesized HA powders were incorporated into water soluble polymers, such as Polyethylene glycol (PEG) and agarose and alginate gels, by dispersing the powders in the dissolved polymer after which evaporation of the solvent resulted in the formation of the composite sheet similar to the approaches described in U.S. Patent Publication No. 2003-0219466. PP was incorporated into the dissolved polymer blend. In the case of non-water soluble polymers, the synthesized HA and PP were dispersed in the solubilized polymer. In the case of PCL, PLA and PLGA, the preferred solvent was THF or methylene chloride. The approach to generate the composite was essentially the same, wherein the synthesized nanosized HA powders were dispersed in the dissolved polymer containing the PP protein. Evaporation of the solvent provided and composited structure. Alternatively, PP was introduced after the substrate was formed as well by casting the blends in a tissue culture plate into which the PP protein was added. The polymer-ceramic composites also were cast into a paste or a cream following approaches similar to those mentioned earlier.

Materials and Synthesis of Unsubtituted and Substituted Brushite:

Undoped and Doped Brushite

Materials for brushite synthesis: Na₂HPO₄ (ACS reagent grade, anhydrous, ACROS), CaCl₂.2H₂O (ACS reagent grade, ACROS), de-ionized water (NANO Pure, 18.2 MΩ-cm), MgCl₂.6H₂O (99%, ACROS).

Approach: The synthesis of pure undoped brushite was based on the following chemical reaction:
Na₂HPO₄+CaCl₂+2H₂O→CaHPO₄.2H₂O+2NaCl

Na₂HPO₄ (0.05 mol, ACS reagent grade, anhydrous, ACROS) was dissolved in 100 ml of distilled water. Also, CaCl₂.2H₂O (0.05 mol, ACS reagent grade, ACROS) was dissolved in 100 ml distilled water. Both solutions were stirred until the salts were completely dissolved. After the solutions were prepared, the CaCl₂.2H₂O solution was added dropwise to Na₂HPO₄ solution while stirring. The precipitate was then centrifuged, washed with distilled water, and dried at 60° C. in drying-oven overnight.

The synthesis of Mg substituted brushite was based on the following chemical reaction (14% Mg/Ca ratio was used for the chemical reaction):
Na₂HPO₄+0.86CaCl₂+0.14MgCl₂+2H₂O→(Ca_{0.86}Mg_{0.14})HPO₄.2H₂O+2NaCl
MgCl₂.6H₂O (0.007 mol, 99%, ACROS) and 0.043 mol of CaCl₂.2H₂O (ACS reagent grade, ACROS) were dissolved in 100 ml of distilled water. Na₂HPO₄ (0.05 mol, ACS reagent grade, anhydrous, ACROS) also was dissolved in 100 ml of distilled water. Both solutions were stirred until the salts were completely dissolved. Mg/Ca solution (14%) was then added dropwise to the Na₂HPO₄ solution while stirring. The precipitate was then centrifuged, washed with distilled water, and dried at 60° C. in drying-oven overnight.

Generation of polymer-brushite composites containing PP: The synthesized brushite powders were incorporated into water soluble polymers, such as Polyethylene glycol (PEG) and agarose and alginate gels by dispersing the powders in the dissolved polymer after which evaporation of the solvent resulted in the formation of the composite sheet. PP was incorporated into the dissolved polymer blend. In the case of non-water soluble polymers, the synthesized brushite and PP were dispersed in the solubilized polymer. In the case of PCL, PLA and PLGA, the preferred solvent was THF or methylene chloride. The approach to generate the composite were essentially the same wherein the synthesized nanosized brushite powders were dispersed in the dissolved polymer containing the PP protein. Evaporation of the solvent provided and composited structure. Alternatively, PP was introduced after the substrate was formed as well by casting the blends in a tissue culture plate into which the PP protein is added. The polymer-ceramic composites also were cast into a paste or a cream following approaches similar to those mentioned earlier.

Materials and Synthesis of TCP:

Materials for TCP Synthesis: CaCl₂.2H₂O (ACS reagent grade, ACROS), MgCl₂-6H₂O (99%, ACROS), de-ionized distilled water, (NANO Pure, 18.2 MΩ-cm), Na₂HPO₄ (ACS reagent grade, anhydrous, ACROS).

Approach:

Tricalcium Phoshate (TCP): TCP was synthesized by a two-step approach. The first step involved synthesizing a Mg-substituted brushite phase which was then subjected to a slow in situ hydrolysis step to form Mg-substituted TCP, called TCMP.

Mg-Substituted Brushite

The synthesis of magnesium substituted brushite was based on the following chemical reaction (Mg/Ca=1 ratio was used for the reaction):
2Na₂HPO₄+CaCl₂.2H₂O+MgCl₂.6H₂O→2(Ca_{0.5}Mg_{0.5})HPO₄.2H₂O+4NaCl+4H₂O

CaCl₂.2H₂O (0.025 mol, ACS reagent grade, ACROS) and 0.025 mol of MgCl₂.6H₂O (99%, ACROS) were simultaneously dissolved in 100 ml of distilled water. Na₂HPO₄ (0.05 mol, ACS reagent grade, anhydrous, ACROS) also was dissolved in 100 ml of distilled water. Both solutions were stirred until the salts were completely dissolved. 50% Mg/Ca solution was then added to the Na₂HPO₄ solution with the addition rate of ˜4 ml/sec, using the Fisherbrand Variable-Flow Chemical Transfer Pump, while stirring. The precipitate was then centrifuged, washed, and dried at 60° C. in drying-oven overnight.

Mg Substituted TCP(β-TCMP)

β-TCMP was synthesized using an in-situ growth technique. The 50% magnesium substituted brushite powder was used as a precursor for β-TCMP. Magnesium substituted brushite powder was dispersed in 200 ml of distilled water, and then transferred to a vial with an attached condenser. It was then boiled for 8 h, 4 h, 1 h, 30 min and 15 min to find the minimum time required for boiling. The powder was then collected each time after boiling and washed with distilled water. They were dried at 60° C. in a drying-oven overnight after being collected.

Generation of Polymer-TCP Composites Containing PP:

The synthesized TCP powders were incorporated into water soluble polymers such as Polyethylene glycol (PEG) and agarose and alginate gels by dispersing the powders in the dissolved polymer after which evaporation of the solvent resulted in the formation of the composite sheet. PP was incorporated into the dissolved polymer blend. In the case of non-water soluble polymers, the synthesized TCP and PP were dispersed in the solubilized polymer. In the case of PCL, PLA and PLGA, the preferred solvent was THF or methylene chloride. The approach to generate the composite was essentially the same wherein the synthesized nanosized TCP powders were dispersed in the dissolved polymer containing the PP protein. Evaporation of the solvent provided and composited structure. Alternatively, PP was introduced after the substrate was formed as well by casting the blends in a tissue culture plate into which the PP protein was added. The polymer-ceramic composites also were cast into a paste or a cream following approaches similar to those mentioned earlier.

Materials and Synthesis of Amorphous Calcium Phosphates Corresponding to HA and TCP Compositions:

Materials: Na₃PO₄.12H₂O (ACS reagent grade, ACROS), NaOH (pellets, 98%, ACROS), deionized distilled water (conductivity 18.3 MΩ), MgCl₂-6H₂O (99%, ACROS) and CaCl₂.2H₂O (ACS reagent grade, ACROS).

Approaches: The synthesis of ACP corresponding to HA composition was based on the following chemical reaction.
7CaCl₂.2H₂O+3MgCl₂.6H₂O+6Na₃PO₄.12H₂O+2NaOH→Ca₇Mg₃(PO₄)₆.(OH)₂+20NaCl+104H₂O
Na₃PO₄.12H₂O (0.06 moles, ACS reagent grade, ACROS) and 0.02 moles of NaOH (pellets, 98%, ACROS) were dissolved in 100 ml deionized distilled water (conductivity 18.3 MΩ). Also, 0.03 moles of MgCl₂-6H₂O (99%, ACROS) and 0.07 moles of CaCl₂-2H₂O (ACS reagent grade, ACROS) were dissolved in 100 ml deionized distilled water (conductivity 18.3 MΩ). Both solutions were stirred until the chemicals were completely dissolved. Mg/Ca solution was then added dropwise (added with 40 ml syringe by hand) to Na₃PO₄.12H₂O solution while stirring, although the addition rate was not important. The precipitation was then centrifuged, washed with deionized distilled water (conductivity 18.3 MΩ), and dried at 60° C. in a drying-oven.

The synthesis of ACP corresponding to the TCP composition was based on the following chemical reaction.
2.4CaCl₂.2H₂O+0.6MgCl₂.6H₂O+2Na₃PO₄.12H₂O Ca_{2.4}Mg_{0.6}(PO₄)+6NaCl+32.4H₂O
Na₃PO₄.12H₂O (0.02 moles, ACS reagent grade, ACROS) was dissolved in 100 ml deionized distilled water (conductivity 18.3 MΩ). Also, 0.006 moles of MgCl₂.6H₂O (99%, ACROS) and 0.024 moles of CaCl₂.2H₂O (ACS reagent grade, ACROS) were dissolved in 100 ml deionized distilled water (conductivity 18.3 MΩ). Both solutions were stirred until the chemicals were completely dissolved. Mg/Ca solution was then added dropwise (added with 40 ml syringe by hand) to Na₃PO₄ 12H₂O solution while stirring, although the addition rate was not observed to be important. The precipitate was then centrifuged, washed with deionized distilled water (conductivity 18.3 MΩ), and dried at 60° C. in a drying-oven.

Generation of Polymer-ACP Composites Containing PP:

The synthesized ACP powders were incorporated into water soluble polymers such as Polyethylene glycol (PEG) and agarose and alginate gels by dispersing the powders in the dissolved polymer after which evaporation of the solvent resulted in the formation of the composite sheet. PP was incorporated into the dissolved polymer blend. In the case of non-water soluble polymers, the synthesized ACP and PP were dispersed in the solubilized polymer. In the case of PCL, PLA and PLGA, the preferred solvent was THF or methylene chloride. The approach to generate the composite was essentially the same wherein the synthesized nanosized ACP powders were dispersed in the dissolved polymer containing the PP protein. Evaporation of the solvent provided and composited structure. Alternatively, PP was introduced after the substrate was formed as well by casting the blends in a tissue culture plate into which the PP protein was added. The polymer-ceramic composites also were cast into a paste or a cream following approaches similar to those mentioned earlier.

### Example 15

This example illustrates the approach used for synthesizing calcium phosphate gels corresponding to different Ca/P ratios.

Materials: Ca(NO₃)₂.4H₂O (0.01 mole, Acros, 99%) and P₂O₅ (0.003 mol, Acros, 99%) and ethyl alcohol (AAper, Shelbyville, 190 proof)

Approach: In a typical synthesis approach for generating calcium phosphate gels, commercially obtained Ca(NO₃)₂.4H₂O (0.01 mole, Acros, 99%) and P₂O₅ (0.003 mol, Acros, 99%) were used with the molar ratio of 10:3, which ws the desired Ca/P ratio observed in hydroxyapatite, and 5, 10 and 15 ml of ethyl alcohol (AAper, Shelbyville, 190 proof) was used as the solvent as received without conducting any additional purification treatments. The Ca/P ratio was altered to generate gels of varying Ca and P compositions. Thus, gels corresponding to brushite, TCP and OCP were generated. The sequence of the dissolution of the above precursors was not important although the rapid addition of a solution of one precursor to the other precursor resulted in precipitation. The as-prepared solution after dissolving the above precursors slowly transformed into a gel after a period of 30 min to a maximum of 2h after termination of the initial stirring of the solution for 30 min. Exchange of alcohol with water was initiated by immersing the gel in deionized water and washing the gel to result in a solvent exchanged gel called aquagel. The nitrate salt also was replaced with CaCl₂ that is more biocompatible than the nitrate salt.

### Example 16

This example demonstrates a method of testing PP's ability to cause periodontal regeneration or cementum formation.

Cementum formation, or cementogenesis, is important in reconstructing periodontal structures. Since the data described herein demonstrate that PP can induce the regeneration of bone and dentin tissue, it was hypothesized that PP could also induce the regeneration of periodontal tissue through the formation of cementum.

The affect of PP on cementogenesis is tested by the methods described in Giannobile et al., J. Periodontol. 72(6): 815-823 (2001). Briefly, recombinant adenoviral vectors encoding the PP gene are constructed to allow delivery of PP transgenes to cells. The recombinant adenoviruses are assembled using the viral backbone of Ad2/CMV/EGFP and replacing GFP (reporter gene encoding green fluorescent protein driven by the cytomegalovirus promoter (CMV) within adenovirus type 2) with the PP gene. Root lining cells (cloned cementoblasts) are transduced with Ad2/PP and evaluated for gene expression, DNA synthesis, and cell proliferation. PP-inducible genes are also evaluated following gene delivery of Ad2/PP.

This example demonstrates a method of assaying PP's ability to induce periodontal regeneration.

### Example 17

This example demonstrates a method of testing the effectiveness of PP treatment in humans.

The ability of PP to regenerate periodontal tissue in humans is tested following the procedures described in Howell et al., J Periodontol 68(12): 1186-1193 (1997). Briefly, thirty-eight human subjects possessing bilateral osseous periodontal lesions are assigned to one of two treatment groups in a split-mouth design. Following full-thickness flap reflection, test sites receive local application of the therapeutic drug (i.e., PP) delivered in coded syringes by a “masked” investigator. Two dose levels of rhPP in a gel vehicle are tested. Control treatment consists of either conventional periodontal flap surgery or surgery plus vehicle. Safety analyses includes physical examination, hematology, serum chemistry, urinalysis, antibody titers, and radiographic evaluation of bony changes. The primary therapeutic assessment is bone fill measured at re-entry 6 to 9 months after treatment.

This example demonstrated a method of testing the ability of PP to regenerate periodontal tissue in humans.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of inducing biomineralization in a tissue, which method comprises administering to the tissue a source of Phosphophoryn (PP) in an amount sufficient to induce biomineralization in the tissue.

2. The method of claim 1, wherein the source of PP is a PP protein having or comprising the amino acid sequence of SEQ ID NO: 1, a fragment thereof, or a derivative of either of the foregoing.

3. The method of claim 2, wherein the fragment of a PP protein has or comprises the amino acid sequence of SEQ ID NO: 2.

4. The method of claim 1, wherein the source of PP is a nucleic acid molecule encoding a PP protein (SEQ ID NO: 1), a fragment thereof, or a derivative of either of the foregoing, wherein the nucleic acid molecule is optionally in the form of an expression vector.

5. The method of claim 4, wherein the nucleic acid molecule encoding the PP protein has or comprises the nucleotide sequence of SEQ ID NO: 3.

6. The method of claim 4, wherein the nucleic acid molecule encoding the PP protein has or comprises the nucleotide sequence of SEQ ID NO: 4.

7. The method of claim 4, wherein the tissue is pulp tissue and the nucleic acid molecule encoding a PP protein, a fragment thereof, or a derivative of either of the foregoing is administered to a cell of the pulp tissue.

8. The method of claim 1, wherein the tissue is in a mammal.

9. The method of claim 8, wherein the mammal is a human.

10. The method of claim 1, wherein the source of PP is administered in combination with another osteogenic factor or a growth factor.

11. The method of claim 10, wherein the other osteogenic factor or the growth factor is a Bone Morphogenic Protein (BMP), Latent Membrane Protein-3 (LMP-3), a Platelet-Derived Growth Factor (PDGF), an Insulin Growth Factor (IGF), a Vascular Endothelial Growth Factor (VEGF), RunX, Osterix (Osx), or a Fibroblast Growth Factor (FGF).

12. The method of claim 1, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer.

13. The method of claim 1, wherein the method treats tooth sensitivity or injured pulp tissue.

14. A method of treating tooth sensitivity or injured pulp tissue in a mammal, which method comprises administering to the mammal a source of PP in an amount sufficient to treat tooth sensitivity or injured pulp tissue.

15. The method of claim 14, wherein the source of PP is a PP protein having or comprising the amino acid sequence of SEQ ID NO: 1, a fragment thereof, or a derivative of either of the foregoing.

16. The method of claim 15, wherein the fragment of a PP protein has or comprises the amino acid sequence of SEQ ID NO: 2.

17. The method of claim 14, wherein the source of PP is a nucleic acid molecule encoding a PP protein (SEQ ID NO: 1), a fragment thereof, or a derivative of either of the foregoing, wherein the nucleic acid molecule is optionally in the form of an expression vector.

18. The method of claim 17, wherein the nucleic acid molecule encoding the PP protein has or comprises the nucleotide sequence of SEQ ID NO: 3.

19. The method of claim 17, wherein the nucleic acid molecule encoding the PP protein has or comprises the nucleotide sequence of SEQ ID NO: 4.

20. The method of claim 17, wherein the mammal has pulp tissue and the nucleic acid molecule encoding a PP protein, a fragment thereof, or a derivative of either of the foregoing is administered to a cell of the pulp tissue.

21. The method of claim 14, wherein the mammal is a human.

22. The method of claim 14, wherein the source of PP is administered in combination with another osteogenic factor or a growth factor.

23. The method of claim 22, wherein the other osteogenic factor or the growth factor is a BMP, LMP-3, a PDGF, an IGF, a VEGF, RunX, Osx, or an FGF.

24. The method of claim 14, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer, optionally in combination with a calcium phosphate.

25. A method of inducing differentiation of a cell into an osteogenic cell or an odontogenic cell, which method comprises administering to the cell a source of PP in an amount sufficient to induce differentiation of the cell into an osteogenic cell or an odontogenic cell.

26. The method of claim 25, wherein the source of PP is a PP protein having or comprising the amino acid sequence of SEQ ID NO: 1, a fragment thereof, or a derivative of either of the foregoing.

27. The method of claim 26, wherein the fragment of a PP protein has or comprises the amino acid sequence of SEQ ID NO: 2.

28. The method of claim 25, wherein the source of PP is a nucleic acid molecule encoding a PP protein (SEQ ID NO: 1), a fragment thereof, or a derivative of the foregoing, wherein the nucleic acid molecule is optionally in the form of an expression vector.

29. The method of claim 28, wherein the nucleic acid molecule encoding the PP protein has or comprises the nucleotide sequence of SEQ ID NO: 3.

30. The method of claim 28, wherein the nucleic acid molecule encoding the PP protein has or comprises the nucleotide sequence of SEQ ID NO: 4.

31. The method of claim 28, wherein the cell is a stem cell and the nucleic acid molecule encoding a PP protein, a fragment thereof, or a derivative of either of the foregoing is administered to the stem cell.

32. The method of claim 25, wherein the cell is in a mammal.

33. The method of claim 32, wherein the mammal is a human.

34. The method of claim 25, wherein the method effectively induces bone regeneration.

35. The method of claim 25, wherein the cell is a stem cell.

36. The method of claim 25, wherein the cell is a fibroblast cell.

37. The method of claim 25, wherein the source of PP is administered in combination with another osteogenic factor or a growth factor.

38. The method of claim 37, wherein the other osteogenic factor or the growth factor is a BMP, LMP-3, a PDGF, an IGF, a VEGF, RunX, Osx, or an FGF.

39. The method of claim 25, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer.

40. A method of inducing bone or dentin regeneration in a tissue, which method comprises administering to the tissue a source of PP in an amount sufficient to induce bone or dentin regeneration in the tissue.

41. The method of claim 40, wherein the source of PP is a PP protein having or comprising the amino acid sequence of SEQ ID NO: 1, a fragment thereof, or a derivative of either of the foregoing.

42. The method of claim 41, wherein the fragment of a PP protein has or comprises the amino acid sequence of SEQ ID NO: 2.

43. The method of claim 40, wherein the source of PP is a nucleic acid molecule encoding a PP protein (SEQ ID NO: 1), a fragment thereof, or a derivative of either of the foregoing; wherein the nucleic acid molecule is optionally in the form of an expression vector.

44. The method of claim 43, wherein the nucleic acid molecule encoding the PP protein has or comprises the nucleotide sequence of SEQ ID NO: 3.

45. The method of claim 43, wherein the nucleic acid molecule encoding the PP protein has or comprises the nucleotide sequence of SEQ ID NO: 4.

46. The method of claim 43, wherein the mammal has a stem cell and the nucleic acid molecule encoding a PP protein, a fragment thereof, or a derivative of either of the foregoing is administered to the stem cell of the mammal.

47. The method of claim 40, wherein the tissue is in a mammal.

48. The method of claim 47, wherein the mammal is a human.

49. The method of claim 40, wherein the source of PP is administered in combination with another osteogenic factor or a growth factor.

50. The method of claim 49, wherein the other osteogenic factor or the growth factor is a BMP, LMP-3, a PDGF, an IGF, a VEGF, RunX, Osx, or an FGF.

51. The method of claim 40, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer.

52. A method of inducing periodontal regeneration in a tissue, which method comprises administering to the tissue a source of PP in an amount sufficient to induce periodontal regeneration in the tissue.

53. The method of claim 52, wherein the amount is sufficient to induce regeneration of the cementum, bone, periodontal ligament, or a combination thereof.

54. The method of claim 52, wherein the source of PP is a PP protein having or comprising the amino acid sequence of SEQ ID NO: 1, a fragment thereof, or a derivative of either of the foregoing.

55. The method of claim 54, wherein the fragment of a PP protein has or comprises the amino acid sequence of SEQ ID NO: 2.

56. The method of claim 52, wherein the source of PP is a nucleic acid molecule encoding a PP protein (SEQ ID NO: 1), a fragment thereof, or a derivative of either of the foregoing; wherein the nucleic acid molecule is optionally in the form of an expression vector.

57. The method of claim 56, wherein the nucleic acid molecule encoding the PP protein has or comprises the nucleotide sequence of SEQ ID NO: 3.

58. The method of claim 56, wherein the nucleic acid molecule encoding the PP protein has or comprises the nucleotide sequence of SEQ ID NO: 4.

59. The method of claim 56, wherein the mammal has a stem cell and the nucleic acid molecule encoding a PP protein, a fragment thereof, or a derivative of either of the foregoing is administered to the stem cell of the mammal.

60. The method of claim 52, wherein the tissue is in a mammal.

61. The method of claim 60, wherein the mammal is a human.

62. The method of claim 52, wherein the source of PP is administered in combination with another osteogenic factor or a growth factor.

63. The method of claim 62, wherein the other osteogenic factor or the growth factor is a BMP, LMP-3, a PDGF, an IGF, a VEGF, RunX, Osx, or an FGF.

64. The method of claim 52, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer.

65. The method of claim 52, wherein the method effectively treats periodontitis.

66. A method of inducing differentiation of a cell into a cementoblast, osteoblast, or periodontal ligament cell, which method comprises administering to the cell or a periodontal space a source of PP in an amount sufficient to induce differentiation of the cell into a cementoblast, osteoblast, or periodontal ligament cell.

67. The method of claim 66, wherein the source of PP is a PP protein having or comprising the amino acid sequence of SEQ ID NO: 1, a fragment thereof, or a derivative of either of the foregoing.

68. The method of claim 67, wherein the fragment of a PP protein has or comprises the amino acid sequence of SEQ ID NO: 2.

69. The method of claim 66, wherein the source of PP is a nucleic acid molecule encoding a PP protein (SEQ ID NO: 1), a fragment thereof, or a derivative of the foregoing, wherein the nucleic acid molecule is optionally in the form of an expression vector.

70. The method of claim 69, wherein the nucleic acid molecule encoding the PP protein has or comprises the nucleotide sequence of SEQ ID NO: 3.

71. The method of claim 69, wherein the nucleic acid molecule encoding the PP protein has or comprises the nucleotide sequence of SEQ ID NO: 4.

72. The method of claim 69, wherein the cell is a stem cell and the nucleic acid molecule encoding a PP protein, a fragment thereof, or a derivative of either of the foregoing is administered to the stem cell.

73. The method of claim 66, wherein the cell is in a mammal.

74. The method of claim 73, wherein the mammal is a human.

75. The method of claim 66, wherein the method effectively induces periodontal regeneration.

76. The method of claim 66, wherein the cell is a stem cell.

77. The method of claim 66, wherein the cell is a fibroblast cell.

78. The method of claim 66, wherein the source of PP is administered in combination with another osteogenic factor or a growth factor.

79. The method of claim 78, wherein the other osteogenic factor or the growth factor is a BMP, LMP-3, a PDGF, an IGF, a VEGF, RunX, Osx, or an FGF.

80. The method of claim 66, wherein the source of PP is formulated in a toothpaste, an oral rinse, a chewing gum, a dissolvable tablet, a dissolvable film, a gel, a natural biodegradable polymer, a synthetic biodegradable polymer, or a non-biodegradable polymer.

81. The method of claim 66, wherein the method effectively facilitates guided tissue regeneration.

82. The method of claim 66, wherein the method effectively treats periodontitis.

83. A composition comprising a source of PP and a carrier.

84. The composition of claim 83, wherein the composition is formulated into a toothpaste, an oral rinse, a chewing gum, a dissolvable tablet, a dissolvable film, a gel, a natural biodegradable polymer, a synthetic biodegradable polymer, or a non-biodegradable polymer.

85. The composition of claim 83, wherein the source of PP is a PP protein having the amino acid sequence of SEQ ID NO: 1, a fragment thereof, or a derivative of either of the foregoing.

86. The composition of claim 85, wherein the fragment of a PP protein has or comprises the amino acid sequence of SEQ ID NO: 2.

87. The composition of claim 83, wherein the source of PP is a nucleic acid molecule encoding a PP protein (SEQ ID NO: 1), a fragment thereof, or a derivative of either of the foregoing, wherein the nucleic acid molecule is optionally in the form of an expression vector.

88. The composition of claim 87, wherein the nucleic acid molecule encoding the PP protein has or comprises the nucleotide sequence of SEQ ID NO: 3.

89. The composition of claim 87, wherein the nucleic acid molecule encoding the PP protein has or comprises the nucleotide sequence of SEQ ID NO: 4.

90. The composition of claim 83, further comprising another osteogenic factor or a growth factor.

91. The composition of claim 90, wherein the other osteogenic factor is a BMP, LMP-3, a PDGF, an IGF, a VEGF, RunX, Osx, or an FGF.

92. The composition of claim 83, wherein the carrier is a biodegradable polymer, a biocompatible ceramic, or a combination thereof.

93. The composition of claim 92, wherein the biodegradable polymer is water soluble polymer or a non-water soluble polymer.

94. The composition of claim 93, wherein the water soluble polymer is polyethylene glycol, agarose, or alginate.

95. The composition of claim 93, wherein the non-water soluble polymer is polycaprolactone (PCL), polylactide (PLA), polyglycolic acid-lactic acid (PGLA), or a combination thereof.

96. The composition of claim 92, wherein the ceramic is selected from the group consisting of hydroxyapatite, substituted brushite, unsubstituted brushite, substituted tricalcium phosphate (TCP), unsubstituted TCP, amorphous calcium phosphate (ACP), or a combination thereof.

97. The composition of claim 83, wherein the composition is formulated into a paste, a gel, or a cream.

98. The composition of claim 97, wherein the gel has a molar calcium-phosphate ratio of about 10:3.


SEQUENCE LISTING (13 sequences)

SEQ ID NO: 1 - protein, 572 aa, Mus musculus
GIETEGPNKGNKSIITKESGKLSGSKDSNGHQGVELDKRNSPKQGESDKPQGTAEKSAAHSNLGHSRIGSSSNSDGHDSYEFDDESMQGDDPKSSDESNGSDESDTNSESANESGSRGDASYTSDESSDDDNDSDSHAGEDDSSDDSSGDGDSDSNGDGDSESEDKDESDSSDHDNSSDSESKSDSSDSSDDSSDSSDSSDSSDSSDSSDSSDSSDSSDSSDSNSSSDSSDSSGSSDSSDSSDTCDSSDSSDSSDSSDSSDSSDSSDSSDSSDSSDSSDSSSSSDSSDSSSCSDSSDSSDSSDSSDSSDSSDSSSSDSSSSSNSSDSSDSSDSSSSSDSSDSSDSSDSSDSSGSSDSSDSSASSDSSSSSDSSDSSSSSDSSDSSDSSDSSDSSESSDSSNSSDSSDSSDSSDSSDSSDSSDSSDSSDSSNSSDSSDSSDSSDSSDSSNSSDSSDSSDSSDSSDSSDSSDSSDSSDSSDSSDSSDSSDSSDSSDSSDSSDSSDSSDSSDSSDSSDSSNSSDSSDSDSKDSSSDSSDGDSKSGNGNSDSNSDSNSDSDSDSEGSDSNHSTSDD

SEQ ID NO: 2 - protein, 460 aa, Mus musculus
ESGSRGDASYTSDESSDDDNDSDSHAGEDDSSDDSSGDGDSDSNGDGDSESEDKDESDSSDHDNSSDSESKSDSSDSSDDSSDSSDSSDSSDSSDSSDSSDSSDSSDSSDSNSSSDSSDSSGSSDSSDSSDTCDSSDSSDSSDSSDSSDSSDSSDSSDSSDSSDSSDSSSSSDSSDSSSCSDSSDSSDSSDSSDSSDSSDSSSSDSSSSSNSSDSSDSSDSSSSSDSSDSSDSSDSSDSSGSSDSSDSSASSDSSSSSDSSDSSSSSDSSDSSDSSDSSDSSESSDSSNSSDSSDSSDSSDSSDSSDSSDSSDSSDSSNSSDSSDSSDSSDSSDSSNSSDSSDSSDSSDSSDSSDSSDSSDSSDSSDSSDSSDSSDSSDSSDSSDSSDSSDSSDSSDSSDSSDSSNSSDSSDSDSKDSSSDSSDGDSKSGNGNSDSNSDSNSDSDSDSEGSDSNHSTSDD

SEQ ID NO: 3 - DNA, 1719 bp, Mus musculus
ggaatagaaactgaaggtcccaacaaaggcaacaaaagtattattaccaaagaatctgggaaactcagtggaagtaaagatagcaatggacaccaaggagtggagctggacaaaaggaatagcccaaagcaaggggagtctgacaagcctcaaggcactgctgagaaatcagctgcccacagtaacctgggacacagcaggataggtagcagcagcaatagtgatgggcatgacagttacgagttcgatgacgagtccatgcaaggagatgatcccaagagcagcgacgaatctaacggaagtgacgaaagtgacactaactctgaaagcgccaatgagagtggcagccgtggagatgcttcttacacatctgatgaatcaagtgatgatgacaatgacagtgactcacatgcgggagaagacgatagcagtgatgactcatctggtgatggtgacagtgacagtaatggtgatggtgacagcgagagtgaggacaaggacgaatctgacagcagtgaccatgacaacagcagtgacagtgagagcaaatcagacagcagtgacagtagtgacgacagcagtgacagcagcgacagtagtgacagcagtgacagcagtgacagtagtgacagtagtgacagcagcgacagcagtgacagcagcgacagcaacagtagtagtgacagcagcgacagcagcggtagtagtgacagcagcgacagcagtgacacctgtgacagcagtgacagcagcgatagcagtgacagcagtgacagcagtgacagcagcgatagcagtgacagcagtgacagtagtgacagcagtgacagcagcgacagcagcagtagtagtgacagcagcgacagcagcagttgtagtgacagcagcgacagcagtgacagcagtgacagcagcgatagcagtgacagcagtgacagcagcagcagcgacagcagcagcagtagcaacagcagtgacagtagtgacagcagtgacagcagcagcagcagcgacagcagcgacagcagtgacagtagtgacagcagtgacagtagtggcagcagtgacagcagcgacagtagtgccagcagcgacagcagcagtagtagtgacagcagcgacagcagtagtagtagtgacagcagtgacagtagtgacagtagtgacagcagtgatagcagtgagagcagcgacagcagtaacagcagtgacagcagcgacagtagtgacagcagtgacagtagcgacagcagcgacagtagtgacagtagcgacagcagtgacagtagcaacagtagcgacagcagtgacagcagtgacagcagcgacagtagtgacagcagcaacagtagtgacagcagtgacagtagcgacagtagtgacagcagtgacagcagtgacagcagcgacagtagtgacagcagtgacagtagtgacagcagcgacagtagtgacagcagtgacagcagtgacagcagtgacagcagcgacagcagcgacagcagtgacagcagcgacagcagcgacagcagtgacagcagcgacagcagcaacagcagtgacagcagtgacagtgacagcaaggatagcagttctgacagcagtgatggtgacagcaagtctggtaatggcaacagtgacagcaacagtgacagcaacagtgacagtgacagtgacagtgaaggcagtgacagtaaccactcaaccagtgatgattag

SEQ ID NO: 4 - DNA, 1383 bp, Mus musculus
gagagtggcagccgtggagatgcttcttacacatctgatgaatcaagtgatgatgacaatgacagtgactcacatgcgggagaagacgatagcagtgatgactcatctggtgatggtgacagtgacagtaatggtgatggtgacagcgagagtgaggacaaggacgaatctgacagcagtgaccatgacaacagcagtgacagtgagagcaaatcagacagcagtgacagtagtgacgacagcagtgacagcagcgacagtagtgacagcagtgacagcagtgacagtagtgacagtagtgacagcagcgacagcagtgacagcagcgacagcaacagtagtagtgacagcagcgacagcagcggtagtagtgacagcagcgacagcagtgacacctgtgacagcagtgacagcagcgatagcagtgacagcagtgacagcagtgacagcagcgatagcagtgacagcagtgacagtagtgacagcagtgacagcagcgacagcagcagtagtagtgacagcagcgacagcagcagttgtagtgacagcagcgacagcagtgacagcagtgacagcagcgatagcagtgacagcagtgacagcagcagcagcgacagcagcagcagtagcaacagcagtgacagtagtgacagcagtgacagcagcagcagcagcgacagcagcgacagcagtgacagtagtgacagcagtgacagtagtggcagcagtgacagcagcgacagtagtgccagcagcgacagcagcagtagtagtgacagcagcgacagcagtagtagtagtgacagcagtgacagtagtgacagtagtgacagcagtgatagcagtgagagcagcgacagcagtaacagcagtgacagcagcgacagtagtgacagcagtgacagtagcgacagcagcgacagtagtgacagtagcgacagcagtgacagtagcaacagtagcgacagcagtgacagcagtgacagcagcgacagtagtgacagcagcaacagtagtgacagcagtgacagtagcgacagtagtgacagcagtgacagcagtgacagcagcgacagtagtgacagcagtgacagtagtgacagcagcgacagtagtgacagcagtgacagcagtgacagcagtgacagcagcgacagcagcgacagcagtgacagcagcgacagcagcgacagcagtgacagcagcgacagcagcaacagcagtgacagcagtgacagtgacagcaaggatagcagttctgacagcagtgatggtgacagcaagtctggtaatggcaacagtgacagcaacagtgacagcaacagtgacagtgacagtgacagtgaaggcagtgacagtaaccactcaaccagtgatgattag

SEQ ID NO: 5 - protein, 936 aa, Mus musculus
MKMKIIIYICIWATAWAIPVPQLVPLERDIVENSVAVPLLTHPGTAAQNELSINSTTSNSNDSPDGSEIGEQVLSEDGYKRDGNGSESIHVGGKDFPTQPILVNEQGNTAEEHNDIETYGHDGVHARGENSTANGIRSQVGIVENAEEAESSVHGQAGQNTKSGGASDVSQNGDATLVQENEPPEASIKNSTNHEAGIHGSGVATHETTPQREGLGSENQGTEVTPSIGEDAGLDDTDGSPSGNGVEEDEDTGSGDGEGAEAGDGRESHDGTKGQGGQSHGGNTDHRGQSSVSTEDDDSKEQEGFPNGHNGDNSSEENGVEEGDSTQATQDKEKLSPKDTRDAEGGIISQSEACPSGKSQGIETEGPNKGNKSIITKESGKLSGSKDSNGHQGVELDKRNSPKQGESDKPQGTAEKSAAHSNLGHSRIGSSSNSDGHDSYEFDDESMQGDDPKSSDESNGSDESDTNSESANESGSRGDASYTSDESSDDDNDSDSHAGEDDSSDDSSGDGDSDSNGDGDSESEDKDESDSSDHDNSSDSESKSDSSDSSDDSSDSSDSSDSSDSSDSSDSSDSSDSSDSSDSNSSSDSSDSSGSSDSSDSSDTCDSSDSSDSSDSSDSSDSSDSSDSSDSSDSSDSSDSSSSSDSSDSSSCSDSSDSSDSSDSSDSSDSSDSSSSDSSSSSNSSDSSDSSDSSSSSDSSDSSDSSDSSDSSGSSDSSDSSASSDSSSSSDSSDSSSSSDSSDSSDSSDSSDSSESSDSSNSSDSSDSSDSSDSSDSSDSSDSSDSSDSSNSSDSSDSSDSSDSSDSSNSSDSSDSSDSSDSSDSSDSSDSSDSSDSSDSSDSSDSSDSSDSSDSSDSSDSSDSSDSSDSSDSSDSSNSSDSSDSDSKDSSSDSSDGDSKSGNGNSDSNSDSNSDSDSDSEGSDSNHSTSDDTSDD

SEQ ID NO: 6 - DNA, 17277 bp, Mus musculus
gaattcttttcccattggtaacgtaaaagaccactacttaattgagttagcttaggctcaacaaacagactttatacaacttaacttccttcacatttatgaaaaattaatcagtatcggcactgagaaggcagaaacaggtagaactccatgagtttcaggccagcctgatctacataggaattctaggacaagcagggctaggtagagataccctatctcaaaaaaccaaaacccaaaaacattacgtttaagcagatttagttttgaccctaaatgtttgtcttagtgaaggtcccaaatgctcttagcaaatgtttctttgtgtagttggagagtgttgtgtgctaatacagctatcaagcacttctgtttagacaccgaagatcttcttaactctccatcaggtctggagagctgttcaaatctgctattacaaccaagttaggaagaggaaggcaattcctgaggaaagtggcattcttaaatatgattggccctttaagatgctcaaagaaccaagaaccatgcagtgtaaataatagcaaagtgtttactatggaagtgcagcttcgaggaaactcccttcctatcactggaacctgtccaatccctacctacatgaatatgttgtttaattctctcagtataaagctctgaagatgctgttgctggatagtgatttaatatttctgatcatatgtgtttgacatctttcagtagtgtgacataaaaacatggacacatccctaagctggtacacagagactccaattgcctagtgtggagctcataagctagagaaatggctcagggatcatcttgtatatccagggctcgagagaatgatgggttcaggcaagtactttttcctttctggaagcacagcctgttttcctattctgtactctatagtttacacatatagtggagcaaagaatgaaagctgtgtctgtggtgtgtgtgtgtgtgcactctgtacttacgcatagataccttacaccatgtttcacctttggaacagctatttttaaatttagtttgtattaaattaatagattataaagaaaaacccaaaacctttatgtcagtgtttagattaaatcagaaaggtttcctgaagttactgtttataaattcttttaaagatcccttaggcagtgtcaagactgttgcatgcggacagccgcttgaattatagcgcaccaactttaatatgtacctcaggaatgataggggtcttaaatagccagtcgtatttactagagaaacctagagttttcttagattgccgacctaagcaagaggagaaatgcagggtgacagagtctaagtggctcttttcagatatatcacactgattatctatatttaagacacaaaacagtcttccaggagctatttaattaagtgaaagtaagtctagtccttttggaaccaaaggtctcagtgagccaacgtaccggcgagcgagggagtggggcgttattacagcctcataggcacactgactctttaaacccccacatcagggatcctaagcagtgattggttgagaaaattatcaaactgaatttaaatttcagcaggtacaaaattgtcacgcaaaaagcccaggacagtgtgccactctcagcctggaaagagagataaggaaatctggattttcaaagtcccctcggaggctttgaaggtaagatggactccctcctgccaggagccaactgtctcctgttgagagaatctccagctgcagagatgagggtgacttgggataaagtttttaactcttcaggtctacactatatattaaagataatgtgtgattcaggaaggggtgctaagccatctgatgagaccatctgataagacgacgaatcactggggagcagaactgattttgccccagtatattgttgagactttatctcctataggaaaaacctaagatgaaacaaacattctaattgtattaattaaaaaaaaacagtacctgaagggttttatgtatagttctctatagctctatttttgttattttcattcaggaaaatacttttaagagctataaacctagtcaaaggtgttttacagccttgtccttggaatgttgggagtgttgggatttaacaaatgagaatcacacactgtcttcctcttcgagacagagacatggatgatgcagtgtccaaacaccagctcttcctgagagataagctgggtttgggggtttgatttaatcatggctcttcatgatttcaaggtctgcctagtgtttatgattaaagctctatggcgaaaagaattgtggttcctcccagggctcagtatctgcctgatattaatcttccgatgttcactgactggacctaataaataaatctccatttaaacttagtatcttgactcagagtcaacttaggatctgggagcgtaattttctggcatgtgatgtgaagtttctaaaagtagacgctcaaacagttttatgtagaaaacacacagatctgtcaagctgatttttcagctccaaatttcatgataataggtttagggaaaacaaagacatattgcctcaagttggcaaaaattgaggtggaaatttgaatgtggtcactttgaatggttttgatttaagaaaaaatagataacttgtattgtaaatatctttaaaatatttttattcattccctgagaaatttgtgtggtatgttctgattgctctccccagatctgcctttgttctttactcacacaactttgtgctctttttgtaaagaaacaaaacaagagccatgcacaccagtttgtgctcctcaaatgtactcagctgtgtggccatctgctgggttctggttgccttaccaggggctacattcttggagaacactgcctttccttttttcccaccacctattgttaattgttcttcatgtccagctttcctctccttgctgggatttggtctgacttgggcttgcacggtcgggtgcaggctgtcagaagcgctgtgaagatagctcgggtagtttaagtctacctcaggcattccaacaaggccctcacaatgaggctttgcgtttcctggtcttcttagtgagtgatatattcattctaactggctattcatacatttcatctagtgtggggcaataaatgggacaatttaaaggagcctcaattctaatgactggttatttccaccagggtctttgatatggttgacctgccttgccaacaggtgcaagtatcatatatgtcagtgctggagtggaaatgtggtgtgtgtgtgtgtgtgtgtccgtgtgtgtgtgtgtgtgtgtgtgtgtgtaaggagggatggaaggtggatggtgggagacaggaattctcagatggtcagatttcagtttagaaattatatgtgtgtgtgtgtgtgtctgtctgtctgtctggactttattgcaggtacctttccaggaccaggtatccccagttcacactcggtttagagttgccaagctcaagtataagcttggcttggtagacagatggccttcacctcaactcctggccctggggctttgtctcaaggcacctcattttagtttgtagaataattgaagggaccccagcttttcttagctttctcttgacagctataaggaagggtgaagcatctttttcagagatcctagaattgtgttctcacttctgtcaagtaataaacaatatatattcattgatgttttattctattcccctattaaccttggattttaatcaaggacattttatgatgtgcaaggtggtaatcattaattcttgtggaaggtcacaagataggagaaaacaattctttctatagtaaaacaccatgatacaaataaatttagttttagaaaatgggaacctgaagttttgattcacatagatttttatagttttacaggctccattccaatgtatgaaaaatatgtatctgattctgtgaatttgcattgcaaagggtgaaagatttcactcttgaagcctctctccttcagctcctccctcagtccgagactgcatagtgcccgggtaagggtggggtgtcctttgtcctcaggagtgcttgttcagcagcaggctctgcaaggtgacctttgctttgctcagaagacactgatgatcaagatgctggcgtgggctccgagacctgatgccagtgaggaggaagatggggtagctaggcaacttcaaaacagtgcaatgtgctgccagcatcgagcgagcggagggtgcacaagctgatgctgtgtgaggaagggagctaaagatgccttcagaaagctttttgggggtgattcttctgccaacccctaggatattgtgagctacagagttattaaaccagactgaggaaacaaaagcccaataaagctattgaaagtgcccaagctcagagagcagatagcaggggaaggatttgaattcagggatctgaaaccaaatcctgtgttctctctcctagcctaaactctctcttccttaaacactgtaagaggaagatttcttcctcttactgggataacgcccaattctatatagaccaggtgggaaattacaagtgctttatcatttacaatctacttttagttaatgatgcttaaagctagcccaggagagacgttaccctcatggataacagcatagggccagagccacgagctatgtactctgtatcttcatggctgttgcttccacaggcaggtagagtcagaagccatgacagtcctgagcatgcagaggcccccacatacccaggtttatttctggaacctggggtgttttctcacattagtactttctccttgtcctagaaaagggccaaatgtaagaccaaaatattggggtactgtggctgtcatctttcatcttatgacccgttttgtggtgttctttgttctaaacagacattgattactactcataatgaaaatgaagataattatatatatatgcatttgggcaactgcctgggccattccggtaaggcttttcccaatcaagcttcttactttgctgtatctttcaacccaatgttgaaatgtaacatatttccttatggttttacagagaagttgagtctaaacattaatagaaatgttaagatttgcattgcagctattatgtgatatcatatggggtctcgatgaaggcaaacacatgcaccaatgcatgctccctccattcctgttgaaacatcctaatgaaagaatgaccctttttttttaaagtttatccaaattaattcagtgctccaaagtcatgaagcttgtctgcttcattccacacgaattccactgtaatgtcaacacactgtattctgtttgggaaaaaactgaagaaagaacaggagctaaaagtcagatctttcaatgtttcatgtgtgcatttgtgtgttcactgtgggaaatctggagcatcagaacaagtacaaaggcagaaacattaagaaagtcgatctgtttgtcatttcatcagctggcttccacatctaacattgtcacagggcgtcacataaccagattctgggttgttcctgtacttgagaagttttgtaagcactccgagctcactcttgcagggtgagaattatcagctaccggggctgcttctccagtggtccactctcatgttgctttaggggtttggggctgatcgacaacaacattataaaaatcctcactttctctgcctgaaaccccacataagcaccgcagcaggctccttctcttctctacacgatcagagtgcgatctgaccttcatataatatctgtgtctcaacctctgcaggttccccagttagtaccactggaaagagacattgttgaaaactctgtggctgtgcctcttctaacacatccaggaactgcagcacaggtaaaagacagaaatacgaatgtcctttctttttctgttttcaaggcccttttacactttaccactttctctaaaatatccacccttttttttcagttggccttatttgaaaatgatagccacaactgactttcaattgtgtctccttttcagaatgagttatctatcaacagcaccactagcaacagcaacgactccccagatggcagtgagataggagagcaggtacttagcgaggatggttacaaaagagatgggaatggctccgagtcaatacatgtaggagggaaggattttcctactcagcccattttagtaaacgaacaggggaacactgctgaagaacacaatgacatagaaacatacggtcatgatggggtacatgcgagaggagagaacagcacagcaaatggcatcaggagccaggtaggcatcgttgaaaatgcggaggaagcagagagcagtgtccacggacaggctggtcagaatacaaaatctggaggtgctagtgatgtaagccagaatggagatgcgacccttgtccaggaaaatgagcctccagaagctagcatcaagaatagcaccaaccatgaggctggaatacacgggagtggggttgctacacatgaaacgacgcctcagagagaagggctggggagtgagaaccaggggactgaggtgacaccaagcatcggggaagatgcaggtttggatgatactgatgggagtcctagcgggaacggggtagaggaggatgaagatacaggctctggtgatggtgagggtgcagaagcaggagatggaagggagagccatgatggcactaagggccaggggggccaatctcatgggggaaacactgaccacagaggtcagagttcagttagtactgaagatgatgattctaaagaacaagaaggcttcccaaatggacacaatggagacaacagcagtgaggaaaacggtgttgaagaaggcgacagtacccaggcaacgcaggacaaggaaaagctcagccccaaagacacccgagatgcagagggtgggatcatcagccagtcagaagcatgtccttctgggaagagccaagatcaggtaagtttagagggcggcgacttccattcttccctccatactgtgatggctgtaccaaataactccagacaaacacgagagataaaaccccaaccaagcataaaagtactatgctaagcatctgggttctattttagttacattgagtattctaatgaaaaggctggaattcttatagactttcatgtaggacaatttaaaaatatatatttattttattttatgtatagatgagtatactgtagctgtcttaagacacaccaaaagaaggcatcagatcccattctagatgactgtgagatactatgtgattgctgggaattgaactcagggcctctggaagaacagtcagtgctcttaacccctgagccacctctccaatatgtctctgatataggacaatttttaaaaattcacaaacttctgtaaaattagtcagaatgctagaagtcaagctgcataacggttccatgatgtctttgtaagacattttattagtttacattcatcacacagaatgaccagcttcactatgacactttcattattatgcttcaagcccttatgagttagaaacctggatggcttattagaggatccaaaccctgatacagagcacatttgcattcaagtactagatcagcaggcgtgcatgaatcactgcactgacagcctatactcctgttcctaaggtcacttcctgagacagttctcctcagaccatgatgttttgtagcaaatattcactaattatccattcttctttatatcgttccacagggaatagaaactgaaggtcccaacaaaggcaacaaaagtattattaccaaagaatctgggaaactcagtggaagtaaagatagcaatggacaccaaggagtggagctggacaaaaggaatagcccaaagcaaggggagtctgacaagcctcaaggcactgctgagaaatcagctgcccacagtaacctgggacacagcaggataggtagcagcagcaatagtgatgggcatgacagttacgagttcgatgacgagtccatgcaaggagatgatcccaagagcagcgacgaatctaacggaagtgacgaaagtgacactaactctgaaagcgccaatgagagtggcagccgtggagatgcttcttacacatctgatgaatcaagtgatgatgacaatgacagtgactcacatgcgggagaagacgatagcagtgatgactcatctggtgatggtgacagtgacagtaatggtgatggtgacagcgagagtgaggacaaggacgaatctgacagcagtgaccatgacaacagcagtgacagtgagagcaaatcagacagcagtgacagtagtgacgacagcagtgacagcagcgacagtagtgacagcagtgacagcagtgacagtagtgacagtagtgacagcagcgacagcagtgacagcagcgacagcaacagtagtagtgacagcagcgacagcagcggtagtagtgacagcagcgacagcagtgacacctgtgacagcagtgacagcagcgatagcagtgacagcagtgacagcagtgacagcagcgatagcagtgacagcagtgacagtagtgacagcagtgacagcagcgacagcagcagtagtagtgacagcagcgacagcagcagttgtagtgacagcagcgacagcagtgacagcagtgacagcagcgatagcagtgacagcagtgacagcagcagcagcgacagcagcagcagtagcaacagcagtgacagtagtgacagcagtgacagcagcagcagcagcgacagcagcgacagcagtgacagtagtgacagcagtgacagtagtggcagcagtgacagcagcgacagtagtgccagcagcgacagcagcagtagtagtgacagcagcgacagcagtagtagtagtgacagcagtgacagtagtgacagtagtgacagcagtgatagcagtgagagcagcgacagcagtaacagcagtgacagcagcgacagtagtgacagcagtgacagtagcgacagcagcgacagtagtgacagtagcgacagcagtgacagtagcaacagtagcgacagcagtgacagcagtgacagcagcgacagtagtgacagcagcaacagtagtgacagcagtgacagtagcgacagtagtgacagcagtgacagcagtgacagcagcgacagtagtgacagcagtgacagtagtgacagcagcgacagtagtgacagcagtgacagcagtgacagcagtgacagcagcgacagcagcgacagcagtgacagcagcgacagcagcgacagcagtgacagcagcgacagcagcaacagcagtgacagcagtgacagtgacagcaaggatagcagttctgacagcagtgatggtgacagcaagtctggtaatggcaacagtgacagcaacagtgacagcaacagtgacagtgacagtgacagtgaaggcagtgacagtaaccactcaaccagtgatgattagatcagagagaacccatgatatcctctgtgtgacctcttggtgaggtgatgggaaggcagtgaaggttcctaacccaatgatgacaggagagatgtgcagactgtgtggaacccatggagctcatagggagtggagccgagctccagctctctcagagagaatctgggtgtaccacctttggtacatgtgtgttaaaatatattcatgttcagaaaatatttttaaaaggataaatctaaacaatactttaacaggaactgaagaaatcactaagacacatagcttcgatttgaatggcgggtgctttaaagagcagagctagcaatgtcacagcctgctgcagcctcctccctcagtgctccgggcaccagagagctagtcttcatgttgtgcagtgagtaatgctgttctgtgacattcaactcaactactctgtcatttatttattccggggaaaattacatttagggcataatcaaaacaccgctgcaactactggccctatccaaggtgctgagataatctttgtgatgagacaatagctatacattatgaaaattccgaagaatgaatgagaaaagagccccaaggatggcttgggcaggatctgacacatgcggttaaatttctgcatgggatggatatgtactaagtccccaacccctgcactttgaacagtgtctcccttccagcagtggccctcaaaccttaaataaacgagcaacacggatggatgatttcgggaggtgggatcatattctgagctctccatgtaccactgtgttattagttttcttcgaatcacagctcaaacagtttaatcaagagttgtaaggctgtgcgtgacaagagtgggaccctgtttgggctctagggctcctctgaaagcaagagaggtaatgagaataaaccacaccaagacaggaggtgtgaactgggattgtctcaagaaaaccttaaccctcaagccttaaggatatttttgaagatttagggttttcctttgtcatttccctatttccccacataggcagttatgccaaatttgggttaaatagaaactattaaatacattataatgataatctactctattctcattttaggcttattttacccagagtttcagaagagtttcttttctcaggtgctcacctccttttgtgagagtttctgagttaaggaatattgctgaggctttcacacgctgctatctgtaaacgcgttgtaacgcccacactgtaaagctccaggcttctgtgagctgccacagctgtgacgtgactccagacccctcaccagaaagtaaaggttcagtctttgccttctactagaccccaaactctcctttgtttgctgtaacttatgaagcacctgcctctagtaacccgccacacccactcatcgaggttgtgatcactaaagccatgggtagaaaactcatcgtaaactgtgtaagaaatgtaaaggaagagataatgaacttcagtattataataaacatctatttatacaattgctcactgagtaaattcttcattcatagtctgcaaacattgtcccctcccccattgtaaaatctggtgtgtaagattatacttcttacacatatttagccattcttattaaaataggtatttgtgaacacaaaatacaaacttcaaatactacttaaaaacagtacacataatactaaacctttgtcatccaacccacaatttctttttcctagaggcaattcctcttactaatgttttacagatattccagaaatattgtatgactatgttcacctttaagaagtctgtggtattgtaccacacacaatgcactcattttacatgtcaacttagcagtatgccttgaacattggctcatagcacgtagatcaacttcatttctttgtagttctgctcatttcatgaaccagtataagatatttatcctgtgctcatgatatctagataatagccccaagtaagtgtcatggtcactggtttatttctgtgaagagacaccatgaccacagaaactcttataaaggaaagcatttaattggggcttgtttacaggttcagaggtttaagtccattattgacacagtggggagcatggtagctgaaagttctacatctgaatccgtaggcagaggagaaggagctactgtgttggggttgatcgtgtgctgctgtgtattcaaatactggcccctgagatctgattgccccatgagatcctcacatacaccaagtgatgcaatctaaaccttgcttcccaagaattggtcaataaaagactaaagtctgaaattgggcagtagagagaaaaaggtgggagacttgaggatcaaatagagtgaggggtctcaggagagaccaaagatggaggagagaaggaggcgacaaagaaaggaggtagctgccataataggagatggatcatgagcacgtggacaggagcaactgacaagggacatatggtctggatgtaagttacaatagctcaaaaactacccaatataggcttacagcttgtaaataaaataccaggaccgtgtgtcttatatgggctagctggaatatataattccttttcaaattggcgcccacatgggacaataagagcccaagcttacagcctgagaagggtaggggtggggtggggtgatgaggtgggagggtggggtgaggtgggatggggatagtcagactaactggacaagaggcatggtctcttttaaaaaagaacgaaagcagacaaaagcctcagatacactagaaaaaactaggcctggagctatgggtgaaggcctgaaacaacgcagaagcatggaagattggggaggcctgatcaggactccggttgagcgggcaagctggttgccatagacacgtgctggccccaaggagtctttagacacacagcagtttataatagagtacttctccctaactgcaataagacttaaaaggccccaacttctgaactggtaaggtcttaagtttaaaattggtaaattgatatctttaaggaaagagtcagagataaaatggaaaaatactttccatgttaaaaaaaaaaaaaaggaaaacaggacagcagaaggcccttggattcttgtatcatttcattttagttgtcatggagctagttacaatacgttcactaatgatcacaattttatgtcctctctctaagaatgttcaaaataaaacagacttacataaggagagaactgagaggtggggtggtgattacaagcaatatagatagagaaaagaaaaaaaagggcccttttccggataagaaaaaaaaggaccattgggcggggcaagtttggaactcagagctctctggctgtgagatgcttgtctgctctttctgctaagggctcactgatacaatgttgcaacaccttaattccgaggagtaacatacaaggttttgctgctacatatagagtcaataaattttattattttattggctacaaaatctttaaaacttttcatgctattatcttgaatggcatagataaaaatttatatcgaagcttggttacagtccaaaactagtttaagaaagatagttgtctttcacctgctcaaacaatcaacaaaaatcttcattgactgacctgtgcaccttgcatagcccatacattgttggtacagaactgtatattacttgtgagaacttacttgttcacttaaaataacaaccaaagaagcagccccaacaagatatagccttggggatgctgggatgcctgctcctgcctcagattgccttgatgatgtttccttgggagacttgtttccagaatagcttcagggagggctgctgaccccagatgacctcagtttgttcagtcttgcagatgggtccagcaagggagtcaattaagccctgcaatttcctatcccacagagactggacagcaaatgatacagttatttctcccaggatttggccagtatcctaattttcttaggctctccaagagatgtcatcaaccctaaacagcagaaagcaatttaaagagaacatgtcaccccattccaaagagatagggtatatgatttttagttattctattgggtgatggatgcttgttgttataaaggggttggttgcaagtttttaaatggtcttgatcagggaaaaaaccaaggtatagcaggttagactcaaggatttcccttttttctttcctctatccttctttcttatatagggaaagaagggttcaaaacaaacagggagatacaggaaaatatagaaataataagtagattattaaatctactcttagagctactactagccaaaaatcttacattcttatagatcttcgtatattgatacaaaattgaggttatattttgttatattgctatagatctttatatattgatacaagatttgaagtactcatattggcattggacagatgtaactcatttgaagattttgtgtaaagttctagtctcttctaaagctggtattacaaactctttaggataattaagaaatacaagttgatagacagtcaaacacatggtaatattagatactagaatagtttattacagtaaaatacttcctagctaaaaccaagtttacctattcagatattctgattagatagatgatcttcaaaatccttggagacctacagaatatgacattttaaggttttttttaaattaaattaagacttttcttgacattgagacatgtcagctcctcgcagtaccccattcaacttggaaaaatatgatgagcattggaggaccttcatttgaagatggattctgctggagtccaactctgagtgaggaccagggctctcatgctcattaatgctacttaagtaataggttctatggaagactcaatttctgcatagctgactctcccagggaactaccatgaattttattcttaataacaccagattttgtaagaattgttacattatcgcagccccagccttccatgaggggcccttagaagcaagaaattcaaatattaatcagaaacacaagcatacgttgtgtagcaaatttccaccaagagcagcaatgggtcagttctggttgtcccagcactggaacattgtcaagcaatgcctgcaagagcttggcatgaccaggctttcattatggcaagctagtcactgggcaaagagaatgttctaacttcatttgcagacagaatgctcttcaaaatggagaaaatttggatgcaggcaaagtcgactgccaagccctgccaagacagggtaagaatatccttcatagttcctgctccacaaacatgcctgtcagatatactggggcagaggcctgaagacagatgttccagtgttatagagaattttggggattctccagtcagctagatgcttgccaattctatagttttggaagctgcttgcctacacttcctacaaactcagttaattatcccttcccaagtctctgatggggttgaagattatatagtcatagtctcacaatgaaacataacaaagaatctaagaaagtgctttagggtctaaggaggtgttttaaggttggtaaatgaagatcataggattagatggtgttttatgaaggttggaggaaattgtaaatgggtgttttaggttggtaaatgcaaattatgaaagttagaggatttaaatgcttaagatggtaatggaaaaagtaatttaaatacagaactctgaactcaccaagattcaatagataaaaaatatcttctcctaagttgccaaatacagatggactggacattgtgaatatatttattacccatggatttcataattgctcttactgatatagttccttattgtaagagaaagatccttttttatttagacaaaaaaggggaaatgttggggttggtctggtgctgctgtgtactcaaatactaaatactgggtcccaagatctgattgctctcaatgagcagcagatctttacacaccaagtgatgccatgtaaaccttgctccccaagttattggtcgataaaaggctaaagtctgggattgggcagtagagagagaaaggtggaagacttgaggatcaaatgagggtgtctcaggagagatcaggggaggagataagaaggaagtgacaaagagaggaggagggtgccatgagaggagatggatcatgagcacatggccaggagaaacagcaactgacaagggacatatggctgggatataagttacaatagctcaaaaagttgcccaatataggcttacagcttataaataaaataccagaatcatgcatctttaatgtggcttagctagaatatgtaattccttttataccactgggcttagaatgtcacccccagtgacacacttcctccaaaaggccacatatcctaatccttctcaagtagtgccactttctgatgactaagtattaatgtattggggccattcttatccaaactaccacagtcataatacatctagcaggttcttagaaagctttctccctaaagagtatttttatgaggttagatgctttaggacctagcattatactggaactcatgaaggaagattatgaccttgtttttcttgtataaccatttatatctgaatttggaatttcagggcaaaaatggaggagacacaattaaaaatgtctcaaggttcaatcctttgaatgccagaaaagtattattagggaaaaccttacgttatttaccagaataaagattaataagcaatttcctcatactgttcatcagggcaatggtgtttaggttctatttctaatgacatgtctctttgttagggaattcccatgagcactcaggtgttcatggagaccagaagaggatgtcagatctcctggagctggagtgaagccacttgtaagctgcctgatgtggatgctggaaatcaaacttgaaacctttattagcccttatactcttaattgctgagtcatctctccagtttctgacagcagtgttccctaaatcccaggttgctaatcaactagtcacttattataattatatcaatttaatgagttacaaaaatacttaagatgaaagagtaaggtaaaatcataacagtgtgttgtgaaactatatacatatacatattgtcttagttaggatttactgtgggaacagacaccatgaccaatacaagtcttataaagggtaacatttaattgagatagcttacaggttcagaggttcagtccattatcatcaaggcatggcagcatccaggtaggcatggtgcaagaggactgagagttctacatcttcacctgaaggttgctagaagaatactgacttccaggtagctaggatgagggtcttaaagcctacacccacatttacacacctactccaacaagactataccaactccaacagggtcacaccctctaatagtgccactcccttgggctgagcatatgcaaaccatcacacacagatatgttgaagtgcgcctatgctagagatgcatgcaatgtctttttaactgttggttgtggttaggaaaattagagaaccattggtttaggaagacattactgccctggtaatttgatactgattttcaacattcacctttctccttacaaacctctaacttgcttgcccaactttgaagatggaaaatttaaaagaaagcacaagaaatattgggggtgtatctgaatgggtagaagggatcgaaatgggtagaagggatcgaaatgggtagaagggatcga

SEQ ID NO: 7 - protein, 1253 aa, Homo sapiens
MKIITYFCIWAVAWAIPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSSSSDSSNSSDSSDSSDSSNSSESSDSSDSSDSDSSDSSDSSNSNSSDSDSSNSSDSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDSSDSDSSNRSDSSNSSDSSDSSDSSNSSDSSDSSDSSDSNESSNSSDSSDSSNSSDSDSSDSSNSSDSSDSSNSSDSSESSNSSDNSNSSDSSNSSDSSDSSDSSNSSDSSNSGDSSNSSDSSDSNSSDSSDSSNSSDSSDSSDSSDSSDSSDSSNSSDSSDSSDSSDSSNSSDSSNSSDSSDSSDSSDSSDSSDSSNSSDSSDSSDSSDSSDSSGSSDSSDSSDSSDSSDSSDSSDSSDSSDSSESSDSSDSSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 8 - DNA, 4221 bp, Homo sapiens
atgcaaaagtccaggacagtgggccactttcagtcttcaaagagaaagataagaaattctggattttcaaaatccttttgaagccttttaagccattgattattattattcctaaagaaaatgaagataattacatatttttgcatttgggcagtagcatgggccattccagttcctcaaagcaaaccactggagagacatgtcgaaaaatccatgaatttgcatctcctagcaagatcaaatgtgtcagtacaggatgagttaaatgccagtggaaccatcaaagaaagtggtgtcctggtgcatgaaggtgatagaggaaggcaagagaatacccaagatggtcacaagggagaagggaatggctctaagtgggcagaagtaggagggaagagtttttctacatattccacattagcaaacgaagaggggaatattgagggctggaatggggacacaggaaaagcagaaacatatggtcatgatggaatacatgggaaagaagaaaacatcacagcaaatggcatccagggacaagtaagcatcattgacaatgctggagccacaaacagaagcaacactaatggaaatactgataagaatacccaaaatggggatgttggcgatgcaggtcacaatgaggatgtcgctgttgtccaagaagatggacctcaagtagctggaagcaataacagtacagacaatgaggatgaaataattgagaattcctgtagaaacgagggtaatacaagtgaaataacacctcagatcaacagcaagagaaatgggactaaggaagctgaggtaacaccaggcactggagaagatgctggcctggataattccgatgggagtcctagtgggaatggagcagatgaggatgaagacgagggttctggtgatgatgaagatgaagaagcagggaatggaaaagacagtagtaataacagcaagggccaggagggccaggaccatgggaaagaagatgatcatgatagtagcataggtcaaaattcggatagtaaagaatattatgaccctgaaggcaaagaagatccccataatgaagttgatggagacaagacctccaagagtgaggagaattctgctggtattccagaagacaatggcagccaaagaatagaggacacccagaagctcaaccatagagaaagcaaacgcgtagaaaatagaatcaccaaagaatcagagacacatgctgttgggaagagccaagataagggaatagaaatcaagggtcccagcagtggcaacagaaatattaccaaagaagttgggaaaggcaacgaaggtaaagaggataaaggacaacatggaatgatcttgggcaaaggcaatgtcaagacacaaggagaggttgtcaacatagaaggacctggccaaaaatcagaaccaggaaataaagttggacacagcaatacaggtagtgacagcaatagtgatggatatgacagttatgattttgatgataagtccatgcaaggagatgatcccaatagcagtgatgaatctaatggcaatgatgatgctaattcagaaagtgacaataacagcagtagccgaggagatgcttcttataactctgatgaatcaaaagataatggcaatggcagtgactcaaaaggagcagaagatgatgacagtgatagcacatcagacactaataatagtgacagtaatggcaatggtaacaatgggaatgatgacaatgacaaatcagacagtggcaaaggtaaatcagatagcagtgacagtgatagtagtgatagcagcaatagcagtgatagtagtgacagcagtgacagtgacagcagtgatagcaacagtagcagtgatagtgacagcagtgacagtgacagcagtgatagcagtgacagtgatagtagtgatagcagcaatagcagtgacagtagtgacagcagtgatagcagtgacagtagtgatagtagtgacagcagtgacagcaagtcagacagcagcaaatcagagagcgacagcagtgatagtgacagtaagtcagacagcagtgacagcaacagcagtgacagtagtgacaacagtgatagcagcgacagcagcaatagcagtaacagcagtgatagtagtgacagcagtgatagcagtgacagcagcagtagcagtgacagcagcagtagcagtgacagcagcaacagcagtgatagtagtgacagtagtgacagcagcaatagcagtgagagcagtgatagtagtgacagcagtgatagtgacagcagtgatagtagtgacagcagtaatagtaacagcagcgatagtgacagcagcaacagcagcgatagcagtgacagcagtgatagcagtgacagcagcaacagcagtgacagtagcgatagcagtgacagcagcaacagcagtgacagcagtgatagcagtgacagcagtgatagtagtgacagcagcaacagcagtgatagcaacgacagcagcaatagcagtgacagcagtgatagcagcaacagcagtgatagcagcaacagcagtgatagcagtgatagcagtgacagcagtgatagcgacagcagcaatagcagtgacagcagtaatagtagtgacagcagcgatagcagcaacagcagtgatagcagcgacagcagcgatagcagtgacagcagtgatagcgacagcagcaatagaagtgacagtagtaatagtagtgacagcagcgatagcagtgacagcagcaacagcagtgacagcagtgatagtagtgacagcagtgacagcaacgaaagcagcaatagcagtgacagcagtgatagcagcaacagcagtgatagtgacagcagtgatagcagcaacagcagtgacagcagtgatagcagcaacagcagtgatagcagtgaaagcagtaatagtagtgacaacagcaatagcagtgacagcagcaacagcagtgacagcagtgatagcagtgacagcagtaatagtagtgacagcagcaatagcggtgacagcagcaacagcagtgacagcagtgatagcaatagcagcgacagcagtgacagcagcaacagcagcgatagcagtgacagcagtgatagcagtgacagcagtgacagcagtgatagcagcaacagcagtgatagcagtgacagcagtgacagcagtgatagcagtaatagtagtgacagcagcaacagcagtgacagcagcgatagcagtgacagcagcgatagcagtgacagcagtgacagcagcaatagcagtgacagcagtgacagcagcgacagcagtgatagcagtgacagcagtggcagcagcgacagcagtgatagcagtgacagcagtgatagcagcgatagcagtgacagcagcgacagcagtgacagcagtgacagcagtgaaagcagcgacagcagcgatagcagcgacagcagtgacagcagcgacagcagtgacagcagcgatagcagcgacagcagcgacagcagcgatagcagtgacagcagcaatagcagtgatagcagcgacagcagtgatagcagtgacagcagcgacagcagcgatagcagcgacagcagtgatagtagtgatagcagtgacagcagtgacagcagcgacagcagtgacagcagcgacagcagtgacagcagcgacagcagtgacagcaatgaaagcagcgacagcagtgacagcagcgatagcagtgacagcagcaacagcagtgacagcagcgacagcagtgatagcagtgacagcacatctgacagcaatgatgagagtgacagccagagcaagtctggtaacggtaacaacaatggaagtgacagtgacagtgacagtgaaggcagtgacagtaaccactcaaccagtgatgattagaacaaaagaaaaacccataagattccttttgtgaaaagtttggtaatgggataggaaaaaaagatttccaagaaagtaaagaaaggggagaaataaacataagacgtatgtaaacaaaaacaactgggggaatcaaatcaaacagttggattcagaaccaagacctaactcctgcagagacagactctgaatgcatgacctttggtacatgcctgttaatattcatgttctgaaaatattttgttaaaagtgtaaatctaaacataaaagaacaattaaaatattctttaatacttcacacagaaacaattaaaatattctttaatacttcacacagaa

SEQ ID NO: 9 - protein, 396 aa, BMP
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 10 - DNA, 1581 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatagtacagcaaaattaaatacataaatatatatata

SEQ ID NO: 11 - DNA, 42 bp, Unknown
  Synthetic
ggatggagctgtatcatcctcttcttggtagcaacagctaca

SEQ ID NO: 12 - DNA, 34 bp, Unknown
  Synthetic
ctaatgtcgacatggagagtggcagccgtggaga

SEQ ID NO: 13 - DNA, 34 bp, Unknown
  Synthetic
gcattctagattaaagcacccgccattcaaatcg